SEQUENCE LISTING (16 sequences)

SEQ ID NO: 1 - protein, 773 aa, unidentified
MILDTDYITEDGKPVIRIFKKENGEFKIDYDRNFEPYIYALLKDDSAIEDVKKITAERHGTTVRVVRAEKVKKKFLGRPIEVWKLYFTHPQDVPAIRDKIKEHPAVVDIYEYDIPFAKRYLIDKGLIPMEGDEELKMLAFDIETLYHEGEEFAEGPILMISYADEEGARVITWKNIDLPYVDVVSTEKEMIKRFLKVVKEKDPDVLITYNGDNFDFAYLKKRSEKLGVKFILGREGSEPKIQRMGDRFAVEVKGRIHFDLYPVIRRTINLPTYTLEAVYEAIFGQPKEKVYAEEIAQAWETGEGLERVARYSMEDAKVTYELGKEFFPMEAQLSRLVGQSLWDVSRSSTGNLVEWFLLRKAYERNELAPNKPDERELARRRESYAGGYVKEPERGLWENIVYLDFRSLYPSIIITHNVSPDTLNREGCEEYDVAPQVGHKFCKDFPGFIPSLLGDLLEERQKVKKKMKATIDPIEKKLLDYRQRAIKILANSFYGYYGYAKARWYCKECAESVTAWGRQYIETTIREIEEKFGFKVLYADTDGFFATIPGADAETVKKKAKEFLDYINAKLPGLLELEYEGFYKRGFFVTKKKYAVIDEEDKITTRGLEIVRRDWSEIAKETQARVLEAILKHGDVEEAVRIVKEVTEKLSKYEVPPEKLVIYEQITRDLKDYKATGPHVAVAKRLAARGIKIRPGTVISYIVLKGSGRIGDRAIPFDEFDPAKHKYDAEYYIENQVLPAVERILRAFGYRKEDLRYQKTRQVGLGAWLKPKT

SEQ ID NO: 2 - protein, 773 aa, synthetic construct
MVLDTDYITEDGKPVIRIFKKENGEFKIDYDRNFEPYLYALLKDDSAIEDVKKITAERHGTTVRVVRAEKVKKKFLGRPIEVWKLYFTHPQDVPAIMDKIKEHPAVVDIYEYDIPFAIRYLIDKGLIPMEGDEELKLLAFDIETLYHEGEEFAEGPILMISYADEEGARVITWKNIDLPYVDVVSTEKEMIKRFLKVVKEKDPDVLITYNGDNFDFAYLKKRSEKLGVKFILGREGSEPKIQRMGDRFAVEVKGRIHFDLYPVIRRTINLPTYTLEAVYEAIFGQPKEKVYAEEIAQAWETGEGLERVARYSMEDAKVTYELGKEFFPMEAQLSRLVGQSLWDVSRSSTGNLVEWFLLRKAYERNELAPNKPDERELARRHESHAGGYIKEPERGLWENIVYLDFRSLYPSIIITHNVSPDTLNREGCEEYDVAPQVGHKFCKDFPGFIPSLLGDLLEERQKVKKRMKATIDPIEKKLLDYRQRAIKILANSLYGYYGYAKARWYCKECAESVIAWGRQYLETTIREIEEKFGFKVLYADTDGFFATIPGADAETVKKKAKEFLDYINAKLPGLLELEYEGFYKRGLFVTKKKYAVIDEEDKITTRGLEIVRRDWSEIAKETQARVLEAILKHGDVEEAVRIVKEVTEKLSKYEVPPEKLVIYKQITRDLKDYKATGPHVAVAKRLAARGIKIRPGTVISYIVLKGSGRIVDRAIPFDEFDPAKHKYDAEYYIEKQVLPAVERILRAFGYRKEDLRYQKTRQVGLGARLKPKT

SEQ ID NO: 3 - protein, 773 aa, synthetic construct
MVLDTDYITEDGKPVIRIFKKENGEFKIDYDRNFEPYLYALLKDDSAIEDVKKITAERHGTTVRVVRAEKVKKKFLGRPIEVWKLYFTHPQDVPAIMDKIKEHPAVVDIYEYDIPFAIRYLIDKGLIPMEGDEELKLLAFAIATLYHEGEEFAEGPILMISYADEEGARVITWKNIDLPYVDVVSTEKEMIKRFLKVVKEKDPDVLITYNGDNFDFAYLKKRSEKLGVKFILGREGSEPKIQRMGDRFAVEVKGRIHFDLYPVIRRTINLPTYTLEAVYEAIFGQPKEKVYAEEIAQAWETGEGLERVARYSMEDAKVTYELGKEFFPMEAQLSRLVGQSLWDVSRSSTGNLVEWFLLRKAYERNELAPNKPDERELARRHESHAGGYIKEPERGLWENIVYLDFRSLYPSIIITHNVSPDTLNREGCEEYDVAPQVGHKFCKDFPGFIPSLLGDLLEERQKVKKRMKATIDPIEKKLLDYRQRAIKILANSLYGYYGYAKARWYCKECAESVIAWGRQYLETTIREIEEKFGFKVLYADTDGFFATIPGADAETVKKKAKEFLDYINAKLPGLLELEYEGFYKRGLFVTKKKYAVIDEEDKITTRGLEIVRRDWSEIAKETQARVLEAILKHGDVEEAVRIVKEVTEKLSKYEVPPEKLVIYKQITRDLKDYKATGPHVAVAKRLAARGIKIRPGTVISYIVLKGSGRIVDRAIPFDEFDPAKHKYDAEYYIEKQVLPAVERILRAFGYRKEDLRYQKTRQVGLGARLKPKT

SEQ ID NO: 4 - protein, 873 aa, synthetic construct
MVLDTDYITEDGKPVIRIFKKENGEFKIDYDRNFEPYLYALLKDDSAIEDVKKITAERHGTTVRVVRAEKVKKKFLGRPIEVWKLYFTHPQDVPAIMDKIKEHPAVVDIYEYDIPFAIRYLIDKGLIPMEGDEELKLLAFAIATLYHEGEEFAEGPILMISYADEEGARVITWKNIDLPYVDVVSTEKEMIKRFLKVVKEKDPDVLITYNGDNFDFAYLKKRSEKLGVKFILGREGSEPKIQRMGDRFAVEVKGRIHFDLYPVIRRTINLPTYTLEAVYEAIFGQPKEKVYAEEIAQAWETGEGLERVARYSMEDAKVTYELGKEFFPMEAQLSRLVGQSLWDVSRSSTGNLVEWFLLRKAYERNELAPNKPDERELARRHESHAGGYIKEPERGLWENIVYLDFRSLYPSIIITHNVSPDTLNREGCEEYDVAPQVGHKFCKDFPGFIPSLLGDLLEERQKVKKRMKATIDPIEKKLLDYRQRAIKILANSLYGYYGYAKARWYCKECAESVIAWGRQYLETTIREIEEKFGFKVLYADTDGFFATIPGADAETVKKKAKEFLDYINAKLPGLLELEYEGFYKRGLFVTKKKYAVIDEEDKITTRGLEIVRRDWSEIAKETQARVLEAILKHGDVEEAVRIVKEVTEKLSKYEVPPEKLVIYKQITRDLKDYKATGPHVAVAKRLAARGIKIRPGTVISYIVLKGSGRIVDRAIPFDEFDPAKHKYDAEYYIEKQVLPAVERILRAFGYRKEDLRYQKTRQVGLGARLKPKTGGSMAAKKTVTKADLVDQVAQATGLKKKDVKAMVDALLAKVEEALANGSKVQLTGFGTFEVRKRKARTGVKPGTKEKIKIPATQYPAFKPGKALKDKVKK

SEQ ID NO: 5 - protein, 872 aa, synthetic construct
MVLDTDYITEDGKPVIRIFKKENGEFKIDYDRNFEPYLYALLKDDSAIEDVKKITAERHGTTVRVVRAEKVKKKFLGRPIEVWKLYFTHPQDVPAIMDKIKEHPAVVDIYEYDIPFAIRYLIDKGLIPMEGDEELKLLAFDIETLYHEGEEFAEGPILMISYADEEGARVITWKNIDLPYVDVVSTEKEMIKRFLKVVKEKDPDVLITYNGDNFDFAYLKKRSEKLGVKFILGREGSEPKIQRMGDRFAVEVKGRIHFDLYPVIRRTINLPTYTLEAVYEAIFGQPKEKVYAEEIAQAWETGEGLERVARYSMEDAKVTYELGKEFFPMEAQLSRLVGQSLWDVSRSSTGNLVEWFLLRKAYERNELAPNKPDERELARRHESHAGGYIKEPERGLWENIVYLDFRSLYPSIIITHNVSPDTLNREGCEEYDVAPQVGHKFCKDFPGFIPSLLGDLLEERQKVKKRMKATIDPIEKKLLDYRQRAIKILANSLYGYYGYAKARWYCKECAESVIAWGRQYLETTIREIEEKFGFKVLYADTDGFFATIPGADAETVKKKAKEFLDYINAKLPGLLELEYEGFYKRGLFVTKKKYAVIDEEDKITTRGLEIVRRDWSEIAKETQARVLEAILKHGDVEEAVRIVKEVTEKLSKYEVPPEKLVIYKQITRDLKDYKATGPHVAVAKRLAARGIKIRPGTVISYIVLKGSGRIVDRAIPFDEFDPAKHKYDAEYYIEKQVLPAVERILRAFGYRKEDLRYQKTRQVGLGARLKPKGGSMAAKKTVTKADLVDQVAQATGLKKKDVKAMVDALLAKVEEALANGSKVQLTGFGTFEVRKRKARTGVKPGTKEKIKIPATQYPAFKPGKALKDKVKK

SEQ ID NO: 6 - protein, 97 aa, Thermus sp.
MAAKKTVTKADLVDQVAQATGLKKKDVKAMVDALLAKVEEALANGSKVQLTGFGTFEVRKRKARTGVKPGTKEKIKIPATQYPAFKPGKALKDKVKK

SEQ ID NO: 7 - protein, 774 aa, unidentified
MILDTDYITEDGKPVIRIFKKENGEFKIEYDRTFEPYFYALLKDDSAIEEVKKITAERHGTVVTVKRVEKVQKKFLGRPVEVWKLYFTHPQDVPAIRDKIREHPAVIDIYEYDIPFAKRYLIDKGLVPMEGDEELKMLAFDIETLYEEGEEFAEGPILMISYADEEGARVITWKNVDLPYVDVVSTEREMIKRFLRVVKEKDPDVLITYNGDNFDFAYLKKRCEKLGINFALGRDGSEPKIQRMGDRFAVEVKGRIHFDLYPVIRRTINLPTYTLEAVYEAVFGQPKEKVYAEEITTAWETGENLERVARYSMEDAKVTYELGKEFLPMEAQLSRLIGQSLWDVSRSSTGNLVEWFLLRKAYERNELAPNKPDEKELARRRQSYEGGYVKEPERGLWENIVYLDFRSLYPSIIITHNVSPDTLNREGCKEYDVAPQVGHRFCKDFPGFIPSLLGDLLEERQKIKKKMKATIDPIERKLLDYRQRAIKILANSYYGYYGYARARWYCKECAESVTAWGREYITMTIKEIEEKYGFKVIYSDTDGFFATIPGADAETVKKKAMEFLKYINAKLPGALELEYEGFYERGFFVTKKKYAVIDEEGKITTRGLEIVRRDWSEIAKETQARVLEALLKDGDVEKAVRIVKEVTEKLSKYEVPPEKLVIHEQITRDLKDYKATGPHVAVAKRLAARGVKIRPGTVISYIVLKGSGRIGDRAIPFDEFDPTKHKYDAEYYIENQVLPAVERILRAFGYRKEDLRYQKTRQVGLSAWLKPKGT

SEQ ID NO: 8 - protein, 775 aa, synthetic construct
MILDTDYITENGKPVIRVFKKENGEFKIEYDRTFEPYFYALLKDDSAIEDVKKVTAKRHGTVVKVKRAEKVQKKFLGRPIEVWKLYFNHPQDVPAIRDRIRAHPAVVDIYEYDIPFAKRYLIDKGLIPMEGDEELTMLAFAIATLYHEGEEFGTGPILMISYADGSEARVITWKKIDLPYVDVVSTEKEMIKRFLRVVREKDPDVLITYNGDNFDFAYLKKRCEELGIKFTLGRDGSEPKIQRMGDRFAVEVKGRIHFDLYPVIRRTINLPTYTLEAVYEAVFGKPKEKVYAEEIAQAWESGEGLERVARYSMEDAKVTYELGREFFPMEAQLSRLIGQSLWDVSRSSTGNLVEWFLLRKAYKRNELAPNKPDERELARRRGGYAGGYVKEPERGLWDNIVYLDFRSLYPSIIITHNVSPDTLNREGCKEYDVAPEVGHKFCKDFPGFIPSLLGDLLEERQKIKRKMKATVDPLEKKLLDYRQRLIKILANSFYGYYGYAKARWYCKECAESVTAWGREYIEMVIRELEEKFGFKVLYADTDGLHATIPGADAETVKKKAKEFLKYINPKLPGLLELEYEGFYVRGFFVTKKKYAVIDEEGKITTRGLEIVRRDWSEIAKETQARVLEAILKHGDVEEAVRIVKEVTEKLSKYEVPPEKLVIHEQITRDLRDYKATGPHVAVAKRLAARGVKIRPGTVISYIVLKGSGRIGDRAIPADEFDPTKHRYDAEYYIENQVLPAVERILKAFGYRKEDLRYQKTKQVGLGAWLKVKGKK

SEQ ID NO: 9 - protein, 6 aa, synthetic construct
HHHHHH

SEQ ID NO: 10 - protein, 11 aa, synthetic construct
SEEDEEKEEDG

SEQ ID NO: 11 - protein, 7 aa, synthetic construct
ENLYFQG

SEQ ID NO: 12 - protein, 5 aa, synthetic construct
DDDDK

SEQ ID NO: 13 - protein, 4 aa, synthetic construct
IEGR

SEQ ID NO: 14 - protein, 6 aa, synthetic construct
LVPRGS

SEQ ID NO: 15 - protein, 8 aa, synthetic construct
LEVLFQGP

SEQ ID NO: 16 - protein, 251 aa, synthetic construct
MSGPFEVVFDGAKEFADLIATASNLIDEAAFKFTEEGISMRAMDPSRVVLIDLNLPESIFSKYEVEEPETIGINMDQFKKILKRGKAKDTLILRKGDENFLEITFEGTAKRTFRLPLIDVEELELELPELPFTAKVVLLGEVLKEGIKDASLVSDAIKFIAKENEFTMKAEGETNEVEIRLTLEDEGLLDLEVEEETKSAYGIRYLSDMVKGIGKADEVILRFGNEMPLQMEYMIRDEGRLTFLLAPRVEE

(19) United States
(12) Patent Application Publication
(10) Pub. No.: US 2023/0374475 A1
(43) Pub. Date: Nov. 23, 2023

(54) ENGINEERED THERMOPHILIC REVERSE TRANSCRIPTASE

(71) Applicant: 10x Genomics, Inc., Pleasanton, CA (US)

(72) Inventor: Shankar SHASTRY, Pleasanton, CA (US)

(73) Assignee: 10x Genomics, Inc., Pleasanton, CA (US)

(21) Appl. No.: 18/319,864

(22) Filed: May 18, 2023

Related U.S. Application Data
(60) Provisional application No. 63/343,451, filed on May 18, 2022.

(51) Int. Cl. C12N 9/12; C12N 15/63; C12N 15/10

(52) U.S. Cl. CPC C12N 9/1241; C12Y 207/07049; C12N 15/63; C12N 15/102

(57) ABSTRACT

The present disclosure relates generally to engineered nucleic acid processing enzymes and derivatives thereof, compositions and kits comprising the same; and methods of generating and using the same.

### CROSS-REFERENCE TO RELATED APPLICATIONS

This application claims the benefit of priority to U.S. Provisional Patent Application No. 63/343,451, filed May 18, 2022, which is hereby incorporated by reference in its entirety for any and all purposes.

### SEQUENCE LISTING

The instant application contains a Sequence Listing which has been submitted electronically in ASCII format and is hereby incorporated by reference in its entirety. Said ASCII copy, created on May 18, 2023 is named 131488_0180_Sequence_Listing.xml and is 20,603 bytes.

### TECHNICAL FIELD

The present disclosure relates to the fields of molecular biology, cell biology, biochemistry, and diagnostics, as they pertain to genetic engineering of reverse transcriptase (RT) enzymes for the reverse transcription of nucleic acid molecules.

### BACKGROUND

The following description of the background of the present technology is provided simply as an aid in understanding the present technology and is not admitted to describe or constitute prior art to the present technology.

The discovery of reverse transcriptase (RT) in the 1970's revolutionized the understanding of eukaryotic biology by demonstrating that genetic information did not flow unidirectionally from DNA to RNA to proteins. Rather, the genetic information could also flow in the reverse direction from RNA back to DNA. The ability to convert mature mRNA back into cDNA, without the introns present in genomic DNA is critical for obtaining information in a wide variety of biomedical contexts, including diagnostics, prognostics, biotechnology, and forensic biology. Since then, RT enzymes (RTs) have become ubiquitous tools in molecular biology driving enabling technologies such as next-generation RNA-Sequencing, Maxam-Gilbert sequencing and chain-termination methods, or de novo sequencing methods including shotgun sequencing and bridge PCR, or next-generation methods including polony sequencing, 454 pyrosequencing, Illumina sequencing, SOLiD sequencing, Ion Torrent semiconductor sequencing, HeliScope single molecule sequencing, SMRT® sequencing.

RT enzymes were initially found in retroviruses such as Moloney murine leukemia virus (MMLV)). It is now clear that RTs are present in other microorganisms, including transposable elements, where RTs are responsible for converting an RNA genome of these organisms into DNA to facilitate the integration of the microorganisms into a host's chromosome. All known natural RTs are derived from a shared common ancestor. Generally, RTs are mesophilic enzymes that function best at moderate temperatures ranging from 20° C. to 45° C. The mesophilic nature of RTs is problematic for in vitro amplification reactions because RNAs tend to adopt stable secondary structures at lower temperatures resulting in inefficient reverse transcription reactions at these low to moderate temperatures. In addition to the RNA secondary structures, RT reactions and amplification reactions also fail because biological samples from which nucleic acids are extracted often contain additional compounds that are inhibitory to reverse transcription and/or amplification reactions. This inhibition is particularly problematic when the volume of an amplification reaction is very small (e.g., nanoliter), such as in single cell profiling reactions and additional methods where small reaction volumes are preferential.

Accordingly, there is a need for improved reverse transcriptases with improved properties, such as improved efficiency, processivity, thermoreactivity, and/or thermostability. The present disclosure addresses this need.

### SUMMARY OF THE PRESENT TECHNOLOGY

The present disclosure provides engineered recombinant archeal Family-B polymerases that have the fidelity and thermostability of known DNA polymerases in combination with a reverse transcriptase activity. In certain aspects the invention provides engineered enzymes that produce cDNA from an RNA template at high temperatures, for example without limitation temperatures >50° C., >55° C., >60° C., >65° C., or higher. Thus, in certain embodiments, engineered enzymes of the disclosure have the following characteristics: thermostability, the ability to reverse transcribe RNA templates, including long RNA templates; and optionally proofreading. In certain embodiments, the engineered enzymes of invention permit melting of RNA structure(s) and generating cDNA copies. In certain embodiments, the engineered enzymes of the invention have high thermostability, e.g., thermostability at temperatures above 50° C., 51° C., 52° C., 53° C., 54° C., 55° C., 56° C., 57° C., 58° C., 59° C., 60° C., 61° C., 62° C., 63° C., 64° C., 65° C., 66° C., 67° C., 68° C., 69° C., 70° C., 71° C., 72° C., 73° C. or more, and optionally have proofreading activity. In certain embodiments, the engineered enzymes of the invention have high thermostability, e.g., thermostability at temperatures of 50° C., 51° C., 52° C., 53° C., 54° C., 55° C., 56° C., 57° C., 58° C., 59° C., 60° C., 61° C., 62° C., 63° C., 64° C., 65° C., 66° C., 67° C., 68° C., 69° C., 70° C., 71° C., 72° C., 73° C. or more, and optionally have proofreading activity. In certain embodiments, the engineered enzymes of the invention have high thermostability, e.g., thermostability at temperatures of about: 50° C., 51° C., 52° C., 53° C., 54° C., 55° C., 56° C., 57° C., 58° C., 59° C., 60° C., 61° C., 62° C., 63° C., 64° C., 65° C., 66° C., 67° C., 68° C., 69° C., 70° C., 71° C., 72° C., 73° C. or more, and optionally have proofreading activity. In specific embodiments, the engineered enzymes of the present disclosure are more processive and/or more primer-dependent, resulting in less promiscuity in generating an accurate cDNA imprint of a mRNA. In certain embodiments, because of their proofreading domain, the enzymes of the present disclosure generate fewer mutations than other enzymes and provide a more accurate representation of the RNAs present in a given population, including without limitation, a sample from one or more individuals, from one or more cell types or population, single cells, or combination thereof.

In one aspect, the present disclosure provides an engineered nucleic acid processing enzyme comprising: (a) a first domain comprising a polymerase domain, wherein the polymerase domain comprises an amino acid sequence of an engineered Thermococcus gorgonarius polymerase (Tgo polymerase); and (b) a second domain conjugated to the first domain, wherein the second domain comprises a nucleic acid binding domain.

In some embodiments of the engineered nucleic acid processing enzyme described herein, the polymerase domain comprises an amino acid sequence having: (a) at least 90%, at least 91%, at least 92%, at least 93%, at least 94%, at least 95%, at least 96%, at least 97%, at least 98%, or at least 99% sequence identity to the amino acid sequence of SEQ ID NO: 1; (b) at least 95% identity to the amino acid sequence of SEQ ID NO: 1; (c) at least 97% identity to the amino acid sequence of SEQ ID NO: 1; (d) at least about 10, at least about 15, or at least about 20 substitutions in the amino acid sequence of SEQ ID NO: 1; (e) at least 97% identity to the amino acid sequence of SEQ ID NO: 1 and at least about 15 substitutions in the amino acid sequence of SEQ ID NO: 1; (f) 90%, 91%, 92%, 93%, 94%, 95%, 96%, 97%, 98%, or 99% sequence identity to the amino acid sequence of SEQ ID NO: 1.

In some embodiments, the polymerase domain comprises: (a) an aspartic acid substitution at position 141; (b) a glutamic acid substitution at position 143; (c) an alanine substitution at position 485; (d) a valine substitution at position 93; (e) an arginine substitution at position 97; (f) a tyrosine substitution at position 384; (g) a valine substitution at position 389; (h) a phenylalanine substitution at position 493; (i) a phenylalanine substitution at position 587; (j) a glutamic acid substitution at position 664; (k) a glycine substitution at position; (l) a tryptophan substitution at position 768; (m) an isoleucine substitution at position 2; (n) an isoleucine substitution at position 38; (o) a lysine substitution at position 118; (p) a methionine substitution at position 137; (q) an arginine substitution at position 381; (r) a lysine substitution at position 466; (s) a tyrosine substitution at position 514; (t) an isoleucine substitution at position 521; and/or (u) an asparagine substitution at position 735 in SEQ ID NO: 1.

In some embodiments, (a) the polymerase domain comprises a substitution at positions 141 and 143 of SEQ ID NO: 1 or 2; (b) the polymerase domain comprises a substitution at position 141 of SEQ ID NOs: 2; (c) the engineered nucleic acid processing enzyme lacks proofreading activity; or (d) the engineered nucleic acid processing enzyme comprises the amino acid sequence of SEQ ID NO: 1, 2 or 3.

In some embodiments, the polymerase domain comprises a combination of: (a) R97M, D141A, E143A, Y384H, V389I, Y493L, F587L, E664K, G711V, and W768R substitutions in SEQ ID NO: 1; (b) I2V, I38L, R97M, K118I, M137L, E143A, R381H; Y384H, V389I, K466R, F493L, T514I, I521L, F587L, E664K, G711V, N735K, and W768R substitutions in SEQ ID NO: 1; (c) I2V, I38L, R97M, K118I, M137L, D141A, E143A, R381H, Y384H, V389I, K466R, F493L, T514I, I521L, F587L, E664K, G711V, N735K, and W768R substitutions in SEQ ID NO: 1; or (d) I2V, I38L, V93Q, R97M, K118I, M137L, D141A, E143A, R381H, Y384H, A485L, V389I, K466R, F493L, T514I, I521L, F587L, E664K, G711V, N735K, and W768R substitutions in SEQ ID NO: 1.

In some embodiments of the engineered nucleic acid processing enzyme described herein, the nucleic acid binding domain comprises: (a) a nucleic acid binding protein selected from the group consisting of a histone-like protein, an archaeal basic nucleic acid binding protein, a basic DNA binding domain, HMf-like protein, HU-like protein, HU-family DNA binding protein, Sm-like protein domain, proliferating cell nuclear antigen (PCNA), HU, sto7, Sso7d, Sac7d, and Sac7e; (b) a T. kodakarensis PCNA; (c) a polynucleotide encoding the amino acid sequence of SEQ ID NO: 16 or a sequence having at least 90% sequence identity to the amino acid sequence of SEQ ID NO: 16; or (d) a Thermus thermophile HU-family DNA binding protein.

In some embodiments, the nucleic acid binding protein comprises an amino acid sequence having at least 90%, at least 91%, at least 92%, at least 93%, at least 94%, at least 95%, at least 96%, at least 97%, at least 98%, or at least 99% sequence identity to the amino acid sequence of SEQ ID NO: 10. In some embodiments, the nucleic acid binding protein comprises an amino acid sequence having 90%, 91%, 92%, 93%, 94%, 95%, 96%, 97%, 98%, or 99% sequence identity to the amino acid sequence of SEQ ID NO: 10.

In some embodiments, the nucleic acid binding domain comprises an amino acid sequence set forth in SEQ ID NO: 4, 5, or 6 or an amino acid sequence having at least 90% sequence identity to the amino acid sequence set forth in SEQ ID NO: 4, 5, or 6.

In some embodiments of the engineered nucleic acid processing enzyme described herein, the engineered nucleic acid processing enzyme further comprises a tag protein selected from the group consisting of an affinity tag, a fluorescent tag, or an expression and/or solubility enhancement tag.

In some embodiments, the engineered nucleic acid processing enzyme comprises: (a) an hexahistidine tag (his-tag); (b) an amino acid sequence of SEQ ID NO: 9; or an amino acid sequence having at least 90% sequence identity to the amino acid sequence of SEQ ID NO: 9; (c) a short peptide C-terminal tag; (d) an amino acid sequence of SEQ ID NO: 10; (e) an amino acid sequence having at least 90% sequence identity to the amino acid sequence of SEQ ID NO: 10; or (f) an endoprotein cleavage sequence comprising the amino acid sequence of SEQ ID NO: 11, SEQ ID NO: 12, SEQ ID NO: 13, SEQ ID NO: 14, or SEQ ID NO: 15.

In some embodiments of the engineered nucleic acid processing enzyme described herein, the engineered nucleic acid processing enzyme: (a) is thermophilic; and/or (b) is resistant to thermal inactivation when compared to a wild-type polymerase; or (c) is resistant to thermal inactivation at a temperature from about 53° C. to about 75° C.; from about 55° C. to about 75° C.; from about 60° C. to about 75° C.; from about 53° C. to about 68° C.; from about 55° C. to about 68° C.; from about 45° C. to about 68° C.; or from about 50° C. to about 68° C.; or (d) is resistant to thermal inactivation at a temperature of about 68° C.

In some embodiments, the engineered nucleic acid processing enzyme possesses enhanced half-life when compared to a wild-type polymerase at a temperature from about 53° C. to about 75° C.; from about 55° C. to about 75° C.; from about 60° C. to about 75° C.; from about 53° C. to about 68° C.; from about 55° C. to about 68° C.; from about 45° C. to about 68° C.; or from about 50° C. to about 68° C.

In another aspect, the present disclosure provides an isolated nucleic acid molecule encoding the engineered thermostable reverse transcriptase described herein.

In another aspect, the present disclosure provides an expression vector comprising the isolated nucleic acid molecule encoding the engineered thermostable reverse transcriptase described herein.

In another aspect, the present disclosure provides a host cell transfected with the expression vector or nucleic acid molecule described herein.

In another aspect, the present disclosure provides a method of using the engineered thermostable reverse transcriptase described herein, the method comprising contacting the engineered thermostable reverse transcriptase with a nucleic acid template under suitable conditions to produce a polymerized nucleic acid product, wherein the nucleic acid template is an RNA, a DNA, or a nucleic acid comprising an unnatural nucleotide.

In another aspect, the present disclosure provides a nucleic acid extension method comprising: (a) contacting a target nucleic acid molecule with the engineered nucleic acid processing enzyme of claim 1 and a plurality of nucleic acid barcoded molecules comprising a barcode sequence, and (b) incubating the target nucleic acid, the engineered nucleic acid processing enzyme and barcoded molecules under conditions in which the barcoded molecules are extended by the engineered thermostable reverse transcriptase. In some embodiments, (i) one of the plurality of nucleic acid barcoded molecules hybridizes to the target nucleic acid molecule; (ii) the nucleic acid binding domain binds and stabilizes the target nucleic acid molecule-barcoded molecule complex; and (iii) the polymerase domain extends the one of the plurality of nucleic acid barcoded molecules that is hybridized to the target nucleic acid molecule.

In some embodiments of the nucleic acid extension method described herein, the polymerase domain comprises an amino acid sequence having: (a) at least 90%, at least 91%, at least 92%, at least 93%, at least 94%, at least 95%, at least 96%, at least 97%, at least 98%, or at least 99% sequence identity to the amino acid sequence of SEQ ID NO: 1; (b) at least 95% identity to the amino acid sequence of SEQ ID NO: 1; (c) at least 97% identity to the amino acid sequence of SEQ ID NO: 1; (d) at least about 10, at least about 15, or at least about 20 substitutions in the amino acid sequence of SEQ ID NO: 1; or (e) at least 97% identity to the amino acid sequence of SEQ ID NO: 1 and at least about 15 substitutions in the amino acid sequence of SEQ ID NO: 1. In some embodiments of the nucleic acid extension method described herein, the polymerase domain comprises an amino acid sequence having 90%, 91%, 92%, 93%, 94%, 95%, 96%, 97%, 98%, or 99% sequence identity to the amino acid sequence of SEQ ID NO: 1.

In some embodiments: (a) the polymerase domain comprises a substitution at positions 141 and 143 of SEQ ID NO: 1 or 2; (b) the polymerase domain comprises a substitution at position 141 of SEQ ID NOs: 2; (c) the engineered nucleic acid processing enzyme lacks proofreading activity; or (d) the engineered nucleic acid processing enzyme comprises the amino acid sequence of SEQ ID NO: 1, 2 or 3; or (e) the nucleic acid binding protein comprises an amino acid sequence having at least 90%, at least 91%, at least 92%, at least 93%, at least 94%, at least 95%, at least 96%, at least 97%, at least 98%, or at least 99% sequence identity to the amino acid sequence of SEQ ID NO: 4, 5, 6, or 10; or (f) the nucleic acid binding domain comprises an amino acid sequence set forth in SEQ ID NO: 4, 5, or 6. In some embodiments, the nucleic acid binding protein comprises an amino acid sequence having 90%, 91%, 92%, 93%, 94%, 95%, 96%, 97%, 98%, or 99% sequence identity to the amino acid sequence of SEQ ID NO: 4, 5, 6, or 10.

In some embodiments, the target nucleic acid molecule is further contacted with a sliding clamp molecule selected from an archea, an eucarya, or a bacteriophage sliding clamp protein.

In some embodiments, the sliding clamp protein is selected from E. coli polymerase R subunit; T4 bacteriophage gp45; T. gorgonarius PCNA; or T. kodakarensis PCNA.

In one aspect, the present disclosure provides an engineered nucleic acid processing enzyme comprising: (a) a first domain comprising a polymerase domain; and (b) a second domain conjugated to the first domain. In some embodiments, the polymerase domain comprises an amino acid sequence of an engineered Thermococcus gorgonarius polymerase (Tgo polymerase). In some embodiments, the second domain comprises a nucleic acid binding domain.

In some embodiments, the polymerase comprises the amino acid sequence of SEQ ID NO: 1 or an amino acid sequence having at least 90% sequence identity to the amino acid sequence of SEQ ID NO: 1. In some embodiments, the polymerase comprises an amino acid sequence having: (a) at least 90%, at least 91%, at least 92%, at least 93%, at least 94%, at least 95%, at least 96%, at least 97%, at least 98%, or at least 99% sequence identity to the amino acid sequence of SEQ ID NO: 1; (b) at least 95% identity to the amino acid sequence of SEQ ID NO: 1; (c) at least 97% identity to the amino acid sequence of SEQ ID NO: 1; (d) at least about 10, at least about 15, or at least about 20 substitutions in the amino acid sequence of SEQ ID NO: 1; or (e) at least 97% identity to the amino acid sequence of SEQ ID NO: 1 and at least about 15 substitutions in the amino acid sequence of SEQ ID NO: 1. In some embodiments, the polymerase comprises an amino acid sequence having 90%, 91%, 92%, 93%, 94%, 95%, 96%, 97%, 98%, or 99% sequence identity to the amino acid sequence of SEQ ID NO: 1

In some embodiments of the engineered nucleic acid processing enzyme described herein, (a) the polymerase domain of the engineered nucleic acid processing enzyme comprises a substitution at positions 141 and 143 of SEQ ID NO: 1 or 2; (b) the polymerase domain comprises a substitution at position 141 of SEQ ID NOs: 2; (c) the engineered nucleic acid processing enzyme lacks proofreading activity; or (d) the engineered nucleic acid processing enzyme comprises the amino acid sequence of SEQ ID NO: 3.

In some embodiments of the engineered nucleic acid processing enzyme described herein, the polymerase domain comprises: (a) an aspartic acid substitution at position 141; (b) a glutamic acid substitution at position 143; (c) an alanine substitution at position 485; (d) a valine substitution at position 93; (e) an arginine substitution at position 97; (f) a tyrosine substitution at position 384; (g) a valine substitution at position 389; (h) a phenylalanine substitution at position 493; (i) a phenylalanine substitution at position 587; (j) a glutamic acid substitution at position 664; (k) a glycine substitution at position; (l) a tryptophan substitution at position 768; (m) an isoleucine substitution at position 2; (n) an isoleucine substitution at position 38; (o) a lysine substitution at position 118; (p) a methionine substitution at position 137; (q) an arginine substitution at position 381; (r) a lysine substitution at position 466; (s) a tyrosine substitution at position 514; (t) an isoleucine substitution at position 521; and/or (u) an asparagine substitution at position 735 in SEQ ID NO: 1.

In some embodiments of the engineered nucleic acid processing enzyme described herein, the polymerase domain comprises: (a) an aspartic acid to alanine substitution at position 141 (D141A); (b) a glutamic acid to alanine substitution at position 143 (E143A); (c) an alanine to leucine substitution at position 485 (A485L); (d) a valine to glutamine substitution at position 93 (V93Q); (e) an arginine to methionine substitution at position 97 (R97M); (f) a tyrosine to histidine substitution at position 384 (Y384H); (g) a valine to isoleucine substitution at position 389 (V389I); (h) a phenylalanine to leucine substitution at position 493 (F493L); (i) a phenylalanine to leucine substitution at position 587 (F587L); (j) a glutamic acid to lysine substitution at position 664 (E664K); (k) a glycine to valine substitution at position (G711V); (l) a tryptophan to arginine substitution at position 768 (W768R); (m) an isoleucine to valine substitution at position 2 (12V); (n) an isoleucine to leucine substitution at position 38 (I38L); (o) a lysine to isoleucine substitution at position 118 (K118I); (p) a methionine to leucine substitution at position 137 (M137L); (q) an arginine to histidine substitution at position 381 (R381H); (r) a lysine to arginine substitution at position 466 (K466R); (s) a tyrosine to isoleucine substitution at position 514 (T514I); (t) an isoleucine to leucine substitution at position 521 (I521L); and/or (u) an asparagine to lysine substitution at position 735 (N735K).

In some embodiments, the polymerase domain of the engineered nucleic acid processing enzyme described herein comprises a combination of: (a) R97M, D141A, E143A, Y384H, V389I, Y493L, F587L, E664K, G711V, and W768R substitutions in SEQ ID NO: 1; (b) I2V, I38L, R97M, K118I, M137L, E143A, R381H; Y384H, V389I, K466R, F493L, T514I, I521L, F587L, E664K, G711V, N735K, and W768R substitutions in SEQ ID NO: 1; (c) I2V, I38L, R97M, K118I, M137L, D141A, E143A, R381H, Y384H, V389I, K466R, F493L, T514I, I521L, F587L, E664K, G711V, N735K, and W768R substitutions in SEQ ID NO: 1; or (d) I2V, 138L, V93Q, R97M, K118I, M137L, D141A, E143A, R381H, Y384H, A485L, V389I, K466R, F493L, T514I, I521L, F587L, E664K, G711V, N735K, and W768R substitutions in SEQ ID NO: 1. In one embodiment, the polymerase domain comprises the amino acid sequence of SEQ ID NO: 2. In another embodiment, the polymerase domain comprises the amino acid sequence of SEQ ID NO: 3.

In some embodiments of the engineered nucleic acid processing enzyme described herein, the nucleic acid binding domain of the engineered nucleic acid processing enzyme described herein comprises: (a) a single stranded DNA binding protein; (b) a double stranded DNA binding protein; (c) a single stranded RNA binding protein; (d) a double stranded RNA binding protein; (e) a continuous RNA-DNA hybrid binding protein; or (d) a discontinuous RNA-DNA hybrid binding protein.

In some embodiments, the nucleic acid binding domain comprises a nucleic acid binding protein selected from the group consisting of a histone-like protein, an archaeal basic nucleic acid binding protein, a basic DNA binding domain, HMf-like protein, HU-like protein, HU-family DNA binding protein, Sm-like protein domain, proliferating cell nuclear antigen (PCNA), HU, sto7, Sso7d, Sac7d, and Sac7e.

In some embodiments of the engineered nucleic acid processing enzyme described herein, (a) the nucleic acid binding domain comprises a histone-like protein; (b) the nucleic acid binding domain is a PCNA, optionally a T. kodakarensis PCNA; or (c) the nucleic acid binding domain comprises a polynucleotide encoding the amino acid sequence of SEQ ID NO: 16 or a sequence having at least 90% sequence identity to the amino acid sequence of SEQ ID NO: 16.

In some embodiments, the nucleic acid binding domain comprises a bacterial histone-like protein or a bacterial HU-family DNA binding protein. In one embodiment, the nucleic acid binding domain is a Thermus thermophile HU-family DNA binding protein. In some embodiments, the nucleic acid binding domain binds a DNA-RNA hybrid complex, and the DNA-RNA hybrid is continuous or discontinuous.

In some embodiments, the nucleic acid binding protein comprises an amino acid sequence having at least 90%, at least 91%, at least 92%, at least 93%, at least 94%, at least 95%, at least 96%, at least 97%, at least 98%, or at least 99% sequence identity to the amino acid sequence of SEQ ID NO: 10. In some embodiments, the nucleic acid binding protein comprises an amino acid sequence having 90%, 91%, 92%, 93%, 94%, 95%, 96%, 97%, 98%, or 99% sequence identity to the amino acid sequence of SEQ ID NO: 10. In one embodiment, the nucleic acid binding domain comprises an amino acid sequence set forth in SEQ ID NO: 6 or an amino acid sequence having at least 90% sequence identity to the amino acid sequence set forth in SEQ ID NO: 6.

In some embodiments of the engineered nucleic acid processing enzyme described herein, the engineered nucleic acid processing enzyme comprises an amino acid sequence of SEQ ID NO: 5; or an amino acid sequence having at least 90% sequence identity to the amino acid sequence set forth in SEQ ID NO: 5. In some embodiments, the engineered nucleic acid processing enzyme comprises an amino acid sequence of SEQ ID NO: 4; or an amino acid sequence having at least 90% sequence identity to the amino acid sequence of SEQ ID NO: 4.

In some embodiments of the engineered nucleic acid processing enzyme described herein, the engineered nucleic acid processing enzyme further comprises a tag protein selected from the group consisting of an affinity tag, a fluorescent tag, or an expression and/or solubility enhancement tag. In some embodiments, the tag is selected from hexahistidine tag (his-tag), small ubiquitin-like modifier tag (SUMO), aVariFlex C-Terminal solubility enhancement tag, a short peptide C-terminal tag, Thioredoxin (Trx) tag, aVariFlex C-Terminal solubility enhancement tag, Solubility-enhancer peptide sequences (SET) tag, IgG domain B1 of Protein G (GB1) tag, IgG repeat domain ZZ of Protein A (ZZ) tag, Solubility enhancing Ubiquitous Tag (SNUT tag), Seventeen kilodalton protein (Skp tag), Phage T7 protein kinase (T7PK) tag, E. coli secreted protein A (EspA) tag, Monomeric bacteriophage T7 0.3 protein (Orc protein) (Mocr) tag, E. coli trypsin inhibitor (Ecotin) tag, Calcium-binding protein (CaBP) tag, Stress-responsive arsenate reductase (ArsC) tag, N-terminal fragment of translation initiation factor IF2 (IF2-domain I) tag, N-terminal fragment of translation initiation factor IF2 (Expressivity) tag, Fasciola hepatica 8-kDa antigen tag (Fh8), Glutathione-S-transferase (GST) tag, maltose-binding protein tag (MBP), FLAg tag peptide (FLAG), streptavidin binding peptide tag (Strep-II; strep), calmodulin-binding protein tag (CBP), mutated dehalogenase tag (HaloTag), staphylococcal Protein A (Protein A), intein mediated purification with the chitin-binding domain (IMPACT (CBD)), cellulose-binding module (CBM), dockerin domain of Clostridium josui tag (Dock), fungal avidin-like protein (Tamavidin).

In some embodiments, the tag is an affinity tag selected from hexahistidine tag (his-tag), Fasciola hepatica 8-kDa antigen tag (Fh8), Glutathione-S-transferase (GST) tag, maltose-binding protein tag (MBP), FLAg tag peptide (FLAG), streptavidin binding peptide tag (Strep-II), calmodulin-binding protein tag (CBP), mutated dehalogenase tag (HaloTag), staphylococcal Protein A (Protein A), intein mediated purification with the chitin-binding domain (IMPACT (CBD)), cellulose-binding module (CBM), dockerin domain of Clostridium josui tag (Dock), fungal avidin-like protein (Tamavidin). In some embodiments, the engineered nucleic acid processing enzyme comprises: (a) an hexahistidine tag (his-tag); or (b) an amino acid sequence of SEQ ID NO: 9; or an amino acid sequence having at least 90% sequence identity to the amino acid sequence of SEQ ID NO: 9.

In some embodiments, the engineered nucleic acid processing enzyme comprises a solubility enhancer tag selected from the group consisting of a SUMO tag, a GST tag, a Trx tag, aVariFlex C-Terminal solubility enhancement tag, a short peptide C-terminal tag, an Fh8 tag, MBP tag, SET tag, GB1 tag, ZZ tag, HaloTag, SNUT tag, Skp tag, T7PK tag, EspA tag, Mocr tag, Ecotin tag, CaBO tag, ArsC tag, IF2-domain I tag, Expressivity tag, RpoA, tag, SlyD, tag, Tsf tag, RpoS tag, PotD tag, Crr tag, msyB tag, yigD tag, and rpoD tag.

In some embodiments, the engineered nucleic acid processing enzyme comprises: (a) a short peptide C-terminal tag; (b) an amino acid sequence of SEQ ID NO: 10; or (c) an amino acid sequence having at least 90% sequence identity to the amino acid sequence of SEQ ID NO: 10.

In some embodiments, the tag further comprises: (a) an endoprotein cleavage sequence; (b) a cleavage sequence recognized by an endoprotein selected from the group consisting of alanine carboxypeptidase, Armillaria mellea astacin, bacterial leucyl aminopeptidase, cancer procoagulant, cathepsin B, clostripain, cytosol alanyl aminopeptidase, elastase, endoproteinase Arg-C, enterokinase (EnTK), gastricsin, gelatinase, Gly-X carboxypeptidase, glycyl endopeptidase, human rhinovirus 3C protease, hypodermin C, Iga-specific serine endopeptidase, leucyl aminopeptidase, leucyl endopeptidase, lysC, lysosomal pro-X carboxypeptidase, lysyl aminopeptidase, methionyl aminopeptidase, myxobacter, nardilysin, pancreatic endopeptidase E, picornain 2A, picornain 3C, proendopeptidase, prolyl aminopeptidase, proprotein convertase I, proprotein convertase II, russellysin, saccharopepsin, semenogelase, T-plasminogen activator, thrombin (Thr), tissue kallikrein, tobacco etch virus (TEV), togavirin, tryptophanyl aminopeptidase, U-plasminogen activator, V8, venombin A, venombin AB, factor Xa (Xa), and Xaa-pro aminopeptidase; or (c) an endoprotein cleavage sequence comprising the amino acid sequence of SEQ ID NO: 11, SEQ ID NO: 12, SEQ ID NO: 13, SEQ ID NO: 14, or SEQ ID NO: 15.

In some embodiments of the engineered nucleic acid processing enzyme described herein, the engineered nucleic acid processing enzyme: (a) is thermophilic; (b) is resistant to thermal inactivation when compared to a wild-type or control polymerase and/or RT; or (c) is resistant to thermal inactivation at a temperature from about 45° C. to about 50° C., from about 47° C. to about 52° C., from about 49° C. to about 53° C., from about 50° C. to about 55° C., from about 50° C. to about 60° C., from about 53° C. to about 75° C.; from about 55° C. to about 75° C.; from about 60° C. to about 75° C.; from about 53° C. to about 68° C.; from about 55° C. to about 68° C.; from about 45° C. to about 68° C.; or from about 50° C. to about 68° C.; and/or (d) is resistant to thermal inactivation at a temperature of about 68° C.

In some embodiments, the engineered nucleic acid processing enzyme possesses enhanced half-life when compared to a wild-type or control polymerase and/or RT at a temperature from about 53° C. to about 75° C.; from about 55° C. to about 75° C.; from about 60° C. to about 75° C.; from about 53° C. to about 68° C.; from about 55° C. to about 68° C.; from about 45° C. to about 68° C.; or from about 50° C. to about 68° C. In certain embodiments, increased half-life is associated with increased activity. In certain embodiments, the increased activity is measured at temperatures of 50° C., 51° C., 52° C., 53° C., 54° C., 55° C., 56° C., 57° C., 58° C., 59° C., 60° C., 61° C., 62° C., 63° C., 64° C., 65° C., 66° C., 67° C., 68° C., 69° C., 70° C., 71° C., 72° C., 73° C. or more. In certain embodiments, the increased activity is measured at temperatures of about: 50° C., 51° C., 52° C., 53° C., 54° C., 55° C., 56° C., 57° C., 58° C., 59° C., 60° C., 61° C., 62° C., 63° C., 64° C., 65° C., 66° C., 67° C., 68° C., 69° C., 70° C., 71° C., 72° C., 73° C. or more.

In some embodiments of the engineered nucleic acid processing enzyme described herein, the engineered nucleic acid processing enzyme possesses one or more of the following characteristics when compared to a wild-type polymerase: (a) increased thermostability; (b) increased thermoreactivity; (c) increased resistance to reverse transcriptase inhibitors; (d) increased ability to reverse transcribe difficult templates; (e) increased speed; (f) increased processivity; (g) increased specificity; (h) enhanced polymerization activity; or (i) increased sensitivity.

In some embodiments, (a) the increase in thermoreactivity, resistance to reverse transcriptase inhibitors, ability to reverse transcribe difficult templates, speed, processivity, specificity, or sensitivity is about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 90%, or about 100% as compared to the wild-type polymerase. In some embodiments, the polymerization activity is enhanced by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 90%, or about 100% as compared to the wild-type polymerase.

In some embodiments of the engineered nucleic acid processing enzyme described herein, the engineered nucleic acid processing enzyme has a dual DNA and RNA polymerase activity, or an RNA reverse transcriptase activity. In some embodiments, the thermostable chimeric polymerize enzyme reverse transcribes a RNA molecule having at least about 100, at least about 200, at least about 300, at least about 400, at least about 500, at least about 600, at least about 700, at least about 800, at least about 900, or at least about 1000 nucleotides.

In some embodiments, the thermostable chimeric polymerize enzyme reverse transcribes a RNA molecule that is at least about 1 kb, at least about 2 kb, at least about 3 kb, at least about 4 kb, at least about 5 kb, at least about 6 kb, at least about 7 kb, at least about 8 kb, at least about 9 kb, at least about 10 kb, at least about 11 kb, at least about 12 kb, at least about 13 kb, at least about 14 kb, or at least about 15 kb. In some embodiments, the thermostable chimeric polymerize enzyme reverse transcribes a RNA molecule that is at least about 7 kb or at least about 8 kb.

On aspect of the present disclosure provides an isolated nucleic acid molecule encoding the engineered thermostable reverse transcriptase described herein.

Another aspect of the present disclosure provides an expression vector comprising the isolated nucleic acid disclosed herein.

Another aspect of the present disclosure provides a host cell transfected with the expression vector disclosed herein.

One aspect of the present disclosure provides a method of using the engineered thermostable reverse transcriptase disclosed herein, the method comprising contacting the engineered thermostable reverse transcriptase with a nucleic acid template under suitable conditions to produce a polymerized nucleic acid product. In some embodiments, the nucleic acid template is an RNA, a DNA, or a nucleic acid comprising an unnatural nucleotide.

One aspect of the present disclosure provides a nucleic acid extension method comprising: (a) contacting a target nucleic acid molecule with an engineered nucleic acid processing enzyme and a plurality of nucleic acid barcoded molecules comprising a barcode sequence, and (b) incubating the target nucleic acid, the engineered nucleic acid processing enzyme and barcoded molecules under conditions in which the barcoded molecules are extended by the engineered thermostable reverse transcriptase. In some embodiment, (i) the engineered nucleic acid processing enzyme comprises: (1) a first domain comprising a polymerase domain; and (2) a second domain conjugated to the first domain; and (ii) one of the plurality of nucleic acid barcoded molecules hybridizes to the target nucleic acid molecule; (iii) the nucleic acid binding domain binds and stabilizes the target nucleic acid molecule-barcoded molecule complex; and (iv) the polymerase domain extends the one of the plurality of nucleic acid barcoded molecules that is hybridized to the target nucleic acid molecule. In some embodiments, the polymerase domain comprises an amino acid sequence of an engineered Thermococcus gorgonarius polymerase (Tgo polymerase). In some embodiments, the second domain comprises a nucleic acid binding domain.

In some embodiments of the nucleic acid extension method disclosed herein, (a) the nucleic acid is a ribonucleic acid (RNA) molecule; and (b) the engineered nucleic acid processing enzyme reverse transcribes the RNA molecule into a complementary DNA, and then amplifies the complementary DNA into a nucleic acid product in the same reaction.

In some embodiments, the RNA molecule is a messenger RNA (mRNA) molecule. In some embodiments, (a) the plurality of nucleic acid barcoded molecules further comprise an oligo(dT) sequence; (b) the engineered nucleic acid processing enzyme reverse transcribes the mRNA molecule into a complementary DNA (cDNA) molecule using the mRNA hybridized to the oligo(dT) sequence of the nucleic acid barcoded molecules as a template, and (c) the nucleic acid binding domain binds and stabilizes the mRNA-oligo(dT) hybrid during the reverse transcription, thereby generating a complementary DNA molecule comprising the barcode sequence.

In some embodiments, the engineered nucleic acid processing enzyme further amplifies the complementary DNA molecule comprising the barcode sequence, thereby generating an amplified DNA product comprising the barcode sequence, or complements thereof.

In some embodiments of the nucleic acid extension method disclosed herein, (a) the method further comprises a second nucleic acid molecule comprising an oligo(dT) sequence; (b) the plurality of nucleic acid barcoded molecules further comprise an oligo(dT) sequence; and (c) the nucleic acid binding domain of the engineered nucleic acid processing enzyme binds and stabilizes the mRNA-Oligo(dT) hybrid, while the polymerase domain of the engineered nucleic acid processing enzyme reverse transcribes the mRNA molecule using the second nucleic acid molecule comprising the oligo(dT) sequence, thereby generating a complementary DNA molecule.

In some embodiments, the engineered nucleic acid processing enzyme further amplifies the complementary DNA molecule using the plurality of nucleic acid barcoded molecules, thereby generating an amplified DNA product comprising a barcode sequence. In some embodiments, (a) the plurality of nucleic acid barcoded molecules are attached to a support; and (b) the support is selected from the group consisting of an array, a bead, a gel bead, a microparticle, and a polymer.

In some embodiments of the nucleic acid extension method disclosed herein, the polymerase comprises the amino acid sequence set forth in SEQ ID NO: 1 or an amino acid sequence having at least 90% sequence identity to the amino acid sequence set forth in SEQ ID NO: 1. In some embodiments, the polymerase comprises an amino acid sequence having: (a) at least 90%, at least 91%, at least 92%, at least 93%, at least 94%, at least 95%, at least 96%, at least 97%, at least 98%, or at least 99% sequence identity to the amino acid sequence of SEQ ID NO: 1; (b) at least 95% identity to the amino acid sequence of SEQ ID NO: 1; (c) at least 97% identity to the amino acid sequence of SEQ ID NO: 1; (d) at least about 10, at least about 15, or at least about 20 substitutions in the amino acid sequence of SEQ ID NO: 1; or (e) at least 97% identity to the amino acid sequence of SEQ ID NO: 1 and at least about 15 substitutions in the amino acid sequence of SEQ ID NO: 1. In some embodiments, the polymerase comprises an amino acid sequence having 90%, 91%, 92%, 93%, 94%, 95%, 96%, 97%, 98%, or 99% sequence identity to the amino acid sequence of SEQ ID NO: 1.

In some embodiments, (a) the polymerase domain comprises a substitution at positions 141 and 143 of SEQ ID NO: 1 or 2; (b) the polymerase domain comprises a substitution at position 141 of SEQ ID NOs: 2; (c) the engineered nucleic acid processing enzyme lacks proofreading activity; or (d) the engineered nucleic acid processing enzyme comprises the amino acid sequence of SEQ ID NO: 3.

In some embodiments of the engineered nucleic acid processing enzyme described herein, the polymerase domain comprises: (a) an aspartic acid substitution at position 141; (b) a glutamic acid substitution at position 143; (c) an alanine substitution at position 485; (d) a valine substitution at position 93; (e) an arginine substitution at position 97; (f) a tyrosine substitution at position 384; (g) a valine substitution at position 389; (h) a phenylalanine substitution at position 493; (i) a phenylalanine substitution at position 587; (j) a glutamic acid substitution at position 664; (k) a glycine substitution at position; (l) a tryptophan substitution at position 768; (m) an isoleucine substitution at position 2; (n) an isoleucine substitution at position 38; (o) a lysine substitution at position 118; (p) a methionine substitution at position 137; (q) an arginine substitution at position 381; (r) a lysine substitution at position 466; (s) a tyrosine substitution at position 514; (t) an isoleucine substitution at position 521; and/or (u) an asparagine substitution at position 735 in SEQ ID NO: 1.

In some embodiments, the polymerase domain comprises: (a) an aspartic acid to alanine substitution at position 141 (D141A); (b) a glutamic acid to alanine substitution at position 143 (E143A); (c) an alanine to leucine substitution at position 485 (A485L); (d) a valine to glutamine substitution at position 93 (V93Q); (e) an arginine to methionine substitution at position 97 (R97M); (f) a tyrosine to histidine substitution at position 384 (Y384H); (g) a valine to isoleucine substitution at position 389 (V389I); (h) a phenylalanine to leucine substitution at position 493 (F493L); (i) a phenylalanine to leucine substitution at position 587 (F587L); (j) a glutamic acid to lysine substitution at position 664 (E664K); (k) a glycine to valine substitution at position (G711V); (l) a tryptophan to arginine substitution at position 768 (W768R); (m) an isoleucine to valine substitution at position 2 (12V); (n) an isoleucine to leucine substitution at position 38 (I38L); (o) a lysine to isoleucine substitution at position 118 (K118I); (p) a methionine to leucine substitution at position 137 (M137L); (q) an arginine to histidine substitution at position 381 (R381H); (r) a lysine to arginine substitution at position 466 (K466R); (s) a tyrosine to isoleucine substitution at position 514 (T514I); (t) an isoleucine to leucine substitution at position 521 (I521L); and/or (u) an asparagine to lysine substitution at position 735 (N735K) in SEQ ID NO: 1.

In some embodiments, the polymerase domain comprises a combination of: (a) R97M, D141A, E143A, Y384H, V389I; Y493L; F587L, E664K, G711V, and W768R substitutions in SEQ ID NO: 1; (b) I2V, I38L, R97M, K118I, M137L, E143A, R381H; Y384H, V389I, K466R, F493L, T514I, I521L, F587L, E664K, G711V, N735K, and W768R substitutions in SEQ ID NO: 1; (c) I2V, I38L, R97M, K118I, M137L, D141A, E143A, R381H, Y384H, V389I, K466R, F493L, T514I, I521L, F587L, E664K, G711V, N735K, and W768R substitutions in SEQ ID NO: 1; or (d) I2V, I38L, V93Q, R97M, K118I, M137L, D141A, E143A, R381H, Y384H, A485L, V389I, K466R, F493L, T514I, I521L, F587L, E664K, G711V, N735K, and W768R substitutions in SEQ ID NO: 1.

In some embodiments of the nucleic acid extension method disclosed herein, the polymerase domain comprises the amino acid sequence set forth in SEQ ID NO. 2. In some embodiments, the polymerase domain comprises the amino acid sequence of SEQ ID NO: 3.

In some embodiments, the nucleic acid binding domain comprises: (a) a single stranded DNA binding protein; (b) a double stranded DNA binding protein; (c) a single stranded RNA binding protein; (d) a double stranded RNA binding protein; (e) a continuous RNA-DNA hybrid binding protein; or (f) a discontinuous RNA-DNA hybrid binding protein.

In some embodiments, the nucleic acid binding domain comprises a nucleic acid binding protein selected from the group consisting of a histone-like protein, an archaeal basic nucleic acid binding protein, a basic DNA binding domain, HMf-like protein, Proliferating cell nuclear antigen (PCNA), HU-like protein, HU-family DNA binding protein, Sm-like protein domain; HU, sto7, Sso7d, Sac7d, and Sac7e.

In some embodiments, (a) the nucleic acid binding domain comprises a histone-like protein; (b) the nucleic acid binding domain is a PCNA, optionally a T. kodakarensis PCNA or (c) the nucleic acid binding domain comprises a polynucleotide encoding the amino acid sequence of SEQ ID NO: 16 or a sequence having at least 90% sequence identity to the amino acid sequence of SEQ ID NO: 16. In some embodiments, the nucleic acid binding domain comprises a bacterial histone-like protein or a bacterial HU-family DNA binding protein. In some embodiments, the nucleic acid binding domain is a Thermus thermophile HU-family DNA binding protein. In some embodiments, the nucleic acid binding domain binds a DNA and RNA hybrid complex, wherein the DNA-RNA hybrid is continuous or discontinuous.

In some embodiments of the nucleic acid extension method disclosed herein, the nucleic acid binding protein comprises an amino acid sequence having at least 90%, at least 91%, at least 92%, at least 93%, at least 94%, at least 95%, at least 96%, at least 97%, at least 98%, or at least 99% sequence identity to the amino acid sequence of SEQ ID NO: 6. In some embodiments of the nucleic acid extension method disclosed herein, the nucleic acid binding protein comprises an amino acid sequence having 90%, 91%, 92%, 93%, 94%, 95%, 96%, 97%, 98%, or 99% sequence identity to the amino acid sequence of SEQ ID NO: 6. In some embodiments, the nucleic acid binding domain comprises an amino acid sequence set forth in SEQ ID NO: 6 or an amino acid sequence having at least 90% sequence identity to the amino acid sequence set forth in SEQ ID NO: 6. In some embodiments, the engineered nucleic acid processing enzyme comprises an amino acid sequence of SEQ ID NO: 5; or an amino acid sequence having at least 90% sequence identity to the amino acid sequence set forth in SEQ ID NO: 5. In some embodiments, the engineered nucleic acid processing enzyme comprises an amino acid sequence of SEQ ID NO: 4; or an amino acid sequence having at least 90% sequence identity to the amino acid sequence of SEQ ID NO: 4.

In some embodiments of the nucleic acid extension method disclosed herein, the engineered nucleic acid processing enzyme further comprises a tag protein selected from the group consisting of an affinity tag, a fluorescent tag, or an expression and/or solubility enhancement tag. In some embodiments, the tag is selected from hexahistidine tag (his-tag), small ubiquitin-like modifier tag (SUMO), aVariFlex C-Terminal solubility enhancement tag, a short peptide C-terminal tag, Thioredoxin (Trx) tag, aVariFlex C-Terminal solubility enhancement tag, Solubility-enhancer peptide sequences (SET) tag, IgG domain B1 of Protein G (GB1) tag, IgG repeat domain ZZ of Protein A (ZZ) tag, Solubility enhancing Ubiquitous Tag (SNUT tag), Seventeen kilodalton protein (Skp tag), Phage T7 protein kinase (T7PK) tag, E. coli secreted protein A (EspA) tag, Monomeric bacteriophage T7 0.3 protein (Orc protein) (Mocr) tag, E. coli trypsin inhibitor (Ecotin) tag, Calcium-binding protein (CaBP) tag, Stress-responsive arsenate reductase (ArsC) tag, N-terminal fragment of translation initiation factor IF2 (IF2-domain I) tag, N-terminal fragment of translation initiation factor IF2 (Expressivity) tag, Fasciola hepatica 8-kDa antigen tag (Fh8), Glutathione-S-transferase (GST) tag, maltose-binding protein tag (MBP), FLAg tag peptide (FLAG), streptavidin binding peptide tag (Strep-II; strep), calmodulin-binding protein tag (CBP), mutated dehalogenase tag (HaloTag), staphylococcal Protein A (Protein A), intein mediated purification with the chitin-binding domain (IMPACT (CBD)), cellulose-binding module (CBM), dockerin domain of Clostridium josui tag (Dock), fungal avidin-like protein (Tamavidin).

In some embodiments, the tag is an affinity tag selected from hexahistidine tag (his-tag), Fasciola hepatica 8-kDa antigen tag (Fh8), Glutathione-S-transferase (GST) tag, maltose-binding protein tag (MBP), FLAg tag peptide (FLAG), streptavidin binding peptide tag (Strep-II), calmodulin-binding protein tag (CBP), mutated dehalogenase tag (HaloTag), staphylococcal Protein A (Protein A), intein mediated purification with the chitin-binding domain (IMPACT (CBD)), cellulose-binding module (CBM), dockerin domain of Clostridium josui tag (Dock), fungal avidin-like protein (Tamavidin).

In some embodiments, the engineered nucleic acid processing enzyme comprises: (a) an hexahistidine tag (his-tag); or (b) an amino acid sequence of SEQ ID NO: 9; or an amino acid sequence having at least 90% sequence identity to the amino acid sequence of SEQ ID NO: 9.

In some embodiments, the engineered nucleic acid processing enzyme comprises a solubility enhancer tag selected from the group consisting of a SUMO tag, a GST tag, a Trx tag, aVariFlex C-Terminal solubility enhancement tag, a short peptide C-terminal tag, an Fh8 tag, MBP tag, SET tag, GB1 tag, ZZ tag, HaloTag, SNUT tag, Skp tag, T7PK tag, EspA tag, Mocr tag, Ecotin tag, CaBO tag, ArsC tag, IF2-domain I tag, Expressivity tag, RpoA, tag, SlyD, tag, Tsf tag, RpoS tag, PotD tag, Crr tag, msyB tag, yigD tag, and rpoD tag. In some embodiments, the engineered nucleic acid processing enzyme comprises (a) a short peptide C-terminal tag; (b) an amino acid sequence of SEQ ID NO: 10; or (c) an amino acid sequence having at least 90% sequence identity to the amino acid sequence of SEQ ID NO: 10.

In some embodiments, the tag further comprises: (a) an endoprotein cleavage sequence; (b) a cleavage sequence recognized by an endoprotein selected from the group consisting of alanine carboxypeptidase, Armillaria mellea astacin, bacterial leucyl aminopeptidase, cancer procoagulant, cathepsin B, clostripain, cytosol alanyl aminopeptidase, elastase, endoproteinase Arg-C, enterokinase (EnTK), gastricsin, gelatinase, Gly-X carboxypeptidase, glycyl endopeptidase, human rhinovirus 3C protease, hypodermin C, Iga-specific serine endopeptidase, leucyl aminopeptidase, leucyl endopeptidase, lysC, lysosomal pro-X carboxypeptidase, lysyl aminopeptidase, methionyl aminopeptidase, myxobacter, nardilysin, pancreatic endopeptidase E, picornain 2A, picornain 3C, proendopeptidase, prolyl aminopeptidase, proprotein convertase I, proprotein convertase II, russellysin, saccharopepsin, semenogelase, T-plasminogen activator, thrombin (Thr), tissue kallikrein, tobacco etch virus (TEV), togavirin, tryptophanyl aminopeptidase, U-plasminogen activator, V8, venombin A, venombin AB, factor Xa (Xa), and Xaa-pro aminopeptidase; or (c) an endoprotein cleavage sequence comprising the amino acid sequence of SEQ ID NO: 11, SEQ ID NO: 12, SEQ ID NO: 13, SEQ ID NO: 14, or SEQ ID NO: 15.

In some embodiments, the engineered nucleic acid processing enzyme: (a) is thermophilic; (b) is resistant to thermal inactivation when compared to a wild-type polymerase; (c) is resistant to thermal inactivation at a temperature from about 53° C. to about 75° C.; from about 55° C. to about 75° C.; from about 60° C. to about 75° C.; from about 53° C. to about 68° C.; from about 55° C. to about 68° C.; from about 45° C. to about 68° C.; or from about 50° C. to about 68° C.; or (d) is resistant to thermal inactivation at a temperature of about 68° C.

In some embodiments, the engineered nucleic acid processing enzyme possesses enhanced half-life when compared to a wild-type polymerase at a temperature from about 53° C. to about 75° C.; from about 55° C. to about 75° C.; from about 60° C. to about 75° C.; from about 53° C. to about 68° C.; from about 55° C. to about 68° C.; from about 45° C. to about 68° C.; or from about 50° C. to about 68° C.

In some embodiments of the nucleic acid extension method disclosed herein, the engineered nucleic acid processing enzyme possesses one or more of the following characteristics when compared to a wild-type polymerase: (a) increased thermostability; (b) increased thermoreactivity; (c) increased resistance to reverse transcriptase inhibitors; (d) increased ability to reverse transcribe difficult templates; (e) increased speed; (f) increased processivity; (g) increased specificity; (h) enhanced polymerization activity; or (i) increased sensitivity.

In some embodiments the increase in thermoreactivity, resistance to reverse transcriptase inhibitors, ability to reverse transcribe difficult templates, speed, processivity, specificity, or sensitivity is about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 90%, or about 100% as compared to the wild-type polymerase. In some embodiments, the polymerization activity is enhanced by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 90%, or about 100% as compared to the wild-type polymerase.

In some embodiments of the nucleic acid extension method disclosed herein, the chimeric polymerase enzyme has a dual DNA and RNA polymerase activity, or an RNA reverse transcriptase activity. In some embodiments, the chimeric polymerize enzyme reverse transcribes a RNA molecule having at least about 100, at least about 200, at least about 300, at least about 400, at least about 500, at least about 600, at least about 700, at least about 800, at least about 900, or at least about 1000 nucleotides. In some embodiments, the chimeric polymerize enzyme reverse transcribes a RNA molecule that is at least about 1 kb, at least about 2 kb, at least about 3 kb, at least about 4 kb, at least about 5 kb, at least about 6 kb, at least about 7 kb, at least about 8 kb, at least about 9 kb, at least about 10 kb, at least about 11 kb, at least about 12 kb, at least about 13 kb, at least about 14 kb, or at least about 15 kb. In one embodiment, the chimeric polymerize enzyme reverse transcribes a RNA molecule that is at least about 7 kb or at least about 8 kb.

In some embodiments, the target nucleic acid molecule is further contacted with a sliding clamp molecule. In some embodiments, the sliding clamp molecule is an archea, eucarya, or a bacteriophage sliding clamp protein. In some embodiments, the sliding clamp protein is selected from E. coli polymerase β subunit; T4 bacteriophage gp45, T. gorgonarius PCNA, or T. kodakarensis PCNA. In some embodiments, the sliding clamp protein is T kodakarensis PCNA.

One aspect of the present disclosure provides a kit comprising the engineered thermostable reverse transcriptase disclosed herein. In some embodiments, the kit further comprises one or more of a vector, a nucleotide, a buffer, a salt, and/or instructions.

Both the foregoing summary and the following description of the drawings and detailed description are exemplary and explanatory. They are intended to provide further details of the disclosure, but are not to be construed as limiting. Other objects, advantages, and novel features will be readily apparent to those skilled in the art from the following detailed description of the disclosure.

### BRIEF DESCRIPTION OF THE DRAWINGS

FIG. 1A shows a chromatograph of a reverse transcribed product generated using an engineered Moloney Murine Leukemia Virus (MMLV) reverse-transcriptase (MMLV RT) enzyme variant at 53° C. demonstrating that the MMLV RT variant reverse transcribed a target mRNA and generated an amplification product at 53° C. (at arrow).

FIG. 1B shows a chromatograph of a reverse transcribed product generated using an engineered T. gorgonarius reverse-transcriptase lacking proof reading activity (3′-5′ exonuclease) (TgoRTxo) at 53° C., demonstrating that TgoRTxo reverse transcribed an mRNA and generated an amplification product at 53° C. (at arrow).

FIG. 2A shows a chromatograph of a reverse transcribed product generated using the MMLV RT variant enzyme of FIG. 1A at 68° C., demonstrating that the MMLV RT variant enzyme could not reverse transcribe an mRNA and did not generate an amplification product at 68° C. (at arrow).

FIG. 2B shows a chromatograph of a reverse transcribed product generated using the TgoRTxo enzyme of FIG. 1B at 68° C., demonstrating that the TgoRTxo was able to reverse transcribe a target mRNA and generate an amplification product at 68° C. (at arrow).

FIG. 3 shows a sequence alignment between wild type T. gorgonarius DNA polymerase (TgoPol) and wild type T. kodakarensis polymerase (KodPol), demonstrating that wild type T. gorgonarius DNA polymerase (TgoPol) shares some similarity to wild type T. kodakarensis polymerase (KodPol).

FIG. 4 shows a bar graph illustrating the efficiency of TgoRTxo reverse transcription reactions in the presence of varying concentrations of T. kodakarensis proliferating cell nuclear antigen (KPCNA).

FIG. 5 shows a schematic diagram of a generalized capture probe used in spatial transcriptomics and single cell transcriptomic analyses, exemplary applications where the engineered thermostable reverse transcriptase of the invention could be used to extend a capture probe using a captured target nucleic acid as a template, thereby generating a cDNA product.

### DETAILED DESCRIPTION

It is to be appreciated that certain aspects, modes, embodiments, variations and features of the present methods are described below in various levels of detail in order to provide a substantial understanding of the present technology.

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

### I. Overview

Despite the critical role played by reverse transcriptases in molecular biology, inherent limitations exist with known reverse transcriptases that limit their widespread in vitro use. In particular, natural reverse transcriptase enzymes are mesophilic and cannot be used at temperatures above 37° C. The mesophilic nature of RT is problematic for in vitro amplification reactions of RNAs because RNAs tend to adopt stable secondary structures at lower temperatures. Such secondary structures can form, for example, when regions of RNA molecules have sufficient complementarity to hybridize and form double stranded RNA. These secondary structures affect the efficiency of the reverse transcription reactions at low to moderate temperatures. Generally, the formation of RNA secondary structures can be prevented by raising the temperature of solutions/reactions which contain the RNA molecules. However, natural RT enzymes lose their activity at temperatures above 37° C., and some engineered RTs lose their activity at temperatures above 53° C.

Furthermore, this narrow temperature range of activity makes it harder to combine reverse transcription reaction (RTx) with cDNA amplification (PCR), for example in the same amplification reaction and in the absence of a second polymerase. A technique used in the art to permit RTx and PCR in a single reaction can include the use of template switching oligonucleotides, and the addition of a reverse transcriptase and a DNA polymerase in the same reaction. The use of a second polymerase in the same reaction mixture as the RT reaction has consequences for the quality of the data generated. For instance, it was observed while performing experiments related to the present disclosure that the sensitivity and specificity of these reactions were generally weaker and more time consuming when two polymerases were combined in the same reaction.

In contrast to RT enzymes, DNA polymerases have high fidelity and high thermostability. In particular, archaeal Family-B polymerases (polB) have been widely adopted in modern molecular biology due to their hyperthermostability, processivity, and fidelity. However, Family B polymerase enzymes show little to no activity on RNA templates.

To overcome these limitations, described herein is an engineered Thermococcus gorgonarius polymerase (Tgo polymerase) exhibiting a combination of reverse transcriptase activity and high thermostability. In addition, the engineered Tgo reverse transcriptase described herein is more efficient and processive than a wild-type Tgo polymerase. The engineered reverse transcriptase of the present disclosure can be used in a single amplification reaction to generate a nucleic acid amplification product (DNA) by first generating a cDNA from mRNA and then amplifying that cDNA using the single engineered reverse transcriptase polymerase enzyme of the present disclosure. This one-step reaction has many advantages. For example, a thermophilic reverse transcriptase enzyme with dual reverse transcriptase and DNA polymerase activity would: (1) render unnecessary the use of template switching oligonucleotides; (2) reduce the dependence on template switching for amplification reactions as found in spatial array and single cell transcriptomics assay, and (3) simplify and expedite any RT-PCR reactions as known in the art.

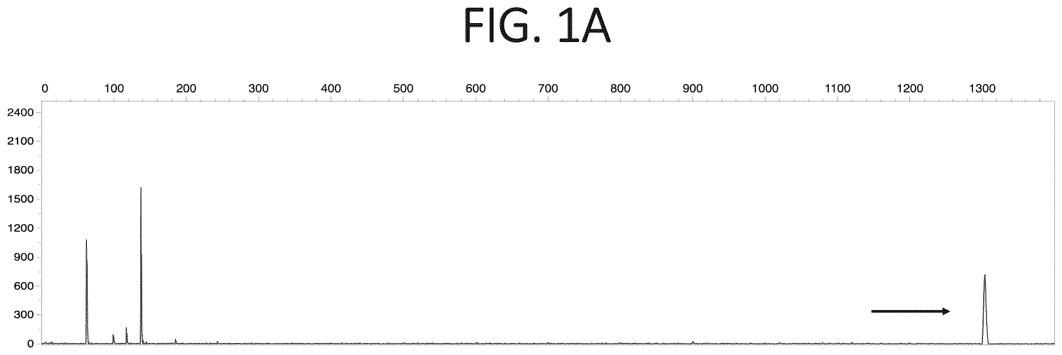

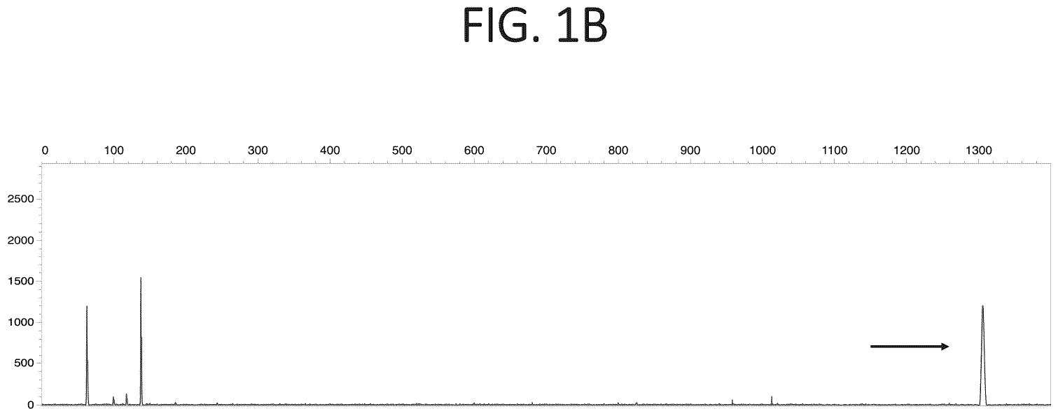

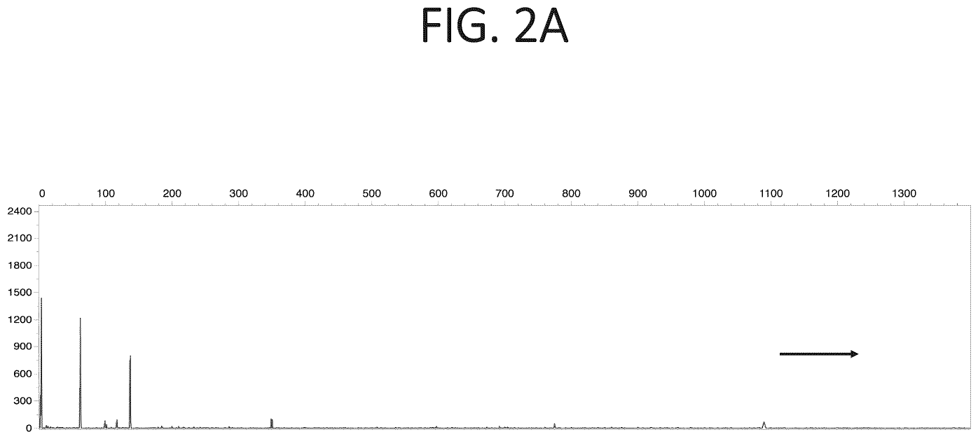

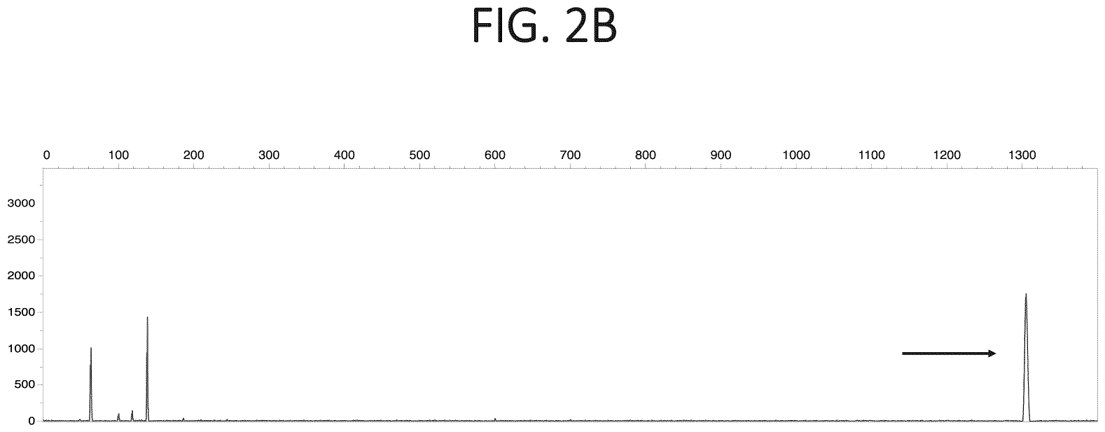

Accordingly, the present disclosure provides engineered recombinant archeal Family-B polymerases that have the fidelity and thermostability of known DNA polymerases in combination with a reverse transcriptase activity. The family B polymerase was genetically engineered, via mutagenesis, to have the properties of a reverse transcriptase, while maintaining the DNA polymerase activity. As shown in FIG. 1B and FIG. 2B, the engineered reverse transcriptase of the present disclosure functions as well or better at product generation at moderate (53° C.) as at high (68° C.) temperatures, the arrows indicating the expected size of the 1300 nt RNA molecule that was reverse transcribed at the two different temperatures. As previously mentioned, companion experiments demonstrated that an MMLV RT enzyme variant was able to generate a product at 53° C. (FIG. 1A), but it was not able to function to generate a product at 68° C. (FIG. 2A). In fact, the relative amount of product generated using the MMLV RT enzyme is about half (approximately 600) when compared to the TgoRTxo product generation (approximately 1200). In addition, the TgoRTxo product generation was increased at 68° C. over that seen at 53° C.

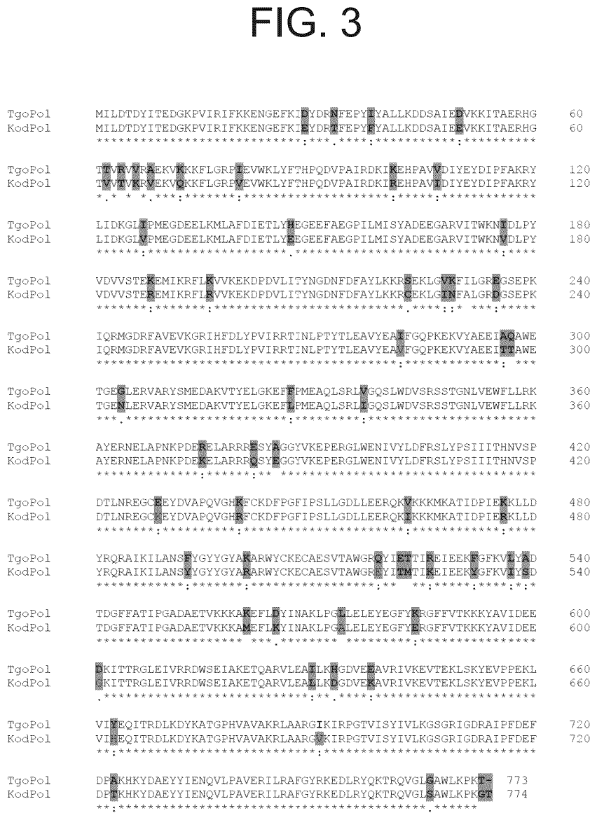

The engineered thermostable enzymes of the present disclosure are novel over those known in the art for a number of reasons. First, the present disclosure started from a polymerase enzyme that has not been characterized in the art. As shown in FIG. 3, the amino acid sequence of the wild type T. gorgonarius DNA polymerase (TgoPol) is only approximately 92.63% identical to the amino acid sequence of a wild type T. kodakarensis polymerase (KodPol), which has previously been mutagenized and characterized in the art. See e.g., Ellefson et al. Science, 352(6293):1590-3 (2016). Furthermore, amino acids that are important for the properties of the engineered thermostable reverse transcriptase of the present disclosure are not conserved.

To further improve the biophysical properties of the disclosed engineered thermostable reverse transcriptase, the thermostable reverse transcriptase was engineered as a recombinant fusion protein comprising a first domain with the engineered thermostable reverse transcriptase polymerase domain, and a second domain, 3′ to the first domain, that comprises a nucleic acid binding domain encoding a HU family protein. This second domain further enhanced the fidelity and processivity of the engineered (i.e., recombinant) reverse transcriptase. In particular, the second domain encodes a HU protein. HU is a bacterial protein that exhibits a dual specific DNA and RNA binding activity. HU binds to double-stranded DNA, double-stranded RNA, and linear DNA-RNA duplexes with a similar low affinity. However, HU is also able to bind all forms of nucleic acids, including DNA-RNA hybrids. In fact, HU binding to discontinuous DNA-RNA structures is much stronger than its binding to DNA and RNA duplexes. HU showed higher affinity for DNA-RNA hybrids than for RNA or DNA duplexes. Because of HU's ability to bind all forms of nucleic acids, its fusion to the thermostable reverse transcriptase enzyme provides a novel and non-obvious recombinant thermostable polymerase. The engineered thermostable reverse transcriptase of the present disclosure is a novel tool for overcoming the limitations associated with sequencing RNA templates and or using RNA templates in a single cell analysis system, or in spatial array and single cell transcriptomics assays as disclosed herein. In addition to its nucleic acid-binding ability, it was surprisingly found that HU helps stabilize nucleic acids against thermal denaturation. While the art discloses conjugating various DNA binding domains to a polymerase, the art does not teach or suggest using HU to improve the efficiency and processitivity of reverse transcriptases.

In one aspect, the present disclosure provides an engineered nucleic acid processing enzyme comprising a first domain comprising a polymerase domain, operably linked to a second domain 3′ to the first domain. In some embodiments, the polymerase domain comprises an amino acid sequence of an engineered Thermococcus gorgonarius polymerase (Tgo polymerase). In some embodiments, the polymerase comprises the amino acid sequence of SEQ ID NO: 1 or an amino acid sequence having at least about 90% sequence identity to the amino acid sequence of SEQ ID NO: 1. In other aspects, the polymerase comprises a sequence having at least about 91%, about 92%, about 93%, about 94%, about 95%, about 96%, about 97%, about 98%, about 99% or about 100% sequence identity to the amino acid sequence of SEQ ID NO: 1. In other aspects, the polymerase comprises a sequence having 91%, 92%, 93%, 94%, 95%, 96%, 97%, 98%, 99% or 100% sequence identity to the amino acid sequence of SEQ ID NO: 1. In some embodiments, the second domain of the engineered nucleic acid processing enzyme comprises a nucleic acid binding domain. In some embodiments, the nucleic acid binding domain is a nucleic acid binding protein or a derivative or portion thereof.

In another aspect, the present disclosure provides an isolated nucleic acid molecule encoding an engineered nucleic acid processing enzyme as disclosed herein. In another aspect, the present disclosure provides an expression vector comprising the isolated nucleic acid and a host cell transfected with the expression vector. In some aspects, the expression vector further comprises a protein purification tag, for example a polyhistidine purification tag either 3′ or 5′ of the engineered thermostable reverse transcriptase sequences. In additional aspects, the expression vector may further comprise a sequence that is useful in enhancing the secretion, expression, etc. of the engineered thermostable reverse transcriptase thereby increasing the yield of the engineered nucleic acid processing enzyme in the chosen protein expression system. For example, a SUMO-tag can be used to increase the expression and solubility of a desired recombinant protein. The present disclosure is not limited to the type of additional attributes that might be associated with any expression vector or expression protein system for producing the engineered thermostable reverse transcriptase protein as described herein.

In one aspect, the present disclosure provides a method of using the engineered nucleic acid processing enzyme described herein, the method comprising contacting the engineered nucleic acid processing enzyme with a nucleic acid template under suitable conditions to produce a polymerized nucleic acid product. The nucleic acid template can be an RNA, a DNA, or a nucleic acid comprising an unnatural nucleotide, or a combination thereof.

In another aspect, the present disclosure provides a nucleic acid extension method comprising contacting a target nucleic acid molecule with an engineered nucleic acid processing enzyme, as described herein, and a plurality of nucleic acid barcoded molecules comprising a barcode sequence, and incubating the target nucleic acid, engineered nucleic acid processing enzyme and barcoded molecules under conditions in which the target nucleic acid is extended by the engineered nucleic acid processing enzyme. In some embodiments, the engineered nucleic acid processing enzyme comprises a first domain comprising a polymerase domain, and a second domain operably linked to the first domain, where the second domain comprises a nucleic acid binding domain. The polymerase domain can comprise an amino acid sequence of an engineered Thermococcus gorgonarius polymerase (Tgo polymerase). In the nucleic acid extension method, the polymerase domain extends one of the plurality of nucleic acid barcoded molecules that is hybridized to the target nucleic acid molecule; and the nucleic acid binding domain binds and stabilizes the target nucleic acid molecule.

### II. An Engineered Thermophilic Reverse Transcriptase

A. Polymerases Suitable for Engineering

In one aspect, the present disclosure provides an engineered nucleic acid processing enzyme comprising a first domain comprising a polymerase domain operably linked to a second domain. In some embodiments, the polymerase domain comprises an amino acid sequence of an engineered Thermococcus gorgonarius polymerase (Tgo polymerase). In some embodiments, the polymerase comprises the amino acid sequence of SEQ ID NO: 1 or an amino acid sequence having at least 90% sequence identity to the amino acid sequence of SEQ ID NO: 1. In some embodiments, the second domain of the engineered nucleic acid processing enzyme comprises a nucleic acid binding domain. In some embodiments, the nucleic acid binding domain is from a nucleic acid binding protein or is a nucleic acid binding protein.

Monomeric archaeal Family-B polymerases (polB) have been widely adopted in modern molecular biology due to their hyperthermostability, processivity, and fidelity. Accordingly, polymerases suitable for engineering a thermostable reverse transcriptase enzyme as described herein are not limited to a Thermococcus gorgonarius polymerase. In some embodiments, polymerases suitable for engineering a reverse transcriptase of the present disclosure include, but are not limited to archaeal, bacterial, and eukaryotic polymerases. Polymerases include both DNA-dependent polymerases and RNA-dependent polymerases such as reverse transcriptases. At least five families of DNA-dependent DNA polymerases are known, although most fall into families A, B and C. There is little or no sequence similarity among the various families. Most family A polymerases are single chain proteins that can contain multiple enzymatic functions including polymerase activity, 3′ to 5′ exonuclease activity and 5′ to 3′ exonuclease activity. Family B polymerases typically have a single catalytic domain with a polymerase, and 3′ to 5′ exonuclease activity, as well as accessory factors. Family C polymerases are typically multi-subunit proteins with polymerizing activity and 3′ to 5′ exonuclease activity.

In some embodiments, the polymerase of the present disclosure is a B-type family DNA polymerase. B-type Family DNA polymerases include, but are not limited to any DNA polymerase that is classified as a member of the Family B DNA polymerases. The Family B classification is based on structural similarity to E. coli DNA polymerase II and is also based on the presence of known and conserved regions referred to as motif A and motif B of the family B polymerases. B-type family polymerases include bacterial and bacteriophage polymerases. In some embodiments, the B-type family polymerase is E. coli DNA polymerase II; PRD 1 DNA polymerase; phi29 DNA polymerase; M2 DNA polymerase; and T4 DNA polymerase. In some embodiments, the B-type family polymerase is an archaeal DNA polymerases such as Thermococcus litoralis DNA polymerase (Vent); Pyrococcus furiosus DNA polymerase; Sulfolobus solfataricus DNA polymerase; Thermococcus gorgonarius DNA polymerase (Tgo pol); Pyrodictium occultum DNA polymerase; Methanococcus voltae DNA polymerase; Thermococcus species TY; T. kodakarensis polymerase (KodPol); Sulfolobus acidocaldarius DNA polymerase; Thermococcus species 9° N-7 (Therminator™); or Thermococcus species 9° N.

In some embodiments, the polymerase is an Eukaryotic B-type family DNA polymerases selected from the group consisting of DNA polymerase alpha; Human DNA polymerase (alpha); S. cerevisiae DNA polymerase (alpha); S. pombe DNA polymerase I (alpha); Drosophila melanogaster DNA polymerase (alpha); Trypanosoma brucei DNA polymerase (alpha); DNA polymerase delta; Human DNA polymerase (delta); Bovine DNA polymerase (delta); S. cerevisiae DNA polymerase III (delta); S. pombe DNA polymerase III (delta); and Plasmodium falciparum DNA polymerase (delta).

DNA polymerases have a common overall structure that has been likened to a human right hand, with fingers, thumb, and palm subdomains. The palm subdomain contains motif A which in turn contains a catalytically active aspartic acid residue. In native DNA polymerases, motif A begins at an anti-parallel β-strand containing predominantly hydrophobic residues and is followed by a turn and an α-helix. In native DNA polymerases, motif A interacts with a next correct nucleotide via coordination with divalent metal ions that participate in the polymerization reaction. Motif B contains an alpha-helix with positive charges. Further characteristics of motif A and motif B are known in the art, for example, as set forth in Delarue et al., Protein Eng., 3: 461-467 (1990); Shinkai et al., J. Biol. Chem., 276: 18836-18842 (2001), and Steitz, T. A., J. Biol. Chem., 274:17395-17398 (1999).

In some embodiments, the polymerase is a family B polymerase comprising a motif A and a motif B conserved regions. The terms “motif A” and “motif B” are intended to be used in accordance with their known meaning in the art. The terms are used to refer to regions of structural homology in the nucleotide binding sites of B family and other polymerases. Motif A and motif B are conserved regions among polymerases involved in nucleotide binding and substrate specificity. In some embodiments, motif A refers specifically to amino acids 408-410 of SEQ ID NO: 1 (Wild type Tgo Pol), or a motif that includes amino acids 408-410 of SEQ ID NO: 1. In some embodiments, motif B refers specifically to amino acids 484-486 of SEQ ID NO: 1, or to the motif that includes amino acids 484-486 SEQ ID NO: 1. Functionally equivalent or homologous “motif A” and “motif B” regions of polymerases other than the ones described herein can be identified on the basis of amino acid sequence alignment and/or molecular modelling. Sequence alignments may be compiled using any of the standard alignment tools known in the art, such as for example BLAST or CLUSTAL W.

Other polymerases that can be engineered include, for example, those that are members of families identified as A, C, D, X, Y, and RT. The RT (reverse transcriptase) family of DNA polymerases includes, but is not limited to retrovirus reverse transcriptases and eukaryotic telomerases. Exemplary RNA polymerases include, but are not limited to, viral RNA polymerases such as T7 RNA polymerase; eukaryotic RNA polymerases, such as RNA polymerase I, RNA polymerase II, RNA polymerase III, RNA polymerase IV, and RNA polymerase V; and archaea RNA polymerase. Motif A is present in RNA polymerases and can be modified at specified positions to generate DNA polymerases. Conversely, DNA polymerase can be modified as disclosed herein to engineer an enzyme with RT activity.

In some embodiments, the polymerase domain of the engineered nucleic acid processing enzyme described herein comprises an amino acid sequence having at least about 90%, at least about 91%, at least about 92%, at least about 93%, at least about 94%, at least about 95%, at least about 96%, at least about 97%, at least about 98%, or at least about 99% sequence identity to the amino acid sequence of SEQ ID NO: 1. In some embodiments, the polymerase domain of the engineered nucleic acid processing enzyme described herein comprises an amino acid sequence having 90%, 91%, 92%, 93%, 94%, 95%, 96%, 97%, 98%, or 99% sequence identity to the amino acid sequence of SEQ ID NO: 1. In one embodiment, the polymerase domain of the engineered nucleic acid processing enzyme described herein comprises an amino acid sequence having at least about 95% identity to the amino acid sequence of SEQ ID NO: 1. In another embodiment, the polymerase domain of the engineered nucleic acid processing enzyme described herein comprises an amino acid sequence having at least about 97% identity to the amino acid sequence of SEQ ID NO: 1. In yet another embodiment, the polymerase domain of the engineered nucleic acid processing enzyme described herein comprises an amino acid sequence having at least about 10, about 11, about 12, about 13, about 14, about 15, about 16, about 17, about 18, about 19 or about 20 substitutions in the amino acid sequence of SEQ ID NO: 1. In yet another embodiment, the polymerase domain of the engineered nucleic acid processing enzyme described herein comprises an amino acid sequence having at least about 97% identity to the amino acid sequence of SEQ ID NO: 1 and/or at least about 15 substitutions in the amino acid sequence of SEQ ID NO: 1.

The percent sequence identity, in the context of two or more nucleic acid or polypeptide sequences, refers to the number of residues or bases that are the same for a given alignment of two polypeptide or nucleic acid sequences. Sequences sharing a specified percentage of nucleotides or amino acid residues, respectively, that are the same, when compared and aligned for a given parameter such as maximum correspondence, as measured using one of the sequence comparison algorithms described below (or other algorithms available to persons of skill) or by visual inspection.

By convention, amino acid additions, substitutions, and deletions within an aligned reference sequence are all differences that may reduce the percent identity depending upon the parameters used to assess percent identity. Often, additions, substitutions, and deletions within an aligned reference sequence are evaluated in an equivalent manner. In some cases, length variation between two sequences resulting in one sequence having bases or residues beyond the N- or C-terminus or 5′ or 3′ end of the other sequence are discarded in sequence alignment, such that the aligned region is defined by the ends of the shorter or earlier ending sequence and amino acids extending beyond the N- or C-terminus of a polynucleotide or 5′ or 3′ end of the earlier terminating sequence have no effect on percent identity scoring for aligned regions. For example, by one calculation approach, alignment of a 105 amino acid long polypeptide to a reference sequence 100 amino acids long would have a 100% identity score if the reference sequence fully was contained as a consecutive ungapped segment within the longer polynucleotide with no amino acid differences. Under such an assessment, a single amino acid difference (addition, deletion or substitution) between the two sequences within the 100-amino acid span of the aligned reference sequence would mean the two sequences were 99% identical.

In contrast, “substantially identical,” in the context of two nucleic acids or polypeptides (e.g., DNAs encoding a polymerase, or the amino acid sequence of a polymerase) refers to two or more sequences or subsequences that have at least about 60%, at least about 80%, at least about 90-95%, at least about 97%, at least about 98%, at least about 99% or more nucleotide or amino acid residue identity, when compared and aligned for maximum correspondence, as measured using a sequence comparison algorithm, or by visual inspection. Such “substantially identical” sequences are typically considered to be “homologous,” without reference to actual ancestry. The “substantial identity” exists over a region of the sequences that is at least about 50 residues in length, at least about 100 residues, at least about 150 residues, or over the full length of the two sequences to be compared.

Proteins and/or protein sequences are “homologous” when they are derived, naturally or artificially, from a common ancestral protein or protein sequence. Similarly, nucleic acids and/or nucleic acid sequences are homologous when they are derived, naturally or artificially, from a common ancestral nucleic acid or nucleic acid sequence. Homology is generally inferred from sequence similarity between two or more nucleic acids or proteins (or sequences thereof). The precise percentage of similarity between sequences that is useful in establishing homology varies with the nucleic acid and protein at issue, but as little as 25% sequence similarity over about 50, about 100, about 150 or more residues is routinely used to establish homology. Higher levels of sequence similarity, e.g., at least about 30%, about 40%, about 50%, about 60%, about 70%, about 80%, about 90%, about 95%, or about 99% or more, can also be used to establish homology. Higher levels of sequence similarity, e.g., at least 30%, 40%, 50%, 60%, 70%, 80%, 90%, 95%, or 99% or more, can also be used to establish homology

Methods for determining sequence similarity percentages (e.g., BLAST protein (BLASTP) and nucleotide (BLASTN) using default parameters) are described herein and are generally available. For sequence comparison and homology determination, typically one sequence acts as a reference sequence to which test sequences are compared. When using a sequence comparison algorithm, test and reference sequences can be input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters. Optimal alignment of sequences for comparison are known to those skilled in the art.

In some embodiments, the engineered nucleic acid processing enzyme of the present disclosure comprises a mutation. In one embodiment, the mutation is a substitution in the polymerase domain of the engineered nucleic acid processing enzyme. In some embodiments, the polymerase domain comprises a substitution at positions 141 and 143 of SEQ ID NO: 1 or 2. In some embodiment, the polymerase domain comprises a substitution at position 141 of SEQ ID NO: 2.

In one embodiment, the engineered nucleic acid processing enzyme lacks proofreading activity (3′-5′ exonuclease). Methods for inactivating the exonuclease activity of an enzyme via genetic engineered disruption of the exonuclease domain are well known in the art. In some embodiments, the exonuclease deficient enzyme comprises D141A and E143A in SEQ ID NO: 1 or 2. In some embodiments, the engineered nucleic acid processing enzyme has proofreading activity. In such an embodiment, the disclosed engineered nucleic acid processing enzyme shows at least two, at least three, or least four fold improvement in fidelity over existing reverse transcriptases. As used herein, the “exonuclease domain” refers to the amino acids of the polymerase that binds to the primer terminus in the editing mode for removing misincorporations. This mechanism is important for proofreading (3′-5′ exonuclease) and contributes to processivity.

In some embodiments of the engineered nucleic acid processing enzyme described herein, the polymerase domain comprises: (a) an aspartic acid substitution at position 141; (b) a glutamic acid substitution at position 143; (c) an alanine substitution at position 485; (d) a valine substitution at position 93; (e) an arginine substitution at position 97; (f) a tyrosine substitution at position 384; (g) a valine substitution at position 389; (h) a phenylalanine substitution at position 493; (i) a phenylalanine substitution at position 587; (j) a glutamic acid substitution at position 664; (k) a glycine substitution at position; (l) a tryptophan substitution at position 768; (m) an isoleucine substitution at position 2; (n) an isoleucine substitution at position 38; (o) a lysine substitution at position 118; (p) a methionine substitution at position 137; (q) an arginine substitution at position 381; (r) a lysine substitution at position 466; (s) a tyrosine substitution at position 514; (t) an isoleucine substitution at position 521; and/or (u) an asparagine substitution at position 735 in SEQ ID NO: 1.

In some embodiments, the polymerase domain of the engineered nucleic acid processing enzyme as described herein comprises one or more substitutions selected from the group consisting of an aspartic acid to alanine substitution at position 141 (D141A); a glutamic acid to alanine substitution at position 143 (E143A); an alanine to leucine substitution at position 485 (A485L); a valine to glutamine substitution at position 93 (V93Q); an arginine to methionine substitution at position 97 (R97M); a tyrosine to histidine substitution at position 384 (Y384H); a valine to isoleucine substitution at position 389 (V389I); a phenylalanine to leucine substitution at position 493 (F493L); a phenylalanine to leucine substitution at position 587 (F587L); a glutamic acid to lysine substitution at position 664 (E664K); a glycine to valine substitution at position (G711V); a tryptophan to arginine substitution at position 768 (W768R); an isoleucine to valine substitution at position 2 (I2V); an isoleucine to leucine substitution at position 38 (I38L); a lysine to isoleucine substitution at position 118 (K118I); a methionine to leucine substitution at position 137 (M137L); an arginine to histidine substitution at position 381 (R381H); a lysine to arginine substitution at position 466 (K466R); a tyrosine to isoleucine substitution at position 514 (T514I); an isoleucine to leucine substitution at position 521 (I521L); and an asparagine to lysine substitution at position 735 (N735K) of SEQ ID NO: 1.

In one embodiment, the polymerase domain of the engineered nucleic acid processing enzyme as described herein comprises at least one, at least two, at least three, at least five, at least six, at least seven, at least eight, at least nine, at least ten, at least eleven, at least twelve, at least fifteen, or at least twenty of the substitutions disclosed herein in SEQ ID NO: 1.

In one embodiment, the polymerase domain of the engineered nucleic acid processing enzyme described herein comprises a combination of R97M, D141A, E143A, Y384H, V389I, Y493L, F587L, E664K, G711V, and W768R substitutions in SEQ ID NO: 1. In another embodiment, the polymerase domain of the engineered nucleic acid processing enzyme described herein comprises I2V, I38L, R97M, K118I, M137L, E143A, R381H; Y384H, V389I, K466R, F493L, T514I, I521L, F587L, E664K, G711V, N735K, and W768R substitutions in SEQ ID NO: 1. Yet in another embodiment, the polymerase domain of the engineered thermostable reverse transcriptase enzyme described herein comprises I2V, I38L, R97M, K118I, M137L, D141A, E143A, R381H, Y384H, V389I, K466R, F493L, T514I, I521L, F587L, E664K, G711V, N735K, and W768R substitutions in SEQ ID NO: 1. Alternatively, the polymerase domain of the engineered nucleic acid processing enzyme described herein comprises I2V, I38L, V93Q, R97M, K118I, M137L, D141A, E143A, R381H, Y384H, A485L, V389I, K466R, F493L, T514I, I521L, F587L, E664K, G711V, N735K, and W768R substitutions in SEQ ID NO: 1.

B. DNA Binding Domains

In some embodiments, the engineered nucleic acid processing enzyme of the present disclosure comprises a nucleic acid binding domain. A DNA binding domain is a protein, or a defined region of a protein, that binds to a nucleic acid in a sequence-independent matter. For example, binding of the protein to DNA does not exhibit any preference for a particular sequence. In some embodiments, the DNA binding domain may be single or double stranded. In one embodiment, the nucleic acid binding domain comprises a single stranded DNA binding protein; a double stranded DNA binding protein; a single stranded RNA binding protein; a double stranded RNA binding protein; a continuous RNA-DNA hybrid binding protein; or a discontinuous RNA-DNA hybrid binding protein.

In some embodiments, the nucleic acid binding domain helps stabilize the interaction between the RNA template and the DNA primer during reverse transcription. In one embodiment, the nucleic acid binding domain enhances the efficiency and/or processivity of the engineered thermostable enzyme during reverse transcription. In some embodiments, a suitable DNA binding domains for the present disclosure is identical to or substantially identical to a known DNA binding protein over a comparison window of about 25 amino acids, about 50- about 100 amino acids, or over the length of the entire protein. The sequence can be compared and aligned for maximum correspondence over a comparison window, or designated region as measured using one of the described comparison algorithms or by manual alignment and visual inspection. For purposes of this patent, percent amino acid identity is determined by the default parameters of BLAST and or CLUSTAL W.

In some embodiments, the nucleic acid binding domain of the engineered nucleic acid processing enzyme comprises a nucleic acid binding protein selected from the group consisting of a histone-like protein, an archaeal basic nucleic acid binding protein, a basic DNA binding domain, HMf-like protein, HU-like protein, HU-family DNA binding protein, Sm-like protein domain, proliferating cell nuclear antigen (PCNA), HU, sto7, Sso7d, Sac7d, and Sac7e. Additional DNA binding domains suitable for use in the engineered nucleic acid processing enzyme of the present disclosure can be identified by homology with known DNA binding proteins and/or by antibody cross reactivity, or may be found by means of a biochemical assay. DNA binding domains may be synthesized or isolated using the techniques described above.

1. Hu Proteins

HU belongs to the family of architectural nuclear proteins that control DNA topology by introducing bends into double stranded (ds) DNA and stabilize higher-order nucleoprotein. HU is a histone like DNA binding protein that was initially identified and characterized in Escherichia coli strain U93. HU is known for its DNA bending activity and binds dsDNA in a nonspecific manner and with low affinity. However, HU has also been shown to bind to distorted DNA with a higher binding affinity.

HU is a particularly important DNA binding protein for engineering the recombinant thermostable reverse transcriptase enzyme of the present disclosure because HU is the only bacterial protein that clearly exhibits a dual specific DNA and RNA binding activity. HU binds to double-stranded DNA, double-stranded RNA, and linear DNA-RNA duplexes with a similar low affinity. In addition, HU is able to bind all forms of nucleic acids, including DNA-RNA hybrids. HU binding to discontinuous DNA-RNA structures is much stronger than that to DNA and RNA duplexes. HU also displays the same affinity with nicked DNA, which is one of the structures that HU binds specifically. Thus, HU actually has shown higher affinity for DNA-RNA hybrids than for RNA or DNA duplexes. See Balandina, J. Biol. Chem., 277, 27622-27628 (2002); Stojkova et al., Front. Cell. Infect. Microbiol., 9:159 (2019). Because of its ability to bind all forms of nucleic acids, HU nucleic acid binding domain and its fusion to the thermostable reverse transcriptase enzyme disclosed herein provides a novel and non-obvious tool for overcoming the limitations associated with sequencing RNA templates and or using RNA templates in the single cell analysis system disclosed herein, in spatial array and single cell transcriptomics assays, or any RT-PCR reactions as known in the art.

In addition to its nucleic acid-binding ability, HU protein helps stabilize nucleic acid against thermal denaturation. HU protein from thermophilic bacteria, such as Spiroplasma melliferum or Thermotoga maritima, have high thermal stability.

While eukaryotic proteins with dual specific DNA and RNA binding activity have been characterized, most of these proteins possess separate DNA- and RNA-binding domains, and some of them contain multifunctional domains. For example, some of these proteins possess the RNA recognition motifs (RRMs), which is found in RNA and single-strand-binding protein (e.g., at least two transcription factors use an RRM for preferential binding to double-stranded DNA such as the zinc finger domains of TFIIIA, PEP, MOK2, and WT-1 proteins mediate specific binding to both RNA and dsDNA). The RRM of murine IPEB protein recognizes both dsDNA and pre-mRNA. The Cys2-His2 zinc fingers represent the canonical DNA-binding motif, which is able to interact specifically with RNA. The TFIIIA transcription factor, contains zinc finger modules specialized for either DNA or RNA recognition.

In contrast, HU does not possess any sequence or structural homology to RRMs or zinc finger motifs. Rather HU has a small DNA-binding domain formed from two β-ribbon arms and an α-helical core, which is able to bind with high specificity to e.g., RpoS mRNA. The simple, straightforward structure of HU binding domain with two highly flexible β-ribbon arms and an α-helical platform is an alternative model for the elaborate binding domains of the eukaryotic proteins that display dual DNA- and RNA-specific binding capacities. The HU protein provides an extension to this bifunctional DNA and RNA binding module. The structure of HU consists solely of the concave, positively charged surface made up of β-ribbon arms and an α-helical hydrophobic core. So far, no other protein has been shown to bind to DNA-RNA hybrids with the same specificity as HU. Furthermore, HU is capable of forming complexes with both RNA 3′- and 5′-overhangs. HU also binds to DNA-RNA 5′-overhangs. This specific interaction is possible because of the intermediate A/B conformation of DNA-RNA hybrid of the double strand part of the DNA-RNA hybrid structure is closer to the A-form RNA helix. As such HU is able to interact not only with the minor groove (as it does for DNA) but also with the narrowed RNA major groove. The ability to bind both minor and major RNA grooves is an unusual feature among nucleic acid-binding proteins. Accordingly, the presence of HU in the engineered nucleic acid processing enzyme disclosed herein is a novel distinguishing feature not found in the art.

In some embodiments, the nucleic acid binding domain of the engineered nucleic acid processing enzyme is a histone-like protein. In some embodiments, the nucleic acid binding domain comprises a bacterial histone-like protein or a bacterial HU-family DNA binding protein. In another embodiment, the nucleic acid binding domain is a Thermus thermophile HU-family DNA binding protein.

In some embodiments, the nucleic acid binding domain binds a DNA-RNA hybrid complex. The DNA-RNA hybrid is continuous or discontinuous. DNA-RNA hybrid is a DNA structure in which one of the DNA stands is replaced with RNA. As used herein, “continuous DNA-RNA hybrid” refers to a DNA-RNA hybrid that does not contain a single strand break, which can be incorporated with a nick (e.g., nick DNA), or DNA-RNA hybrid that does not contain a DNA or RNA 3′- and 5′-overhangs. As used herein, “discontinuous DNA-RNA hybrid” refers to a DNA-RNA hybrid containing a single strand break, which can be incorporated with a nick (e.g., nick DNA), or DNA-RNA hybrid containing a DNA or RNA 3′- and 5′-overhangs. These terms have the same meaning as those used in the art. Those of skilled in the art understand that nick and 3′-overhang structures are DNA replication intermediates. Indeed, during DNA replication, the overall growth of the antiparallel two daughter DNA chains appears to occur 5′-to-3′ direction in the leading-strand and 3′-to-5′ direction in the lagging-strand using enzyme system only able to elongate 5′-to-3′ direction. The lagging strand multistep synthesis reactions, called Discontinuous Replication Mechanism, involve short RNA primer synthesis, primer-dependent short DNA chains (Okazaki fragments) synthesis, primer removal from the Okazaki fragments and gap filling between Okazaki fragments by RNase H and DNA polymerase I, and long lagging strand formation by joining between Okazaki fragments with DNA ligase. See e.g., Okazaki T, Proc Jpn Acad Ser B Phys Biol Sci. 93(5): 322-338 (2017).

Accordingly, HU's ability to bind DNA-RNA hybrid complements and enhances the efficiency and processive characteristics of the engineered nucleic acid processing enzyme of the present disclosure. Indeed endogenous polymerases possess at least three properties: (1) the 5′-to-3′ polymerase activity, (2) the 5′-to-3′ exonuclease activity, which is specific to double strand DNA or RNA-DNA hybrid molecules, and (3) the 3′-to-5′ exonuclease activity, which is specific to single-stranded DNA substrate and provides the proofreading function. When the 5′-to-3′ polymerase and the 5′-to-3′ exonuclease activities function in a coordinated manner, a nick on the double strand DNA migrates towards the 3′ direction and is eventually filled.

In some embodiments, HU recognizes both nicked and 3′-overhang structures in which one of the DNA strands is replaced with RNA. Specifically, the DNA-RNA 3′-overhang is involved in the priming repair of the double-stranded breaks. Indeed, HU binds nicked and 3′-overhang structures in RNA with apparent dissociation constants (Kd) of 10 and 16 nM for nicked RNA and 3′-overhang RNA, respectively. Conversely, HU binds nicked and 3′-overhang structures in DNA with identical apparent dissociation constants (Kd) to the values found for nicked RNA and RNA 3′-overhang. Furthermore, the apparent dissociation constants (Kd) of HU for discontinuous DNA-RNA hybrid is 100× stronger than that for double-stranded nucleic acids.

In some embodiments, the nucleic acid binding protein of the engineered nucleic acid processing enzyme comprises an amino acid sequence having at least 90%, at least 91%, at least 92%, at least 93%, at least 94%, at least 95%, at least 96%, at least 97%, at least 98%, or at least 99% sequence identity to the amino acid sequence of SEQ ID NO: 6. In some embodiments, the nucleic acid binding protein of the engineered nucleic acid processing enzyme comprises an amino acid sequence having 90%, 91%, 92%, 93%, 94%, 95%, 96%, 97%, 98%, or 99% sequence identity to the amino acid sequence of SEQ ID NO: 6. In some embodiments, the nucleic acid binding protein of the engineered nucleic acid processing enzyme comprises an amino acid sequence set forth in SEQ ID NO: 6 or an amino acid sequence having at least 90% sequence identity to the amino acid sequence set forth in SEQ ID NO: 6.

In some embodiments, the engineered nucleic acid processing enzyme of the present disclosure comprises an amino acid sequence of SEQ ID NO: 5; or an amino acid sequence having at least about 90% sequence identity to the amino acid sequence set forth in SEQ ID NO: 5. In some embodiments, the engineered thermostable reverse transcriptase enzyme comprises an amino acid sequence of SEQ ID NO: 4; or an amino acid sequence having at least 90% sequence identity to the amino acid sequence of SEQ ID NO: 4.

In some embodiments, the thermostable reverse transcriptase enzyme has a dual DNA and RNA polymerase activity, or an RNA reverse transcriptase activity.

2. PCNA

Many but not all family B DNA polymerases interact with accessory proteins to achieve highly processive DNA synthesis. A particularly important class of accessory proteins is referred to as the sliding clamps. Several characterized sliding clamps exist as trimers in solution, and can form a ring-like structure with a central passage capable of accommodating double-stranded DNA. The sliding clamp forms specific interactions with the amino acids located at the C terminus of particular DNA polymerases, and tethers those polymerases to the DNA template during replication. The sliding clamp in eukarya is referred to as the proliferating cell nuclear antigen (PCNA), while similar proteins in other organisms are often referred to as PCNA homologs. These homologs have marked structural similarity but limited sequence similarity.

Recently, PCNA homologs have been identified from thermophilic archaea (e.g., Pyroccocusfuriosus). Some family B polymerases in archaea have a C-terminus containing a consensus PCNA-interacting amino acid sequence and are capable of using a PCNA homolog as a processivity factor. See Cann et al., J. Bacteriol., 181:6591-6599 (1999); De Felice et al., J. Mol. Biol., 291:47-57 (1999). These PCNA homologs are useful DNA binding domains for the present disclosure. For example, a consensus PCNA-interacting sequence can be joined to a polymerase that does not naturally interact with a PCNA homolog, thereby allowing a PCNA homolog to serve as a processivity factor for the polymerase. For example, the PCNA-interacting sequence from Pyrococcus furiosus PolII, which is a heterodimeric DNA polymerase containing two family B-like polypeptides, can be covalently joined to Pyrococcus furiosus Poll, a monomeric family B polymerase that does not normally interact with a PCNA homolog. The resulting fusion protein can then be allowed to associate non-covalently with the Pyrococcus furiosus PCNA homolog to generate a novel heterologous protein with increased processivity relative to the unmodified Pyrococcus furiosus Poll.

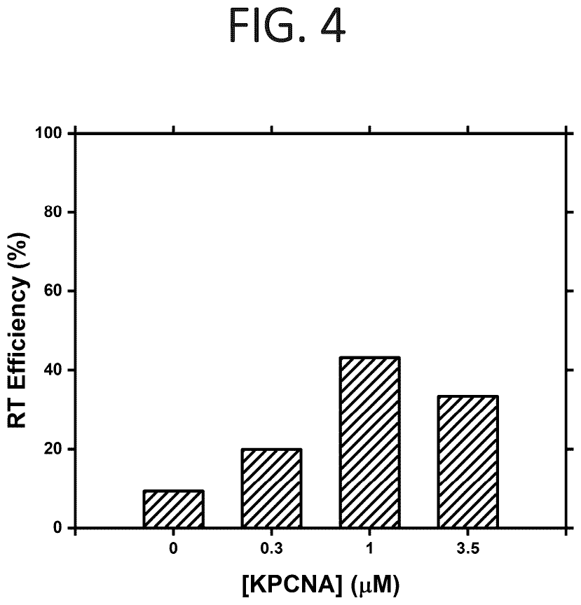

As further shown in FIG. 4, the addition of a T. kodakarensis PCNA (KPCNA) enhanced the reverse transcriptase activity of the engineered thermostable polymerase enzyme described herein (e.g., TgoRT or TgoRTxo) in a dose dependent manner. In the presence of KPCNA, the reverse transcriptase activity of TgoRTxo was enhanced by at least a factor of four. In some embodiments, the nucleic acid binding domain of the engineered nucleic acid processing enzyme of the present disclosure is PCNA. In one embodiment, the PCNA is a T. kodakarensis PCNA. In one embodiment, the PCNA comprises an amino acid sequence of SEQ ID NO: 16. In one embodiment the PCNA comprises a sequence having at least 90% sequence identity to SEQ ID NO: 16.

In some embodiments of the present disclosure, the engineered thermostable reverse transcriptase comprises a first domain comprising a polymerase domain, a second domain conjugated to the first domain that comprises a nucleic acid binding domain; and a third domain comprising a sliding clamp. In this embodiment, the polymerase domain comprises an amino acid sequence of an engineered Thermococcus gorgonarius polymerase (Tgo polymerase). In another embodiment, the sliding clamp is a PCNA. In another embodiment, the sliding clamp is a T. kodakarensis PCNA (KPCNA). In one embodiment, the engineered thermostable reverse transcriptase comprises a polynucleotide encoding the recombinant Tgo polymerase of the present disclosure; a polynucleotide encoding a HU polypeptide; and a polynucleotide encoding a PCNA polypeptide. In some embodiments, the engineered thermostable reverse transcriptase comprises a polynucleotide encoding the amino acid sequence of SEQ ID NO: 1 or an amino acid sequence having at least 90% sequence identity to the amino acid sequence of SEQ ID NO: 1; a polynucleotide encoding the amino acid sequence of SEQ ID NO: 6 or a sequence having at least 90% sequence identity to the amino acid sequence of SEQ ID NO: 6; and a polynucleotide encoding the amino acid sequence of SEQ ID NO: 16 or a sequence having at least 90% sequence identity to the amino acid sequence of SEQ ID NO: 16.

3. Sso7d and Sso7d-Like Proteins

In some embodiments, the nucleic acid binding domain of the engineered nucleic acid processing enzyme is a Sso7d, a Sso7d like, or a Sso7d nucleic acid binding domain. Sso7d and Sso7d-like proteins, Sac7d and Sac7d-like proteins (e.g., Sac7a, Sac7b, Sac7d, and Sac7e) are small (about 7,000 Kd MW), basic chromosomal proteins from the hyperthermophilic archaebacteria Sulfolobus solfataricus and S. acidocaldarius, respectively. These proteins are lysine-rich and have high thermal, acid and chemical stability. They bind DNA in a sequence-independent manner and when bound, increase the melting temperature (TM) of DNA by up to 40° C. See McAfee et al., Biochemistry, 34:10063-10077 (1995). These proteins and their homologs are typically believed to be involved in stabilizing genomic DNA at elevated temperatures. Suitable Sso7d-like DNA binding domains for use in the invention can be modified based on their sequence homology to Sso7d.

4. HMf-Like Proteins

Certain helix-hairpin-helix motifs have been shown to bind DNA nonspecifically and enhance the processivity of a DNA polymerase to which it is fused. See Pavlov et al., Proc Natl Acad Sci USA, 99:13510-5 (2002). In some embodiments, the nucleic acid binding domain of the engineered nucleic acid processing enzyme is a Hmf-like protein. Hmf-like proteins are archaeal histones that share homology both in amino acid sequences and in structure with eukaryotic H4 histones, which are thought to interact directly with DNA. The HMf family of proteins form stable dimers in solution, and several HMf homologs have been identified from thermostable species (e.g., Methanothermus fervidus and Pyrococcus strain GB-3a). Once joined to a polymerase or any nucleic acid (e.g., DNA) modifying enzyme with a low intrinsic processivity, the HMf family of proteins can enhance the ability of the enzyme to slide along the DNA substrate and thus increase its processivity. For example, the dimeric HMf-like protein can be covalently linked to the N terminus of DNA polymerase. In some embodiments, the covalent linkage is via chemical modification, thereby improving the processivity of the polymerase.

5. Engineering a Recombinant Thermostable Reverse Transcriptase

The DNA binding domain and the polymerase domain of the engineered thermostable reverse transcriptase or the reverse transcriptase fusion proteins of the present disclosure can be joined by methods well known to those of skill in the art. These methods include both chemical and recombinant means.

In some embodiments, the coding sequences of each polypeptide in a resulting fusion protein are directly joined at their amino- or carboxy-terminus via a peptide bond in any order. Alternatively, an amino acid linker sequence may be employed to separate the first and second polypeptide components by a distance sufficient to ensure that each polypeptide folds into its secondary and tertiary structures. Such an amino acid linker sequence is incorporated into the fusion protein using standard techniques well known in the art. Suitable peptide linker sequences may be chosen based on the following factors: (1) their ability to adopt a flexible extended conformation; (2) their inability to adopt a secondary structure that could interact with functional epitopes on the first and second polypeptides; and (3) the lack of hydrophobic or charged residues that might react with the polypeptide functional epitopes. Typical peptide linker sequences contain Gly, Ser, Val, and Thr residues. Other near neutral amino acids, such as Ala can also be used in the linker sequence. Amino acid sequences which may be usefully employed as linkers include those disclosed in Maratea et al. (1985) Gene 40:39-46; Murphy et al. Proc. Natl. Acad. Sci. USA, 83:8258-8262 (1986); U.S. Pat. Nos. 4,935,233 and 4,751,180. The linker sequence may generally be from 1 to about 50 amino acids in length, e.g., about 3, about 4, about 6, or about 10 amino acids in length, but can be about 100 or about 200 amino acids in length, or any value in between these numbers (e.g., about 15, about 20, about 101, about 102, about 130 amino acids, etc.). Linker sequences may not be required when the first and second polypeptides have non-essential N-terminal amino acid regions that can be used to separate the functional domains and prevent steric interference.

In some embodiments, the first domain and the second domain of the engineered nucleic acid processing enzyme are linked via a chemical means. Chemical means of joining a DNA binding protein to a polymerase domain are described, e.g., in Bioconjugate Techniques, Hermanson, Ed., Academic Press (1996). These include, for example, derivitization for the purpose of linking the two proteins to each other, either directly or through a linking compound, by methods that are well known in the art of protein chemistry. For example, in one chemical conjugation embodiment, the means of linking the catalytic domain and the DNA binding domain comprises a heterobifunctional-coupling reagent which ultimately contributes to formation of an intermolecular disulfide bond between the two moieties. Other types of coupling reagents that are useful in this capacity for the present invention are described, for example, in U.S. Pat. No. 4,545,985. Alternatively, an intermolecular disulfide may conveniently be formed between cysteines in each moiety, which occur naturally or are inserted by genetic engineering. The means of linking moieties may also use thioether linkages between heterobifunctional crosslinking reagents or specific low pH cleavable crosslinkers or specific protease cleavable linkers or other cleavable or noncleavable chemical linkages.

The means of linking a DNA binding domain, e.g., Hu, and a polymerase domain, may also comprise a peptidyl bond formed between moieties that are separately synthesized by standard peptide synthesis chemistry or recombinant means. The conjugate protein itself can also be produced using chemical methods to synthesize an amino acid sequence in whole or in part. For example, peptides can be synthesized by solid phase techniques, such as, e.g., the Merrifield solid phase synthesis method, in which amino acids are sequentially added to a growing chain of amino acids (see, Merrifield (1963) J. Am. Chem. Soc., 85:2149-2146). Equipment for automated synthesis of polypeptides is commercially available from suppliers such as PE Corp. (Foster City, Calif.), and may generally be operated according to the manufacturer's instructions. The synthesized peptides can then be cleaved from the resin, and purified, e.g., by preparative high performance liquid chromatography (see Creighton, Proteins Structures and Molecular Principles, 50-60 (1983)). The composition of the synthetic polypeptides or of subfragments of the polypeptide, may be confirmed by amino acid analysis or sequencing (e.g., the Edman degradation procedure; see Creighton, Proteins, Structures and Molecular Principles, pp. 34-49 (1983)).

In addition, nonclassical amino acids or chemical amino acid analogs can be introduced as a substitution or addition into the sequence. Non-classical amino acids include, but are not limited to, the D-isomers of the common amino acids, α-amino isobutyric acid, 4-aminobutyric acid, Abu, 2-amino butyric acid, γ-Abu, ε-Ahx, 6-amino hexanoic acid, Aib, 2-amino isobutyric acid, 3-amino propionic acid, ornithine, norleucine, norvaline, hydroxy-proline, sarcosine, citrulline, cysteic acid, t-butylglycine, t-butylalanine, phenylglycine, cyclohexylalanine, β-alanine, fluoro-amino acids, designer amino acids such as β-methyl amino acids, Cα-methyl amino acids, Na-methyl amino acids, and amino acid analogs in general.

C. Tag Proteins

One aspect of the present disclosure provides an engineered nucleic acid processing enzyme comprising: a first domain comprising a polymerase domain; a second domain conjugated to the first domain; and a tag protein. In some embodiments, the polymerase domain comprises an amino acid sequence of an engineered Thermococcus gorgonarius polymerase (Tgo polymerase). In another embodiment, the second domain comprises a nucleic acid binding domain.

In some embodiments, the engineered thermostable reverse transcriptase described herein further comprises a tag protein selected from the group consisting of an affinity tag, a fluorescent tag, or an expression and/or solubility enhancement tag. In some embodiments, the tag protein is selected from hexahistidine tag (his-tag), Fasciola hepatica 8-kDa antigen tag (Fh8), Glutathione-S-transferase (GST) tag, maltose-binding protein tag (MBP), FLAg tag peptide (FLAG tag), streptavidin binding peptide tag (Strep-II), calmodulin-binding protein tag (CBP), mutated dehalogenase tag (HaloTag), staphylococcal Protein A (Protein A), intein mediated purification with the chitin-binding domain (IMPACT (CBD)), cellulose-binding module (CBM), dockerin domain of Clostridium josui tag (Dock), fungal avidin-like protein (Tamavidin), small ubiquitin-like modifier tag (SUMO), a strep tag, Thioredoxin (Trx) tag, aVariFlex C-Terminal solubility enhancement tag, a short peptide C-terminal tag, Solubility-enhancer peptide sequences (SET) tag, IgG domain B1 of Protein G (GB1) tag, IgG repeat domain ZZ of Protein A (ZZ) tag, Mutated dehalogenase tag (HaloTag), Solubility eNhancing Ubiquitous Tag (SNUT tag), Seventeen kilodalton protein (Skp tag), Phage T7 protein kinase (T7PK) tag, E. coli secreted protein A (EspA) tag, Monomeric bacteriophage T7 0.3 protein (Orc protein) (Mocr) tag, E. coli trypsin inhibitor (Ecotin) tag, Calcium-binding protein (CaBP) tag, Stress-responsive arsenate reductase (ArsC) tag, N-terminal fragment of translation initiation factor IF2 (IF2-domain I) tag, N-terminal fragment of translation initiation factor IF2 (Expressivity) tag, Stress-responsive proteins tag (e.g., RpoA, tag, SlyD Tsf tag, RpoS tag, PotD tag, or Crr tag), and E. coli acidic proteins tag (e.g., msyB tag, yigD tag, and rpoD tag). Additional affinity tags and solubility enhancer tags are known to those skill in the art. See Costa et al., Front. Microbiol., 63(5): (2014); Esposito and Chatterjee Curr. Opin. Biotechnol., 17: 353-358 (2006); Malhotra, A. “Tagging for protein expression,” in Guide to Protein Purification, 2nd Edn, eds. R. R. Burgess and M. P. Deutscher (San Diego, CA: Elsevier), 463:239-258 (2009).

In some embodiments, the tag is selected from hexahistidine tag (his-tag), small ubiquitin-like modifier tag (SUMO), aVariFlex C-Terminal solubility enhancement tag, a short peptide C-terminal tag, Thioredoxin (Trx) tag, aVariFlex C-Terminal solubility enhancement tag, Solubility-enhancer peptide sequences (SET) tag, IgG domain B1 of Protein G (GB1) tag, IgG repeat domain ZZ of Protein A (ZZ) tag, Solubility enhancing Ubiquitous Tag (SNUT tag), Seventeen kilodalton protein (Skp tag), Phage T7 protein kinase (T7PK) tag, E. coli secreted protein A (EspA) tag, Monomeric bacteriophage T7 0.3 protein (Orc protein) (Mocr) tag, E. coli trypsin inhibitor (Ecotin) tag, Calcium-binding protein (CaBP) tag, Stress-responsive arsenate reductase (ArsC) tag, N-terminal fragment of translation initiation factor IF2 (IF2-domain I) tag, N-terminal fragment of translation initiation factor IF2 (Expressivity) tag, Fasciola hepatica 8-kDa antigen tag (Fh8), Glutathione-S-transferase (GST) tag, maltose-binding protein tag (MBP), FLAg tag peptide (FLAG), streptavidin binding peptide tag (Strep-II; strep), calmodulin-binding protein tag (CBP), mutated dehalogenase tag (HaloTag), staphylococcal Protein A (Protein A), intein mediated purification with the chitin-binding domain (IMPACT (CBD)), cellulose-binding module (CBM), dockerin domain of Clostridium josui tag (Dock), or fungal avidin-like protein (Tamavidin).

Tags used in the practice of the invention may serve any number of purposes and a number of tags may be added to impart one or more different functions to the engineered reverse transcriptase, and/or derivatives thereof, of the disclosure. For example, tags may (1) contribute to protein-protein interactions both internally within a protein and with other protein molecules, (2) make the protein amenable to particular purification methods, (3) enable one to identify whether the protein is present in a composition; or (4) give the protein other functional characteristics.

In one embodiment, the tag is an affinity tag selected from a histidine tag such as a hexahistidine tag (his-tag or 6 His-tag), Fasciola hepatica 8-kDa antigen tag (Fh8), Glutathione-S-transferase (GST) tag, maltose-binding protein tag (MBP), FLAg tag peptide (FLAG), streptavidin binding peptide tag (Strep-II), calmodulin-binding protein tag (CBP), mutated dehalogenase tag (HaloTag), staphylococcal Protein A (Protein A), intein mediated purification with the chitin-binding domain (IMPACT (CBD)), cellulose-binding module (CBM), dockerin domain of Clostridium josui tag (Dock), fungal avidin-like protein (Tamavidin). In one embodiment, the tag is a hexahistidine tag.

In some embodiments, the tag is selected from a small ubiquitin-like modifier tag (SUMO), a VariFlex C-Terminal solubility enhancement tag, a short peptide C-terminal tag, Thioredoxin (Trx) tag, Solubility-enhancer peptide sequences (SET) tag, IgG domain B1 of Protein G (GB1) tag, IgG repeat domain ZZ of Protein A (ZZ) tag, Solubility enhancing Ubiquitous Tag (SNUT tag), Seventeen kilodalton protein (Skp tag), Phage T7 protein kinase (T7PK) tag, E. coli secreted protein A (EspA) tag, Monomeric bacteriophage T7 0.3 protein (Orc protein) (Mocr) tag, E. coli trypsin inhibitor (Ecotin) tag, Calcium-binding protein (CaBP) tag, Stress-responsive arsenate reductase (ArsC) tag, N-terminal fragment of translation initiation factor IF2 (IF2-domain I) tag, N-terminal fragment of translation initiation factor IF2 (Expressivity) tag, Fasciola hepatica 8-kDa antigen tag (Fh8), Glutathione-S-transferase (GST) tag, maltose-binding protein tag (MBP), FLAg tag peptide (FLAG), streptavidin binding peptide tag (Strep-II; strep), calmodulin-binding protein tag (CBP), mutated dehalogenase tag (HaloTag), staphylococcal Protein A (Protein A), intein mediated purification with the chitin-binding domain (IMPACT (CBD)), cellulose-binding module (CBM), dockerin domain of Clostridium josui tag (Dock), fungal avidin-like protein (Tamavidin).

In some embodiments, the solubility enhancer tag is selected from the group consisting of a SUMO tag, a GST tag, a Trx tag, aVariFlex C-Terminal solubility enhancement tag, a short peptide C-terminal tag, an Fh8 tag, MBP tag, SET tag, GB1 tag, ZZ tag, HaloTag, SNUT tag, Skp tag, T7PK tag, EspA tag, Mocr tag, Ecotin tag, CaBO tag, ArsC tag, IF2-domain I tag, Expressivity tag, RpoA, tag, SlyD, tag, Tsf tag, RpoS tag, PotD tag, Crr tag, msyB tag, yigD tag, and rpoD tag.

In some embodiments, the tag is an affinity tag. In one embodiment, the tag is an affinity tag and comprises a histidine purification tag. In one embodiment, the tag is a hexahistidine tag (his tag). In one embodiment, the tag comprises an amino acid sequence of the sequence HHFIHIH (SEQ ID NO: 9). In one embodiment, the tag is a solubility enhancer tag. In one embodiment, the solubility enhancer tag is a short peptide C-terminal tag. In one embodiment, the solubility enhancer tag comprises an amino acid sequence of SEEDEEKEEDG (SEQ ID NO: 10) or an amino acid sequence having at least 90% sequence identity to the amino acid sequence of SEQ ID NO: 10.

In some embodiments, the tag further comprises an endoprotein cleavage site selected from ENLYFQ/G (SEQ ID NO: 11), DDDDK/(SEQ ID NO: 12), IEGR/(SEQ ID NO: 13), LVPR/GS (SEQ ID NO: 14), or LEVLFQ/GP (SEQ ID NO: 15).

In some embodiments, the engineered nucleic acid processing enzyme or a derivative thereof further comprises a protease cleavage sequence. In some embodiments, the cleavage of the protease cleavage sequence by a protease results in cleavage of the affinity tag from the engineered reverse transcriptase enzyme or a derivative thereof. In some instances, the protease cleavage sequence/site is recognized by a protease including, but not limited to, alanine carboxypeptidase, Armillaria mellea astacin, bacterial leucyl aminopeptidase, cancer procoagulant, cathepsin B, clostripain, cytosol alanyl aminopeptidase, elastase, endoproteinase Arg-C, enterokinase (EnTK), gastricsin, gelatinase, Gly-X carboxypeptidase, glycyl endopeptidase, human rhinovirus 3C protease, hypodermin C, Iga-specific serine endopeptidase, leucyl aminopeptidase, leucyl endopeptidase, lysC, lysosomal pro-X carboxypeptidase, lysyl aminopeptidase, methionyl aminopeptidase, myxobacter, nardilysin, pancreatic endopeptidase E, picornain 2A, picornain 3C, proendopeptidase, prolyl aminopeptidase, proprotein convertase I, proprotein convertase II, russellysin, saccharopepsin, semenogelase, T-plasminogen activator, thrombin (Thr), tissue kallikrein, tobacco etch virus (TEV), togavirin, tryptophanyl aminopeptidase, U-plasminogen activator, V8, venombin A, venombin AB, factor Xa (Xa), and Xaa-pro aminopeptidase. In some embodiments, the protease cleavage sequence is a thrombin cleavage sequence.

In some embodiment, the tag is cleaved or removed from the engineered nucleic acid processing enzyme or derivatives thereof via the cleavage site. In one embodiment, the tag is cleaved or removed using an endoprotein selected from the group consisting of tobacco etch virus protease (Tev), enterokinase (EntK), factor Xa (Xa), thrombin (Thr), genetically engineered derivative of human rhinovirus 3C protease (PreScission), Catalytic core of Ulp1 (SUMO protease). In one embodiment, the tag is cleaved at ENLYFQ/G (SEQ ID NO: 11) using tobacco etch virus protease (Tev). In another embodiment, the tag is cleaved at DDDDK/(SEQ ID NO: 12) using Enterokinase (EntK). In another embodiment, the tag is cleaved at IEGR/(SEQ ID NO: 13) using Factor Xa (Xa). In another embodiment, the tag is cleaved at LVPR/GS (SEQ ID NO: 14) using thrombin (Thr). In another embodiment, the tag is cleaved at LEVLFQ/GP (SEQ ID NO: 15) using a genetically engineered derivative of human rhinovirus 3C protease. In another embodiment, the tag is cleaved with Catalytic core of Ulp1 (SUMO protease). Catalytic core of Ulp1 recognizes SUMO tertiary structure and cleaves at the C-terminal end of the conserved Gly-Gly sequence in SUMO.

In some embodiments, the engineered thermostable transcriptase enzyme or derivatives thereof comprises an affinity tag at the N-terminus or at the C-terminus of the amino acid sequence. In some embodiments, the affinity tag include, but is not limited to, albumin binding protein (ABP), AU1 epitope, AU5 epitope, T7-tag, V5-tag, B-tag, Chloramphenicol Acetyl Transferase (CAT), Dihydrofolate reductase (DHFR), AviTag, Calmodulin-tag, polyglutamate tag, E-tag, FLAG-tag, HA-tag, Myc-tag, NE-tag, S-tag, SBP-tag, Doftag 1, Softag 3, Spot-tag, tetracysteine (TC) tag, Ty tag, VSV-tag, Xpress tag, biotin carboxyl carrier protein (BCCP), green fluorescent protein tag, HaloTag, Nus-tag, thioredoxin-tag, Fc-tag, cellulose binding domain, chitin binding protein (CBP), choline-binding domain, galactose binding domain, maltose binding protein (MBP), Horseradish Peroxidase (HRP), Strep-tag, HSV epitope, Ketosteroid isomerase (KSI), KT3 epitope, LacZ, Luciferase, PDZ domain, PDZ ligand, Polyarginine (Arg-tag), Polyaspartate (Asp-tag), Polycysteine (Cys-tag), Polyphenylalanine (Phe-tag), Profinity eXact, Protein C, S1-tag, S1-tag, Staphylococcal protein A (Protein A), Staphylococcal protein G (Protein G), Small Ubiquitin-like Modifier (SUMO), Tandem Affinity Purification (TAP), TrpE, Ubiquitin, Universal, glutathione-S-transferase (GST), and poly(His) tag. In some instances, the affinity tag is at least 5 histidine amino acids.

In some embodiments, the engineered nucleic acid processing enzyme comprises an amino acid sequence of SEQ ID NO: 2; or an amino acid sequence having at least 90% sequence identity to the amino acid sequence of SEQ ID NO: 2. In one embodiment, the engineered nucleic acid processing enzyme comprises an amino acid sequence of SEQ ID NO: 3; or an amino acid sequence having at least 90% sequence identity to the amino acid sequence of SEQ ID NO: 3. In one embodiment, the engineered nucleic acid processing enzyme comprises an amino acid sequence of SEQ ID NO: 4; or an amino acid sequence having at least 90% sequence identity to the amino acid sequence of SEQ ID NO: 4. In one embodiment, the engineered nucleic acid processing enzyme comprises an amino acid sequence of SEQ ID NO: 5; or an amino acid sequence having at least 90% sequence identity to the amino acid sequence of SEQ ID NO: 5. In some embodiments, the engineered nucleic acid processing enzyme or a derivative thereof comprises an amino acid sequence of ENLYFQ/G (SEQ ID NO: 11), DDDDK/(SEQ ID NO: 12), IEGR/(SEQ ID NO: 13), LVPR/GS (SEQ ID NO: 14), or LEVLFQ/GP (SEQ ID NO: 15).

One of skill will recognize that modifications can additionally be made to the polymerases of the present invention without diminishing their biological activity. Some modifications may be made to facilitate the cloning, expression, or incorporation of a domain into a fusion protein. Such modifications are well known to those of skill in the art and include, for example, the addition of codons at either terminus of the polynucleotide that encodes the binding domain to provide, for example, a methionine added at the amino terminus to provide an initiation site, or additional amino acids placed on either terminus to create conveniently located restriction sites or termination codons or purification sequences.

One or more of the domains may also be modified to facilitate the linkage of a variant polymerase domain and DNA binding domain to obtain the polynucleotides that encode the fusion polymerases of the invention. Thus, DNA binding domains and polymerase domains that are modified by such methods are also part of the invention. For example, a codon for a cysteine residue can be placed at either end of a domain so that the domain can be linked by, for example, a sulfide linkage. The modification can be performed using either recombinant or chemical methods (see e.g., Pierce Chemical Co. catalog, Rockford IL).

D. Thermostability and Processivity

1. Thermostability

As used herein, the term “Thermostable” generally refers to an enzyme, such as a reverse transcriptase (“thermostable reverse transcriptase”), which retains a greater percentage or amount of its activity after a heat treatment than is retained by the same enzyme having wild type thermostability or a control enzyme having a certain thermostability, after an identical treatment. Thus, a reverse transcriptase having increased/enhanced thermostability may be defined as a reverse transcriptase having any increase in thermostability, preferably from about 1.2 to about 10,000 fold, from about 1.5 to about 10,000 fold, from about 2 to about 5,000 fold, or from about 2 to about 2000 fold, or any value in between these amounts, and retention of activity after a heat treatment sufficient to cause a reduction in the activity of a reverse transcriptase that is wild type for thermostability or a control enzyme having a certain thermostability. In other aspects of the disclosure, the increase in thermostability can be about 5 fold, about 10 fold, about 25 fold about 50 fold, about 75 fold, about 100 fold, about 150 fold, about 200 fold, about 300 fold, about 400 fold, about 500 fold, about 600 fold, about 700 fold, about 800, about 900 fold, or about 1000 fold.

In other aspects, the increase in thermostability is 1-5 fold, 5-10 fold, 10-15 fold, 15-20 fold, 20-25 fold, 25-30 fold, 30-35 fold, 35-40 fold, 40-45 fold, 45-50 fold, 50-55 fold, 55-60 fold, 60-65 fold, 65-70 fold, 70-75 fold, 75-80 fold, 80-85 fold, 85-90 fold, 90-95 fold, 95-100 fold, 100-105 fold, 105-110 fold, 110-115 fold, 115-120 fold, 120-125 fold, 125-130 fold, 135-135 fold, 135-140 fold, 140-145 fold, 145-150 fold, 150-200 fold, 200-250 fold, 250-300 fold, 300-350 fold, 350-400 fold, 400-450 fold, 450-500 fold, 500-550 fold, 550-600 fold, 600-650 fold, 650-700 fold, 700-750 fold, 750-800 fold, 800-850 fold, 850-900 fold, 900-950 fold or 950-1000 fold.

In other aspects, the increase in thermostability is 10 fold, 11 fold, 12 fold, 13 fold, 14 fold, 15 fold, 16 fold, 17 fold, 18 fold, 19 fold, 20 fold, 21 fold, 22 fold, 23 fold, 24 fold, 25 fold, 26 fold, 27 fold, 28 fold, 29 fold, 30 fold, 31 fold, 32 fold, 33 fold, 34 fold, 35 fold, 36 fold, 37 fold, 38 fold, 39 fold, 40 fold, 42 fold, 44 fold, 46 fold, 48 fold, 50 fold, 52 fold, 54 fold, 56 fold, 58 fold, 60 fold, 62 fold, 64 fold, 68 fold, 70 fold, 72 fold, 74 fold, 76 fold, 78 fold, 80 fold, 82 fold, 84 fold, 86 fold, 88 fold, 90 fold, 92 fold, 94 fold, 96 fold, 98 fold, or 100 fold.

In other aspects, the increase in thermostability is 1.1 fold, 1.2 fold, 1.3 fold, 1.4 fold, 1.5 fold, 1.6 fold, 1.7 fold, 1.8 fold, 1.9 fold, 2.0 fold, 2.1 fold, 2.2 fold, 2.3 fold, 2.4 fold, 2.5 fold, 2.6 fold, 2.7 fold, 2.8 fold, 2.9 fold, 3.0 fold, 3.1 fold, 3.2 fold, 3.3 fold, 3.4 fold, 3.5 fold, 3.6 fold, 3.7 fold, 3.8 fold, 3.9 fold, 4.0 fold, 4.2 fold, 4.4 fold, 4.6 fold, 4.8 fold, 5.0 fold, 5.2 fold, 5.4 fold, 5.6 fold, 5.8 fold, 6.0 fold, 6.2 fold, 6.4 fold, 6.8 fold, 7.0 fold, 7.2 fold, 7.4 fold, 7.6 fold, 7.8 fold, 8.0 fold, 8.2 fold, 8.4 fold, 8.6 fold, 8.8 fold, 9.0 fold, 9.2 fold, 9.4 fold, 9.6 fold, 9.8 fold, or 10.0 fold.

To determine the thermostability of the engineered thermostable reverse transcriptase of the present disclosure, the engineered thermostable reverse transcriptase can be compared to the corresponding wild type Tgo polymerase and/or wild type MMLV or a variant thereof to determine the relative enhancement or increase in thermostability. In a non-limiting example, after a heat treatment at 60° C. for 5 minutes, the engineered thermostable reverse transcriptase may retain approximately 90% of the activity present before the heat treatment, whereas wild type MMLV or MMLV variant may retain 10% of its original activity. Likewise, after a heat treatment at 60° C. for 15 minutes, the engineered thermostable reverse transcriptase may retain approximately 80% of its original activity, whereas wild type MMLV or MMLV variant may have no measurable activity. Similarly, after a heat treatment at 60° C. for 15 minutes, the engineered thermostable reverse transcriptase may retain approximately 50%, approximately 55%, approximately 60%, approximately 65%, approximately 70%, approximately 75%, approximately 80%, approximately 85%, approximately 90%, or approximately 95% of its original activity, whereas wild type MMLV or MMLV variant may have no measurable activity or may retain 20%, 15%, 10%, or none of its original activity. In the first instance (i.e., after heat treatment at 60° C. for 5 minutes), the thermostable reverse transcriptase would be said to be 9-fold more thermostable than the wild type reverse transcriptase (90% compared to 10%). Examples of conditions which may be used to measure thermostability of an enzyme such as reverse transcriptases are set out in further detail below and in the Examples.

The thermostability of a reverse transcriptase (e.g., recombinant thermostable polymerase, recombinant thermophilic polymerase, or engineered thermostable reverse transcriptase as described herein) can be determined, for example, by comparing the residual activity of a reverse transcriptase that has been subjected to a heat treatment, e.g., incubated at a certain temperature, e.g. without limitation 60° C. for a given period of time, for example, five minutes, to a control sample of the same reverse transcriptase that has been incubated at room temperature for the same length of time as the heat treatment. One way the residual activity may be measured is by following the incorporation of a radiolabeled deoxyribonucleotide into an oligodeoxyribonucleotide primer using a complementary oligoribonucleotide template. For example, the ability of the reverse transcriptase to incorporate [α-³²P]-dGTP into an oligo-dG primer using a poly(riboC) template may be assayed to determine the residual activity of the reverse transcriptase. Methods for measuring residual activity of reverse transcriptase and polymerases are known by those of skill in the art. See e.g., Nikiforov, T. T., Anal Biochem., 2011, 412(2): 229-36, which is hereby incorporated by reference.

In some embodiments, the engineered nucleic acid processing enzyme of the present disclosure is thermophilic. In one embodiment, the engineered thermostable reverse transcriptase is resistant to thermal inactivation when compared to a wild-type polymerase. In another embodiment, the engineered thermostable reverse transcriptase is resistant to thermal inactivation at a temperature from about 53° C. to about 75° C.; from about 55° C. to about 75° C.; from about 60° C. to about 75° C.; from about 53° C. to about 68° C.; from about 55° C. to about 68° C.; from about 45° C. to about 68° C.; or from about 50° C. to about 68° C. In yet another embodiment, the engineered thermostable reverse transcriptase is resistant to thermal inactivation at a temperature of about 68° C. In certain embodiments, the engineered enzymes of the invention have high thermostability, e.g., thermostability at temperatures above 50° C., 51° C., 52° C., 53° C., 54° C., 55° C., 56° C., 57° C., 58° C., 59° C., 60° C., 61° C., 62° C., 63° C., 64° C., 65° C., 66° C., 67° C., 68° C., 69° C., 70° C., 71° C., 72° C., 73° C. or more, and optionally have proofreading activity. In certain embodiments, the engineered enzymes of the invention have high thermostability, e.g., thermostability at temperatures of 50° C., 51° C., 52° C., 53° C., 54° C., 55° C., 56° C., 57° C., 58° C., 59° C., 60° C., 61° C., 62° C., 63° C., 64° C., 65° C., 66° C., 67° C., 68° C., 69° C., 70° C., 71° C., 72° C., 73° C. or more, and optionally have proofreading activity. In certain embodiments, the engineered enzymes of the invention have high thermostability, e.g., thermostability at temperatures of about: 50° C., 51° C., 52° C., 53° C., 54° C., 55° C., 56° C., 57° C., 58° C., 59° C., 60° C., 61° C., 62° C., 63° C., 64° C., 65° C., 66° C., 67° C., 68° C., 69° C., 70° C., 71° C., 72° C., 73° C. or more, and optionally have proofreading activity.

In another embodiment, the thermostability of the engineered nucleic acid processing enzyme is determined by measuring the half-life of the engineered nucleic acid processing enzyme. Such half-life may be compared to a control or wild type polymerase enzyme to determine the difference (or delta) in half-life.

2. Half-Life

In some embodiments, the engineered nucleic acid processing enzyme possesses an enhanced half-life when compared to a wild-type polymerase and/or a wild-type reverse transcriptase at a temperature from about 53° C. to about 75° C.; from about 55° C. to about 75° C.; from about 60° C. to about 75° C.; from about 53° C. to about 68° C.; from about 55° C. to about 68° C.; from about 45° C. to about 68° C.; or from about 50° C. to about 68° C. In certain embodiments, half-life of the engineered enzymes of the invention is measured at temperatures above 50° C., 51° C., 52° C., 53° C., 54° C., 55° C., 56° C., 57° C., 58° C., 59° C., 60° C., 61° C., 62° C., 63° C., 64° C., 65° C., 66° C., 67° C., 68° C., 69° C., 70° C., 71° C., 72° C., 73° C. or more, and optionally have proofreading activity. In certain embodiments, half-life of the engineered enzymes of the invention is measured at temperatures of 50° C., 51° C., 52° C., 53° C., 54° C., 55° C., 56° C., 57° C., 58° C., 59° C., 60° C., 61° C., 62° C., 63° C., 64° C., 65° C., 66° C., 67° C., 68° C., 69° C., 70° C., 71° C., 72° C., 73° C. or more, and optionally have proofreading activity. In certain embodiments, half-life of the engineered enzymes of the invention is measured at temperatures of about: 50° C., 51° C., 52° C., 53° C., 54° C., 55° C., 56° C., 57° C., 58° C., 59° C., 60° C., 61° C., 62° C., 63° C., 64° C., 65° C., 66° C., 67° C., 68° C., 69° C., 70° C., 71° C., 72° C., 73° C. or more, and optionally have proofreading activity.

The half-life of the engineered nucleic acid processing enzyme of the disclosure is preferably determined at elevated temperatures (e.g., greater than 37° C.) and preferably at temperatures ranging from 40° C. to 80° C., or temperatures ranging from 45° C. to 750 C, 50° C. to 700 C, 55° C. to 65° C., and 58° C. to 62° C. Preferred half-lives of the engineered nucleic acid processing enzyme of the present disclosure may range from about 4 minutes to about 10 hours, about 4 minutes to about 7.5 hours, about 4 minutes to about 5 hours, about 4 minutes to about 2.5 hours, or about 4 minutes to about 2 hours, depending upon the temperature used. For example, the reverse transcriptase activity of the engineered thermostable reverse transcriptase of the present disclosure may have a half-life of at least about 4 minutes, at least about 5 minutes, at least about 6 minutes, at least about 7 minutes, at least about 8 minutes, at least about 9 minutes, at least about 10 minutes, at least about 11 minutes, at least about 12 minutes, at least about 13 minutes, at least about 14 minutes, at least about 15 minutes, at least about 20 minute, at least about 25 minutes, at least about 30 minutes, at least about 40 minutes, at least about 50 minutes, at least about 60 minutes, at least about 70 minutes, at least about 80 minutes, at least about 90 minutes, at least about 100 minutes, at least about 115 minutes, at least about 125 minutes, at least about 150 minutes, at least about 175 minutes, at least about 200 minutes, at least about 225 minutes, at least about 250 minutes, at least about 275 minutes, at least about 300 minutes, at least about 400 minutes, at least about 500 minutes, or any time period in between these values, at temperatures of about 48° C., about 50° C., about 52° C., about 54° C., about 56° C., about 58° C., about 60° C., about 62° C., about 64° C., about 66° C., about 68° C., and/or about 70° C. In some embodiments, the thermostability of the engineered nucleic acid processing enzyme enhances the half-life of the engineered nucleic acid processing enzyme.

In some embodiments, the engineered nucleic acid processing enzyme possesses one or more of the following characteristics when compared to a wild-type polymerase and/or reverse transcriptase: increased thermostability; increased thermoreactivity; increased resistance to reverse transcriptase inhibitors; increased ability to reverse transcribe difficult templates; increased speed; increased processivity; increased specificity; enhanced polymerization activity; increased sensitivity, or any combination thereof.

3. Processivity

Processivity is defined as the ability of a polymerase to carry out continuous nucleic acid synthesis on a template nucleic acid without frequent dissociation. It can be measured by the average number of nucleotides incorporated by a polymerase on a single association/disassociation event. DNA polymerase alone produces short DNA product strand per binding event. Most DNA polymerases are intrinsically low-processivity enzymes. The low processivity of DNA polymerase alone is insufficient for the timely replication of a large genome.

In some embodiments, the polymerization activity of the engineered nucleic acid processing enzyme as described herein is enhanced by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 90%, or about 100% as compared to the wild-type polymerase.

In some embodiments, the engineered nucleic acid processing enzyme reverse transcribes a RNA molecule having at least about 100, at least about 200, at least about 300, at least about 400, at least about 500, at least about 600, at least about 700, at least about 800, at least about 900, or at least about 1000 nucleotides. In another embodiment, the engineered nucleic acid processing enzyme reverse transcribes a RNA molecule that is at least about 1 kb, at least about 2 kb, at least about 3 kb, at least about 4 kb, at least about 5 kb, at least about 6 kb, at least about 7 kb, at least about 8 kb, at least about 9 kb, at least about 10 kb, at least about 11 kb, at least about 12 kb, at least about 13 kb, at least about 14 kb, or at least about 15 kb. In another embodiment, the engineered nucleic acid processing enzyme reverse transcribes a RNA molecule that is at least about 7 kb or at least about 8 kb.

In some embodiments, the increase in thermoreactivity, resistance to reverse transcriptase inhibitors, ability to reverse transcribe difficult templates, speed, processivity, specificity, or sensitivity of the engineered nucleic acid processing enzyme as described herein has is about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 90%, or about 100% as compared to the wild-type polymerase.

E. Nucleic Acids and Expression Vectors

One aspect of the present disclosure provides an isolated nucleic acid molecule encoding the engineered thermostable reverse transcriptase or a derivatives thereof as described herein. In some embodiments, the engineered thermostable reverse transcriptase is encoded by a nucleic acid set forth herein or readily derived in light of polypeptide information provided herein and known in the art. The engineered thermostable reverse transcriptases need not be encoded by any specific nucleic acid exemplified herein. For example, redundancy in the genetic code allows for variations in nucleotide codon sequences that nevertheless encode the same amino acid. Accordingly, engineered polymerases of the present disclosure can be produced from nucleic acid sequences that are different from those set forth herein, for example, being codon optimized for a particular expression system. Codon optimization can be carried out, for example, as set forth in Athey et al., BMC Bioinformatics, 18:391-401 (2017).

Wild type polymerase nucleic acids may be isolated from naturally occurring sources to be used as starting material to generate novel polymerases. Generally, the nomenclature and the laboratory procedures in recombinant DNA technology described below are those well-known and commonly employed in the art. Standard techniques for cloning, DNA and RNA isolation, amplification and purification are known. Generally enzymatic reactions involving DNA ligase, DNA polymerase, restriction endonucleases are the like are performed according to the manufacturer's specifications. These techniques and various other techniques are generally performed according to Sambrook & Russell, Molecular Cloning—A Laboratory Manual, Cold Spring Harbor Laboratory, Cold Spring Harbor, N.Y., (1989) or Ausubel et al., Current Protocols in Molecular Biology, Vol. 1-3, John Wiley & Sons, Inc. (1994-1998).

The isolation of polymerase nucleic acids may be accomplished by a variety of techniques. The polymerase nucleic acids of the present invention can be generated from the wild type sequences. The wild type sequences are altered to create modified sequences. Wild type polymerases can be modified to create the polymerases claimed in the present application using methods that are well known in the art. Exemplary modification methods are site-directed mutagenesis, point mismatch repair, or oligonucleotide-directed mutagenesis.

Another aspect of the present disclosure provides an expression vector comprising the isolated nucleic acid encoding the engineered thermostable reverse transcriptase or derivatives thereof as described herein. A “vector” refers to a polynucleotide, which when independent of the host chromosome, is capable replication in a host organism. Preferred vectors include plasmids and typically have an origin of replication. Vectors can comprise, e.g., transcription and translation terminators, transcription and translation initiation sequences, and promoters useful for regulation of the expression of the particular nucleic acid. The polymerases of the present disclosure can be expressed in a variety of host cells, including E. coli, other bacterial hosts, yeasts, filamentous fungi, and various higher eukaryotic cells such as the COS, CHO and HeLa cells lines and myeloma cell lines. Techniques for gene expression in microorganisms are described in, for example, Smith, Gene Expression in Recombinant Microorganisms (Bioprocess Technology, Vol. 22), Marcel Dekker, 1994. Examples of bacteria that are useful for expression include, but are not limited to, Escherichia, Enterobacter, Azotobacter, Erwinia, Bacillus, Pseudomonas, Klebsielia, Proteus, Salmonella, Serratia, Shigella, Rhizobia, Vitreoscilla, and Paracoccus. Filamentous fungi that are useful as expression hosts include, for example, the following genera: Aspergillus, Trichoderma, Neurospora, Penicillium, Cephalosporium, Achlya, Podospora, Mucor, Cochliobolus, and Pyricularia. See, e.g., U.S. Pat. No. 5,679,543 and Stahl and Tudzynski, Eds., Molecular Biology in Filamentous Fungi, John Wiley & Sons, 1992. Synthesis of heterologous proteins in yeast is well known and described in the literature. Methods in Yeast Genetics, Sherman F. et al., Cold Spring Harbor Laboratory (1982) is a well-recognized work describing the various methods available to produce the enzymes in yeast. There are many expression systems for producing the polymerase polypeptides of the present invention that are well-known to those of ordinary skill in the art. See Gene Expression Systems, Fernandex and Hoeffler, Eds. Academic Press, 1999; Sambrook & Russell, supra; and Ausubel et al, Current Protocols in Molecular Biology, Vol. 1-3, John Wiley & Sons, Inc. (1994-1998).

Another aspect of the present disclosure provides a host cell transfected with the expression vector comprising the isolated nucleic acid encoding the engineered thermostable reverse transcriptase as described herein. Eukaryotic expression systems for mammalian cells, yeast, and insect cells are well known in the art and are also commercially available. In yeast, vectors include Yeast Integrating plasmids (e.g., YIp5) and Yeast Replicating plasmids (the YRp series plasmids) and pGPD-2. Expression vectors containing regulatory elements from eukaryotic viruses are typically used in eukaryotic expression vectors, e.g., SV40 vectors, papilloma virus vectors, and vectors derived from Epstein-Barr virus. Other exemplary eukaryotic vectors include pMSG, pAV009/A+, pMTO10/A+, pMAMneo-5, baculovirus pDSVE, and any other vector allowing expression of proteins under the direction of the CMV promoter, SV40 early promoter, SV40 later promoter, metallothionein promoter, murine mammary tumor virus promoter, Rous sarcoma virus promoter, polyhedrin promoter, or other promoters shown effective for expression in eukaryotic cells.

Once expressed, the engineered thermostable reverse transcriptase or a derivative thereof can be purified according to standard procedures of the art, including ammonium sulfate precipitation, affinity purification columns, column chromatography, gel electrophoresis and the like (see, generally, R. Scopes, Protein Purification, Springer-Verlag, N.Y. (1982), Deutscher, Methods in Enzymology Vol. 182: Guide to Protein Purification., Academic Press, Inc. N.Y. (1990)). Substantially pure compositions of at least about 90 to about 95% homogeneity are preferred, and about 98 to about 99% or more homogeneity are most preferred. Once purified, partially or to homogeneity as desired, the polypeptides may then be used (e.g., as immunogens for antibody production).

To facilitate purification of the engineered thermostable reverse transcriptase or a derivative thereof, the nucleic acids that encode the engineered thermostable reverse transcriptase or derivatives thereof can also include a coding sequence for an epitope or “tag” for which an affinity binding reagent is available. Examples of suitable epitopes include the myc and V-5 reporter genes; expression vectors useful for recombinant production of fusion polypeptides having these epitopes are commercially available (e.g., Invitrogen (Carlsbad Calif.) vectors pcDNA3.1/Myc-His and pcDNA3.1/V5-His are suitable for expression in mammalian cells). Additional expression vectors suitable for attaching a tag to the fusion proteins of the invention, and corresponding detection systems are known to those of skill in the art as described herein, and several are commercially available (e.g., FLAG″ (Kodak, Rochester N.Y.). Another example of a suitable tag is a polyhistidine sequence, which is capable of binding to metal chelate affinity ligands. Typically, six adjacent histidines are used (6His-tag, his-tag), although one can use more or less than six. Suitable metal chelate affinity ligands that can serve as the binding moiety for a polyhistidine tag include nitrilo-tri-acetic acid (NTA) (Hochuli, E. (1990) “Purification of recombinant proteins with metal chelating adsorbents” In Genetic Engineering: Principles and Methods, J. K. Setlow, Ed., Plenum Press, NY; commercially available from Qiagen (Santa Clarita, Calif.)).

One of skill in the art would recognize that after biological expression or purification, the engineered thermostable reverse transcriptase or derivatives thereof may possess a conformation substantially different than the native conformations of the constituent polypeptides. In this case, it may be necessary or desirable to denature and reduce the engineered thermostable reverse transcriptase or a derivative thereof and cause the engineered thermostable reverse transcriptase or a derivative thereof to re-fold into the preferred conformation. Methods of reducing and denaturing proteins and inducing re-folding are well known to those of skill in the art (See Debinski et al. (1993) J. Biol. Chem., 268: 14065-14070; Kreitman and Pastan (1993) Bioconjug. Chem., 4: 581-585; and Buchner et al. (1992) Anal. Biochem., 205: 263-270). Debinski et al., for example, describe the denaturation and reduction of inclusion body proteins in guanidine-DTE. The protein is then refolded in a redox buffer containing oxidized glutathione and L-arginine.

F. Compositions and Reaction Mixtures Comprising the Engineered Nucleic Acid Processing Enzyme or Derivatives Thereof

The present disclosure further provides compositions comprising a variety of components in various combinations needed for nucleic acid amplification. In some embodiments of the present disclosure, the compositions are formulated by admixing one or more engineered nucleic acid processing enzymes or derivatives thereof of the present disclosure in a buffered salt solution. One or more DNA polymerases and/or one or more nucleotides, and/or one or more primers may optionally be added to create the compositions of the invention. These compositions can be used in the methods disclosed herein to produce, analyze, quantitate and otherwise manipulate nucleic acid molecules (e.g., using reverse transcription or one-step RT-PCR procedures).

In some embodiments, the enzymes are provided at working concentrations (e.g., 1×) in stable buffered salt solutions. The terms “stable” and “stability” as used herein generally mean the retention by a composition, such as an enzyme composition, of at least 70%, preferably at least 80%, and most preferably at least 90%, of the original enzymatic activity (in units) after the enzyme or composition containing the enzyme has been stored for about one week at a temperature of about 4° C., about two to six months at a temperature of about −20° C., and about six months or longer at a temperature of about −80° C. As used herein, the term “working concentration” means the concentration of an enzyme that is at or near the optimal concentration used in a solution to perform a particular function such as reverse transcription of nucleic acids.

Such compositions can also be formulated as concentrated stock solutions (e.g., 2×, 3×, 4×, 5×, 6×, 10×, etc.). In some embodiments, having the composition as a concentrated (e.g., 5×) stock solution allows a greater amount of nucleic acid sample to be added (such as, for example, when the compositions are used for nucleic acid synthesis). The water used in forming the compositions of the present invention is preferably distilled, deionized and sterile filtered (through a 0.1-0.2 micrometer filter), and is free of contamination by DNase and RNase enzymes. Such water is available commercially, for example from Life Technologies (Carlsbad, Calif) or may be made as needed according to methods well known to those skilled in the art.

### III. Methods of Using the Engineered Nucleic Acid Processing Enzyme or a Derivative Thereof

A. Amplification Methods

Another aspect of the present disclosure provides a method of using the engineered nucleic acid processing enzyme or a derivative thereof as described herein, the method comprising contacting the engineered nucleic acid processing enzyme or a derivative thereof with a nucleic acid template under suitable conditions to produce a polymerized nucleic acid product. In some embodiments, the nucleic acid template is an RNA, a DNA, or a nucleic acid comprising an unnatural nucleotide.

The engineered nucleic acid processing enzyme or a derivative thereof as described herein may be used to make nucleic acid molecules from one or more templates. Such methods can comprise mixing one or more nucleic acid templates (e.g., DNA or RNA, such as non-coding RNA (ncRNA), messenger RNA (mRNA), micro RNA (miRNA), and small interfering RNA (siRNA) molecules) with one or more of the reverse transcriptases of the disclosure and incubating the mixture under conditions sufficient to generate one or more nucleic acid molecules complementary to all or a portion of the one or more nucleic acid templates. Other methods of cDNA synthesis which may advantageously use the present disclosure will be readily apparent to one of ordinary skill in the art.

In some embodiments, the method of using the engineered nucleic acid processing enzyme or a derivative thereof as described herein comprises the amplification of one or more nucleic acid molecules comprising mixing one or more nucleic acid templates with one of the engineered nucleic acid processing enzymes or fa derivative thereof of the disclosure, and incubating the mixture under conditions sufficient to amplify one or more nucleic acid molecules complementary to all or a portion of the one or more nucleic acid templates. In one embodiment, the method may further comprise the use of one or more DNA polymerases and may be employed as in standard reverse transcription-polymerase chain reaction (RT-PCR) reactions. In another embodiment, the method only comprises an engineered nucleic acid processing enzyme or a derivative thereof that functions in a single-step reverse transcription-polymerase chain reaction.

In some embodiments, the method of using the engineered nucleic acid processing enzyme or a derivative thereof as described herein may be one-step (e.g., one-step RT-PCR) or two-step (e.g., two-step RT-PCR) reactions. In one embodiment, the one-step RT-PCR type reactions may be accomplished in one tube thereby lowering the possibility of contamination. Such one-step reactions comprise (a) mixing a nucleic acid template (e.g., mRNA) with one or more engineered nucleic acid processing enzymes or derivatives thereof of the present disclosure and (b) incubating the mixture under conditions sufficient to amplify a nucleic acid molecule complementary to all or a portion of the template. Such amplification may be accomplished by the reverse transcriptase activity of the engineered nucleic acid processing enzyme alone or in combination with the DNA polymerase activity of the engineered nucleic acid processing enzyme.

In another embodiment, a two-step RT-PCR reaction may be accomplished in two separate steps. Such a method comprises (a) mixing a nucleic acid template (e.g., mRNA) with a engineered nucleic acid processing enzyme or a derivative thereof of the present disclosure, (b) incubating the mixture under conditions sufficient to make a nucleic acid molecule (e.g., a DNA molecule) complementary to all or a portion of the template, (c) mixing the nucleic acid molecule with one or more DNA polymerases and (d) incubating the mixture of step (c) under conditions sufficient to amplify the nucleic acid molecule. For amplification of long nucleic acid molecules (i.e., greater than about 3-5 kb in length), a combination of DNA polymerases and the engineered nucleic acid processing enzyme or a derivative thereof of the present disclosure may be used.

Amplification methods which may be used in accordance with the present invention (using one or more engineered nucleic acid processing enzymes or derivatives thereof of the present disclosure) include PCR, Isothermal Amplification, Strand Displacement Amplification (SDA), and Nucleic Acid Sequence-Based Amplification (NASBA); as well as more complex PCR-based nucleic acid fingerprinting techniques such as Random Amplified Polymorphic DNA (RAPD) analysis, Arbitrarily Primed PCR (AP-PCR) DNA Amplification Fingerprinting (DAF); microsatellite PCR; Directed Amplification of Minisatellite-region DNA (DAVID); digital droplet PCT (ddPCR) and Amplification Fragment Length Polymorphism (AFLP) analysis. See, e.g., EP 0 534 858; Vos, P., et al. Nucl. Acids Res. 23(21):4407-4414 (1995); Lin, J. J., and Kuo, J. FOCUS 17(2):66-70 (1995); U.S. Pat. Nos. 4,683,195 and 4,683,202; PCT Publication No. WO 2006/081222; U.S. Pat. No. 5,455,166; EP 0 684 315. U.S. Pat. No. 5,409,818; EP 0 329 822; Williams, J. G. K., et al., Nucl. Acids Res. 18(22):6531-6535, (1990); Welsh, J., and McClelland, M., Nucl. Acids Res. 18(24):7213-7218 (1990); Caetano-Anolles et al., Bio/Technology 9:553-557 (1991); Heath, D. D., et al. Nucl. Acids Res. 21(24): 5782-5785 (1993). Nucleic acid sequencing techniques which may employ the present compositions include dideoxy sequencing methods such as those disclosed in U.S. Pat. Nos. 4,962,022 and 5,498,523. In some embodiments, the invention may be used in methods of amplifying or sequencing a nucleic acid molecule comprising one or more polymerase chain reactions (PCRs), such as any of the PCR-based methods described above.

Methods of producing an engineered thermostable reverse transcriptase or a derivative thereof of the present disclosure are known to those of skill in the art of molecular biology or molecular genetics. For example, nucleic acids encoding the wild type polymerase or nucleic acid binding domains can be generated using routine techniques in the field of recombinant genetics. Basic texts disclosing the general methods of use in this invention include Sambrook and Russell, Molecular Cloning, A Laboratory Manual (3rd ed. 2001); Kriegler, Gene Transfer and Expression: A Laboratory Manual (1990); Current Protocols in Molecular Biology (Ausubel et al., eds., 1994-1999); Berger, Sambrook, and Ausubel, as well as Mullis et al., (1987) U.S. Pat. No. 4,683,202; PCR Protocols A Guide to Methods and Applications (Innis et al., eds) Academic Press Inc. San Diego, Calif. (1990) (Innis); Arnheim & Levinson (Oct. 1, 1990) C&EN 36-47; The Journal Of NIH Research (1991) 3: 81-94; (Kwoh et al. (1989) Proc. Natl. Acad. Sci. USA 86: 1173; Guatelli et al. (1990) Proc. Natl. Acad. Sci. USA 87, 1874; Lomell et al. (1989) J. Clin. Chem., 35: 1826; Landegren et al., (1988) Science 241: 1077-1080; Van Brunt (1990) Biotechnology 8: 291-294; Wu and Wallace (1989) Gene 4: 560; and Barringer et al. (1990) Gene 89: 117.

B. Nucleic Acid Sample Processing

One aspect of the present disclosure provides a nucleic acid extension method comprising contacting a target nucleic acid molecule with an engineered nucleic acid processing enzyme or a derivative thereof and a plurality of nucleic acid barcoded molecules comprising a barcode sequence (e.g., a capture probe), and incubating the target nucleic acid, the engineered nucleic acid processing enzyme or a derivative thereof and barcoded molecules under conditions in which the barcoded molecules are extended by the engineered nucleic acid processing enzyme. The target nucleic acid hybridizes to one of the plurality of barcoded molecules and the hybridized barcoded molecule is extended by the engineered thermostable reverse transcriptase using the target nucleic acid as a template, thereby creating a first strand nucleic acid (e.g. cDNA). In some embodiments, the engineered nucleic acid processing enzyme comprises a first domain comprising a polymerase domain, and a second domain conjugated to the first domain. In some embodiments, the polymerase domain comprises an amino acid sequence of an engineered Thermococcus gorgonarius polymerase (Tgo polymerase); and the second domain comprises a nucleic acid binding domain. In some embodiments, one of the plurality of nucleic acid barcoded molecules hybridizes to the target nucleic acid molecule. In some embodiments, the nucleic acid binding domain interacts with and stabilizes the target nucleic acid molecule and/or primer or barcoded molecule complex while the polymerase domain extends one of the plurality of nucleic acid barcoded molecules that is hybridized to the target nucleic acid molecule.

1. RNA Template

In some embodiments, the nucleic acid is a ribonucleic acid (RNA) molecule; and the engineered nucleic acid processing enzyme reverse transcribes the RNA molecule thereby generating a first strand cDNA, and subsequently or concurrently amplifies the cDNA into a nucleic acid product in the same reaction. In one embodiment, the RNA molecule is a messenger RNA (mRNA) molecule.

In some embodiments of the nucleic acid extension method as described herein, each of the plurality of nucleic acid barcoded molecules comprises a molecular tag. Molecular tags include unique molecular identifiers (UMIs) and the UMIs comprise a polynucleotide. In some embodiments, the nucleic acid barcoded molecules further comprise capture sequences. A capture sequence can comprise a random N-mer sequence wherein the random N-mer sequence is complementary to a 3′ sequence of the RNA molecules. In some embodiments, the capture sequence comprises a poly-dT sequence having a length of at least 5 bases. In some embodiments, the capture sequence comprises a poly-dT sequence having a length of at least 10 bases. In some embodiments, the capture sequence comprises a poly-dT sequence having a length of at least 5 bases, at least 6 bases, at least 7 bases, at least 8 bases, at least 9 bases, at least 10 bases.

In some embodiments, a reverse transcription reaction of the engineered nucleic acid processing enzyme of the present disclosure is initiated at the point of hybridization of the capture sequences to the RNA molecules, with the capture probe being extended by the engineered nucleic acid processing enzyme of the present disclosure in a template directed fashion using the hybridized mRNA as a template. In some embodiments, the reverse transcription reaction produces single stranded cDNA molecules each having a molecular tag and barcode associated with the cDNA, followed by amplification of cDNA to produce a double stranded cDNA that includes the sequences of the barcoded molecules.

In some embodiments, the plurality of nucleic acid barcoded molecules comprises an oligo(dT) sequence. In that embodiment, the engineered nucleic acid processing enzyme reverse transcribes the mRNA molecule into a complementary DNA molecule using the mRNA hybridized to the oligo(dT) sequence of the nucleic acid barcoded molecules as a template, and the nucleic acid binding domain binds and stabilizes the mRNA-oligo(dT) hybrid during the reverse transcription. Following reverse transcription, the engineered nucleic acid processing enzyme as described herein further amplifies the complementary DNA molecule comprising the barcode sequence, thereby generating an amplified DNA product comprising the barcode sequence, molecular tag sequence, or complements thereof.

In some embodiments of the nucleic acid extension method described herein, the method further comprises a second nucleic acid molecule comprising an oligo(dT) sequence. In that embodiment, the plurality of nucleic acid barcoded molecules further comprise an oligo(dT) sequence; and the nucleic acid binding domain of the engineered nucleic acid processing enzyme binds and stabilizes the mRNA-Oligo(dT) hybrid, while the polymerase domain of the engineered nucleic acid processing enzyme reverse transcribes the mRNA molecule using the second nucleic acid molecule comprising the oligo(dT) sequence, thereby generating a complementary DNA molecule. In this embodiment, the engineered nucleic acid processing enzyme further amplifies the complementary DNA molecule, thereby generating an amplified DNA product comprising a barcode sequence.

In some embodiments, the nucleic acid extension method further comprises a cell, a population of cells, or a tissue and the template nucleic acid molecule is from the cell, population of cells or the tissue.

2. Volume

In some embodiments, the engineered reverse transcriptase enzymes or derivatives thereof as described herein are used in a reaction volume less than about 1 nanoliter (nL). In some embodiments, the engineered reverse transcriptase enzymes or derivatives thereof as described herein are used in a reaction volume is less than about 500 picoliter (pL). In some embodiments, the reaction volume is contained within a partition. In some embodiments, the reaction volume is contained within a droplet. In some embodiments, the reaction volume is contained within a droplet in an emulsion. In some embodiments, the reaction volume is contained within a droplet emulsion having a reaction volume of less than about 1 nL. In some embodiments, the reaction volume is contained within a droplet emulsion having a reaction volume of less than about 500 pL. In some embodiments, the reaction volume is contained within a well. In some embodiments, the reaction volume is contained within a well having a reaction volume less than about 1 nL. In some embodiments, the reaction volume is contained within a well. In some embodiments, the reaction volume is contained within a well having a reaction volume less than about 500 pL. In some embodiments, the reaction volume is contained within a well in an array of wells having an extracted nucleic acid molecule, and wherein the template nucleic acid molecule is the extracted nucleic acid molecule. In some embodiments, the reaction volume is contained within a well in an array of wells having a cell comprising a template nucleic acid molecule, and wherein the template nucleic acid molecule is released from the cell.

3. Gel Bead

In some embodiments of the nucleic acid extension method described herein, the plurality of nucleic acid barcoded molecules are attached to a support (e.g. a particle, a slide, a chip, a bead, etc.). In one embodiment, the support is selected from the group consisting of an array, a bead, a gel bead, a microparticle, and a polymer. In some embodiments, the nucleic acid barcoded molecules attached to a support comprise molecular tags (UMIs), primer sequences, capture sequences, cleavage sequences, or additional functional sequences. In some embodiments, the support is a gel bead. In that embodiment, the nucleic acid barcoded molecules are releasably attached to the gel bead. In some embodiments, the gel bead comprises a polyacrylamide polymer.

In some embodiments, a cross-section of the gel bead is less than about 100 μm. In some embodiments, a cross-section of a gel bead is less than about 60 μm. In some embodiments, a cross-section of a gel bead is less than about 50 μm. In some embodiments, a cross-section of a gel bead is less than about 40 μm. In some embodiments, a cross-section of a gel bead is less than about 100 μm, less than about 99 μm, less than about 98 μm, less than about 97 μm, less than about 96 μm, less than about 95 μm, less than about 94 μm, less than about 93 μm, less than about 92 μm, less than about 91 μm, less than about 90 μm, less than about 89 μm, less than about 88 μm, less than about 87 μm, less than about 86 μm, less than about 85 μm, less than about 84 μm, less than about 83 μm, less than about 82 μm, less than about 81 μm, less than about 80 μm, less than about 79 μm, less than about 78 μm, less than about 77 μm, less than about 76 μm, less than about 75 μm, less than about 74 μm, less than about 73 μm, less than about 72 μm, less than about 71 μm, less than about 70 μm, less than about 69 μm, less than about 68 μm, less than about 67 μm, less than about 66 μm, less than about 65 μm, less than about 64 μm, less than about 63 μm, less than about 62 μm, less than about 61 μm, or less than about 60 am.

Functionalization of beads for attachment of nucleic acid molecules (e.g., oligonucleotides) may be achieved through a wide range of different approaches, including activation of chemical groups within a polymer, incorporation of active or activatable functional groups in the polymer structure, or attachment at the pre-polymer or monomer stage in bead production.

For example, precursors (e.g., monomers, cross-linkers) that are polymerized to form a bead may comprise acrydite moieties, such that when a bead is generated, the bead also comprises acrydite moieties. The acrydite moieties can be attached to a nucleic acid molecule (e.g., oligonucleotide), which may include a priming sequence (e.g., a primer for amplifying target nucleic acids, random primer, primer sequence for messenger RNA) and/or one or more barcode sequences. The one more barcode sequences may include sequences that are the same for all nucleic acid molecules coupled to a given bead and/or sequences that are different across all nucleic acid molecules coupled to the given bead. The nucleic acid molecule may be incorporated into the bead.

In some cases, the nucleic acid molecule can comprise a functional sequence, for example, for attachment to a sequencing flow cell, such as, for example, a P5 sequence for Illumina® sequencing. In some cases, the nucleic acid molecule or derivative thereof (e.g., oligonucleotide or polynucleotide generated from the nucleic acid molecule) can comprise another functional sequence, such as, for example, a P7 sequence for attachment to a sequencing flow cell for Illumina sequencing. In some cases, the nucleic acid molecule can comprise a barcode sequence. In some cases, the primer can further comprise a unique molecular identifier (UMI). In some cases, the primer can comprise an R1 sequence for use in Illumina sequencing workflows. In some cases, the primer can comprise an R2 sequence for use in Illumina sequencing workflows. Examples of such nucleic acid molecules (e.g., oligonucleotides, polynucleotides, etc.) and uses thereof, as may be used with compositions, devices, methods and systems of the present disclosure, are provided in U.S. Patent Pub. Nos. 2014/0378345 and 2015/0376609, each of which is entirely incorporated herein by reference. However, the present invention is not limited as to a composition of any nucleic acid molecule or derivative thereof, or any particular sequencing platform and these characterizations serve as examples only which may be useful in a reverse transcription workflow.

In operation, a cell can be co-partitioned along with a barcode bearing bead. The barcoded nucleic acid molecules affixed to a bead can be released from the bead in the partition. By way of example, in the context of analyzing sample RNA, the poly-dT (poly-deoxythymine, also referred to as oligo (dT)) segment of one of the released nucleic acid molecules can hybridize to (e.g., capture) the poly-A tail of a mRNA molecule. Reverse transcription may result in a cDNA transcript of the mRNA which cDNA transcript also includes each of the sequence segments of the nucleic acid molecule. Because the nucleic acid molecule comprises additional functional sequences (e.g., capture domains, primer domains, UMIs, barcodes, etc.), it can hybridize to and prime reverse transcription of the mRNA using the hybridized mRNA as a template. Within any given partition, all of the cDNA transcripts of the individual mRNA molecules may include a common barcode sequence. However, the transcripts made from the different mRNA molecules within a given partition may vary with respect to unique molecular identifying sequences (e.g., UMIs). Beneficially, following any subsequent amplification of the contents of a given partition, the number of different UMIs can be indicative of the quantity of mRNA originating from a given partition, and thus from the cell. As noted above, the transcripts can be amplified and sequenced to identify the sequence of the original mRNA captured template, as well as the sequence of the associated barcode and UML. While a poly-dT capture sequence is described, other targeted or random capture sequences may also be used in capture or hybridize to a template for initiating the reverse transcription reaction.

4. Engineered Nucleic Acid Processing Enzyme

In some embodiments of the nucleic acid extension method disclosed herein, the engineered nucleic acid processing enzyme comprises the amino acid sequence set forth in SEQ ID NO: 1, which is the sequence of the wild-type T. gorgonarius polymerase (WT Tgo, TgoPol). In one embodiment, the engineered nucleic acid processing enzyme comprises a sequence having at least about 90% sequence identity to the amino acid sequence set forth in SEQ ID NO: 1. In some embodiments, the engineered nucleic acid processing enzyme comprises an amino acid sequence having at least about 90%, at least about 91%, at least about 92%, at least about 93%, at least about 94%, at least about 95%, at least about 96%, at least about 97%, at least about 98%, or at least about 99% sequence identity to the amino acid sequence of SEQ ID NO: 1. In one embodiment, the engineered nucleic acid processing enzyme comprises an amino acid sequence having at least 95% identity to the amino acid sequence of SEQ ID NO: 1. In one embodiment, the engineered nucleic acid processing enzyme comprises an amino acid sequence having at least 97% identity to the amino acid sequence of SEQ ID NO: 1. In another embodiment, the engineered nucleic acid processing enzyme comprises an amino acid sequence having at least about 10, at least about 15, or at least about 20 substitutions in the amino acid sequence of SEQ ID NO: 1. In one embodiment, the engineered nucleic acid processing enzyme comprises an amino acid sequence having at least 97% identity to the amino acid sequence of SEQ ID NO: 1 and at least about 15 substitutions in the amino acid sequence of SEQ ID NO: 1.

In some embodiments of the engineered nucleic acid processing enzyme described herein, the polymerase domain comprises: (a) an aspartic acid substitution at position 141; (b) a glutamic acid substitution at position 143; (c) an alanine substitution at position 485; (d) a valine substitution at position 93; (e) an arginine substitution at position 97; (f) a tyrosine substitution at position 384; (g) a valine substitution at position 389; (h) a phenylalanine substitution at position 493; (i) a phenylalanine substitution at position 587; (j) a glutamic acid substitution at position 664; (k) a glycine substitution at position; (l) a tryptophan substitution at position 768; (m) an isoleucine substitution at position 2; (n) an isoleucine substitution at position 38; (o) a lysine substitution at position 118; (p) a methionine substitution at position 137; (q) an arginine substitution at position 381; (r) a lysine substitution at position 466; (s) a tyrosine substitution at position 514; (t) an isoleucine substitution at position 521; and/or (u) an asparagine substitution at position 735 of SEQ ID NO: 1.

In some embodiments of the nucleic acid extension methods disclosed herein, the polymerase domain of the engineered nucleic acid processing enzyme as described herein comprises a substitution selected from the group consisting of an aspartic acid to alanine substitution at position 141 (D141A); a glutamic acid to alanine substitution at position 143 (E143A); an alanine to leucine substitution at position 485 (A485L); a valine to glutamine substitution at position 93 (V93Q); an arginine to methionine substitution at position 97 (R97M); a tyrosine to histidine substitution at position 384 (Y384H); a valine to isoleucine substitution at position 389 (V389I); a phenylalanine to leucine substitution at position 493 (F493L); a phenylalanine to leucine substitution at position 587 (F587L); a glutamic acid to lysine substitution at position 664 (E664K); a glycine to valine substitution at position (G711V); a tryptophan to arginine substitution at position 768 (W768R); an isoleucine to valine substitution at position 2 (I2V); an isoleucine to leucine substitution at position 38 (I38L); a lysine to isoleucine substitution at position 118 (K118I); a methionine to leucine substitution at position 137 (M137L); an arginine to histidine substitution at position 381 (R381H); a lysine to arginine substitution at position 466 (K466R); a tyrosine to isoleucine substitution at position 514 (T514I); an isoleucine to leucine substitution at position 521 (I521L); and an asparagine to lysine substitution at position 735 (N735K) of SEQ ID NO: 1.

In one embodiment of the nucleic acid extension methods disclosed herein, the polymerase domain of the engineered nucleic acid processing enzyme as described herein comprises at least one, at least two, at least three, at least five, at least six, at least seven, at least eight, at least nine, at least ten, at least eleven, at least twelve, at least fifteen, or at least twenty of the substitution disclosed herein.

In one embodiment of the nucleic acid extension methods disclosed herein, the polymerase domain of the engineered nucleic acid processing enzyme described herein comprises a combination of R97M, D141A, E143A, Y384H, V389I, Y493L, F587L, E664K, G711V, and W768R substitutions in SEQ ID NO: 1. In another embodiment, the polymerase domain of the engineered nucleic acid processing enzyme described herein comprises I2V, I38L, R97M, K118I, M137L, E143A, R381H; Y384H, V389I, K466R, F493L, T514I, I521L, F587L, E664K, G711V, N735K, and W768R substitutions in SEQ ID NO: 1. In another embodiment, the polymerase domain of the engineered nucleic acid processing enzyme described herein comprises I2V, I38L, R97M, K118I, M137L, D141A, E143A, R381H, Y384H, V389I, K466R, F493L, T514I, I521L, F587L, E664K, G711V, N735K, and W768R substitutions in SEQ ID NO: 1. Alternatively, the polymerase domain of the engineered nucleic acid processing enzyme described herein comprises I2V, 138L, V93Q, R97M, K118I, M137L, D141A, E143A, R381H, Y384H, A485L, V389I, K466R, F493L, T514I, I521L, F587L, E664K, G711V, N735K, and W768R substitutions in SEQ ID NO: 1.

In some embodiments of the nucleic acid extension methods disclosed herein, the engineered nucleic acid processing enzyme comprises the amino acid sequence of SEQ ID NO: 2 or 3.

In some embodiments of the nucleic acid extension methods disclosed herein, the engineered nucleic acid processing enzyme of the present disclosure is conjugated to a nucleic acid binding domain. A DNA binding domain is a protein, or a defined region of a protein, that binds to nucleic acid in a sequence-independent matter. For example, binding of the protein to DNA does not exhibit any preference for a particular sequence. In some embodiments, the DNA binding domain may be single or double stranded. In one embodiment, the nucleic acid binding domain comprises a single stranded DNA binding protein; a double stranded DNA binding protein; a single stranded RNA binding protein; a double stranded RNA binding protein; a continuous RNA-DNA hybrid binding protein; or a discontinuous RNA-DNA hybrid binding protein. In some embodiments, the nucleic acid binding domain helps stabilize the interaction between the RNA template and the DNA primer during reverse transcription. In one embodiment, the nucleic acid binding domain enhances the efficiency and/or processivity of the engineered thermostable enzyme during reverse transcription. In some embodiments, a suitable DNA binding domain for the present disclosure will be identical to or substantially identical to a known DNA binding protein over a comparison window of about 25 amino acids, about 50-100 amino acids, or over the length of the entire protein. The sequence can be compared and aligned for maximum correspondence over a comparison window, or designated region as measured using one of the described comparison algorithms or by manual alignment and visual inspection. For purposes of this patent, percent amino acid identity is determined by the default parameters of BLAST or CLUSTAL W.

In some embodiments of the nucleic acid extension methods disclosed herein, the nucleic acid binding domain comprises a nucleic acid binding protein selected from the group consisting of a histone-like protein, an archaeal basic nucleic acid binding protein, a basic DNA binding domain, HMf-like protein, Proliferating cell nuclear antigen (PCNA), HU-like protein, HU-family DNA binding protein, Sm-like protein domain; HU, sto7, Sso7d, Sac7d, and Sac7e. In one embodiment, the nucleic acid binding domain comprises a histone-like protein. In one embodiment, the nucleic acid binding domain comprises a bacterial histone-like protein or a bacterial HU-family DNA binding protein.

As disclosed herein, HU family DNA-binding protein is a conserved nucleoid-associated protein that binds non-specifically to duplex DNA with a particular preference for targeting nicked and bent DNA. HU is highly basic and contributes to chromosomal compaction and maintenance of negative supercoiling, thus often referred to as histone-like protein. In one embodiment, the nucleic acid binding domain is a Thermus thermophile HU-family DNA binding protein. The conjugation or fusion of HU to the recombinant thermostable polymerase of the present disclosure is a novel approach of solving the problem associated with the analysis of the transcriptome or medical diagnostics using RNA because HU has a dual DNA and RNA binding properties as disclosed herein.

Accordingly, in an embodiment of the present disclosure, the nucleic acid binding domain of the engineered nucleic acid processing enzyme binds a DNA and RNA hybrid complex. The DNA-RNA hybrid can be continuous or discontinuous. As described herein, the terms continuous and discontinuous in the context of nucleic acid replication is well known to those skilled in the art.

In some embodiments, the nucleic acid binding protein comprises an amino acid sequence having at least 90%, at least 91%, at least 92%, at least 93%, at least 94%, at least 95%, at least 96%, at least 97%, at least 98%, or at least 99% sequence identity to the amino acid sequence of SEQ ID NO: 6. In some embodiments, the nucleic acid binding protein comprises an amino acid sequence having 90%, 91%, 92%, 93%, 94%, 95%, 96%, 97%, 98%, or 99% sequence identity to the amino acid sequence of SEQ ID NO: 6. In another embodiment, the nucleic acid binding domain comprises an amino acid sequence set forth in SEQ ID NO: 6 or an amino acid sequence having at least 90% sequence identity to the amino acid sequence set forth in SEQ ID NO: 6.

In another embodiment of the nucleic acid extension methods disclosed herein, the engineered nucleic acid processing enzyme comprises an amino acid sequence of SEQ ID NO: 5; or an amino acid sequence having at least 90% sequence identity to the amino acid sequence set forth in SEQ ID NO: 5. In some embodiments, the engineered nucleic acid processing enzyme comprises an amino acid sequence of SEQ ID NO: 4; or an amino acid sequence having at least 90% sequence identity to the amino acid sequence of SEQ ID NO: 4.

In yet another embodiment of the nucleic acid extension methods disclosed herein, the engineered nucleic acid processing enzyme has a dual DNA and RNA polymerase activity, or an RNA reverse transcriptase activity.

In another embodiment of the nucleic acid extension methods disclosed herein, the engineered nucleic acid processing enzyme or a derivative thereof further comprises a tag protein selected from the group consisting of an affinity tag, a fluorescent tag, or an expression and/or solubility enhancement tag. In some embodiments, the tag is selected from hexahistidine tag (his-tag), small ubiquitin-like modifier tag (SUMO), aVariFlex C-Terminal solubility enhancement tag, a short peptide C-terminal tag, Thioredoxin (Trx) tag, aVariFlex C-Terminal solubility enhancement tag, Solubility-enhancer peptide sequences (SET) tag, IgG domain B1 of Protein G (GB1) tag, IgG repeat domain ZZ of Protein A (ZZ) tag, Solubility enhancing Ubiquitous Tag (SNUT tag), Seventeen kilodalton protein (Skp tag), Phage T7 protein kinase (T7PK) tag, E. coli secreted protein A (EspA) tag, Monomeric bacteriophage T7 0.3 protein (Orc protein) (Mocr) tag, E. coli trypsin inhibitor (Ecotin) tag, Calcium-binding protein (CaBP) tag, Stress-responsive arsenate reductase (ArsC) tag, N-terminal fragment of translation initiation factor IF2 (IF2-domain I) tag, N-terminal fragment of translation initiation factor IF2 (Expressivity) tag, Fasciola hepatica 8-kDa antigen tag (Fh8), Glutathione-S-transferase (GST) tag, maltose-binding protein tag (MBP), FLAg tag peptide (FLAG), streptavidin binding peptide tag (Strep-II; strep), calmodulin-binding protein tag (CBP), mutated dehalogenase tag (HaloTag), staphylococcal Protein A (Protein A), intein mediated purification with the chitin-binding domain (IMPACT (CBD)), cellulose-binding module (CBM), dockerin domain of Clostridium josui tag (Dock), fungal avidin-like protein (Tamavidin).

In one embodiment, the tag is an affinity tag selected from hexahistidine tag (his-tag), Fasciola hepatica 8-kDa antigen tag (Fh8), Glutathione-S-transferase (GST) tag, maltose-binding protein tag (MBP), FLAg tag peptide (FLAG), streptavidin binding peptide tag (Strep-II), calmodulin-binding protein tag (CBP), mutated dehalogenase tag (HaloTag), staphylococcal Protein A (Protein A), intein mediated purification with the chitin-binding domain (IMPACT (CBD)), cellulose-binding module (CBM), dockerin domain of Clostridium josui tag (Dock), fungal avidin-like protein (Tamavidin). In one embodiment, the tag is hexahistidine tag (his-tag). In such an embodiment, the engineered nucleic acid processing enzyme comprises an amino acid sequence of SEQ ID NO: 9.

In another embodiment of the nucleic acid extension methods disclosed herein, the engineered nucleic acid processing enzyme is thermophilic; and/or is resistant to thermal inactivation when compared to a wild-type polymerase from which the engineered thermostable reverse transcriptase is derived. The engineered nucleic acid processing enzyme may be resistant to thermal inactivation at a temperature from about 53° C. to about 75° C.; from about 55° C. to about 75° C.; from about 60° C. to about 75° C.; from about 53° C. to about 68° C.; from about 55° C. to about 68° C.; from about 45° C. to about 68° C.; or from about 50° C. to about 68° C. Alternatively, the engineered nucleic acid processing enzyme may be resistant to thermal inactivation at a temperature of about 68° C.

In another embodiment of the nucleic acid extension methods disclosed herein, the engineered nucleic acid processing enzyme possesses enhanced half-life when compared to a wild-type polymerase from which the engineered thermostable reverse transcriptase is derived at a temperature from about 53° C. to about 75° C.; from about 55° C. to about 75° C.; from about 60° C. to about 75° C.; from about 53° C. to about 68° C.; from about 55° C. to about 68° C.; from about 45° C. to about 68° C.; or from about 50° C. to about 68° C. The determination of the half-life of the t engineered nucleic acid processing enzyme is described herein and is known to those of skill in the art. In some embodiments, the engineered nucleic acid processing enzyme possesses one or more of the following characteristics when compared to a wild-type polymerase from which the engineered thermostable reverse transcriptase is derived: increased thermostability; increased thermoreactivity; increased resistance to reverse transcriptase inhibitors; increased ability to reverse transcribe difficult templates; increased speed; increased processivity; increased specificity; enhanced polymerization activity; or increased sensitivity. Each of these characteristics is described and defined herein.

In some embodiments, the increase in thermoreactivity, resistance to reverse transcriptase inhibitors, ability to reverse transcribe difficult templates, speed, processivity, specificity, or sensitivity is enhanced by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 90%, or about 100% as compared to the wild-type polymerase. In another embodiment, the polymerization activity is enhanced by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 90%, or about 100% as compared to the wild-type polymerase.

In some embodiments, the engineered nucleic acid processing enzyme reverse transcribes a RNA molecule having at least about 100, at least about 200, at least about 300, at least about 400, at least about 500, at least about 600, at least about 700, at least about 800, at least about 900, or at least about 1000 nucleotides. In another embodiment, the engineered nucleic acid processing enzyme reverse transcribes a RNA molecule that is at least about 1 kb, at least about 2 kb, at least about 3 kb, at least about 4 kb, at least about 5 kb, at least about 6 kb, at least about 7 kb, at least about 8 kb, at least about 9 kb, at least about 10 kb, at least about 11 kb, at least about 12 kb, at least about 13 kb, at least about 14 kb, or at least about 15 kb. In yet another embodiment, the engineered nucleic acid processing enzyme reverse transcribes an RNA molecule that is at least about 7 kb or at least about 8 kb.

5. PCNA Enhances the Activity of the Recombinant Thermostable Polymerase Enzyme

As described herein, many but not all family B DNA polymerases interact with accessory proteins to achieve highly processive DNA synthesis. Clamp proteins or clamps were initially identified as the processivity factor of replicative DNA polymerases, but it is now known that these molecules are indispensable for the timely and faithful replication of a DNA genome. Proliferating cell nuclear antigen (PCNA) physically interacts with a large group of proteins and coordinates their functions in various DNA amplification reactions. See Zhihao Zhuang and Yongxing Ai, Biochim Biophys Acta., 1804(5): 1081-1093 (2010).

The addition of a T. kodakarensis PCNA (KPCNA) to a nucleic acid extension reaction enhanced the reverse transcriptase activity of the thermostable chimeric polymerase enzyme described herein (e.g., TgoRT or TgoRTxo) in a dose dependent manner (FIG. 4). In the presence of KPCNA, the reverse transcriptase activity of TgoRTxo was enhanced by at least a factor of four. See also Example 3. Accordingly, in some embodiments of the nucleic acid extension methods disclosed herein, a nucleic acid binding domain, such as PCNA, is fused to the recombinant thermostable polymerase enzyme. In some embodiments, the engineered thermostable reverse transcriptase comprises a PCNA. In one embodiment, the PCNA is a T. kodakarensis PCNA.

Alternatively, in some embodiments of the nucleic acid extension methods disclosed herein, the target nucleic acid molecule is further contacted with a sliding clamp molecule. The sliding clamp can be any sliding clamp known in the art. In one embodiment, the sliding clamp molecule is an archea, eucarya, or a bacteriophage sliding clamp protein. In one embodiment, the sliding clamp protein is selected from E. coli polymerase β subunit; T4 bacteriophage gp45, T. gorgonarius PCNA, or T. kodakarensis PCNA. In one embodiment, the sliding clamp protein is T. kodakarensis PCNA.

Additional methods and systems for characterizing nucleic acids from small populations of cells, and in some cases, for characterizing nucleic acids from individual cells, especially in the context of larger populations of cells using the engineered thermostable reverse transcriptase of the present disclosure are known to those of skill in the art. See e.g., U.S. Patent Publication Nos. 2015/0376609, 2019/0367997; 2019/0064173, and 2021/0115415; and International Application Nos. PCT/US2020/17785, and PCT/US2018/016019. The methods and systems provide advantages of being able to provide the attribution advantages of the non-amplified single molecule methods with the high throughput of the other next generation systems, with the additional advantages of being able to process and sequence extremely low amounts of input nucleic acids derivable from individual cells or small collections of cells.

### IV. Kits

One aspect of the present invention provides a kit comprising the engineered nucleic acid processing enzyme or a derivative thereof as described herein. In some embodiments, the kit further comprises one or more of a vector, a nucleotide, a buffer, a salt, and/or instructions. In another embodiment, a kit may comprise an engineered nucleic acid processing enzyme or a derivative thereof for use in reverse transcription or amplification of a nucleic acid molecule. In yet another embodiment, a kit may be used for single cell profiling of the transcriptome. In yet another embodiment, a kit may be used for spatial transcriptomics methods and assays. In yet another embodiment, a kit may be used for in situ methods and assays.

The kit may include suitable reaction buffers, dNTPs, one or more primers, one or more control reagents, or any other reagents disclosed for performing the methods of the present disclosure. The engineered nucleic acid processing enzyme or a derivative thereof, reaction buffer, and dNTPs may be provided separately or may be provided together in a master mix solution. When the engineered nucleic acid processing enzyme or a derivative thereof, reaction buffer, and dNTPs are provided in a master mix, the master mix is present at a concentration at least two times the working concentration indicated in instructions for use in an extension reaction. In other cases, the master mix may be present at a concentration at least three times, at least four times, at least five times, at least six times, at least seven times, at least eight times, at least nine times, or at least ten times, the working concentration indicated. The primer in the kits may be a poly-dT primer, a random N-mer primer, or a target-specific primer.

The kits may further include one, two, three, four, five or more, up to all of partitioning fluids, including both aqueous buffers and non-aqueous partitioning fluids or oils, nucleic acid barcode capture probes that are releasably associated with beads, as described herein, microfluidic devices, reagents for disrupting cells, reagents for amplifying nucleic acids, as well as instructions for using any of the foregoing in the methods described herein.

The instructions for using any of the methods are generally recorded on a suitable recording medium (e.g. printed on a substrate such as paper or plastic), or available in a digital format. As such, the instructions may be present in the kits as a package insert, in the labeling of the container of the kit or components thereof (i.e., associated with the packaging or subpackaging). In some cases, the instructions may be present as an electronic storage data file present on a suitable computer readable storage medium. In other cases, the actual instructions may not be present in the kit, but means for obtaining the instructions from a remote source, e.g. via the internet, may be provided. For example, a kit that includes a web address where the instructions may be viewed and/or from which the instructions may be downloaded. As with the instructions, this means for obtaining the instructions is recorded on a suitable substrate.

Kits according to this aspect of the invention comprise a carrier means, such as a box, carton, tube or the like, having in close confinement therein one or more container means, such as vials, tubes, ampoules, bottles and the like, wherein a first container means contains one or more of the engineered nucleic acid processing enzymes or derivatives thereof of the present disclosure having reverse transcriptase activity. When more than one polypeptide having reverse transcriptase activity is used, they may be in a single container as mixtures of two or more engineered nucleic acid processing enzymes or derivatives thereof, or in separate containers. The kits of the disclosure can also comprise (in the same or separate containers) one or more DNA polymerases, a suitable buffer, one or more nucleotides and/or one or more primers.

The kits of the disclosure can also comprise one or more hosts or cells including those that are competent to take up nucleic acids (e.g., DNA molecules including vectors). Preferred hosts may include chemically competent or electrocompetent bacteria such as E. coli (including DH5, DH5a, DH10B, HB101, Top 10, and other K-12 strains as well as E. coli B and E. coli W strains).

In a specific aspect of the disclosure, the kits of the disclosure (e.g., reverse transcription and amplification kits) can include one or more components (in mixtures or separately) including one or more engineered nucleic acid processing enzymes or derivative thereof having reverse transcriptase activity of the disclosure, one or more nucleotides (one or more of which may be labeled, e.g., fluorescently labeled) used for synthesis of a nucleic acid molecule, and/or one or more primers (e.g., oligo(dT) for reverse transcription, randomers for extension reactions, etc). Such kits can further comprise one or more DNA polymerases.

### V. Definitions

Unless defined otherwise, all technical and scientific terms used herein generally have the same meaning as commonly understood by one of ordinary skill in the art to which this technology belongs. As used in this specification and the appended claims, the singular forms “a”, “an” and “the” include plural referents unless the content clearly dictates otherwise. “A and/or B” is used herein to include all of the following alternatives: “A”, “B”, “A or B”, and “A and B”. For example, reference to “a cell” includes a combination of two or more cells, and the like. Generally, the nomenclature used herein and the laboratory procedures in cell culture, molecular genetics, organic chemistry, analytical chemistry and nucleic acid chemistry and hybridization described below are those well-known and commonly employed in the art.

Where values are described as ranges, it will be understood that such disclosure includes the disclosure of all possible sub-ranges within such ranges, as well as specific numerical values that fall within such ranges irrespective of whether a specific numerical value or specific sub-range is expressly stated.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number. If the degree of approximation is not otherwise clear from the context, “about” means either within plus or minus 10% of the provided value, or rounded to the nearest significant figure, in all cases inclusive of the provided value. In some embodiments, the term “about” indicates the designated value ± up to 10%, up to ±5%, or up to ±1%.

Headings, e.g., (a), (b), (i) etc., are presented merely for ease of reading the specification and claims. The use of headings in the specification or claims does not require the steps or elements be performed in alphabetical or numerical order or the order in which they are presented.

Use of ordinal terms such as “first”, “second”, “third”, etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements. Similarly, the use of these terms in the specification does not by itself connote any required priority, precedence, or order.

By “Analyte” is intended a biological molecule. Analytes include but are not limited to a DNA analyte, an RNA analyte, an oligonucleotide, a reporter molecule, a reporter molecule configured to directly couple to a protein, a reporter molecule configured to indirectly couple to a protein, a reporter molecule configured to directly couple to a metabolite, and a reporter molecule configured to indirectly couple to a metabolite.

The terms “adaptor(s)”, “adapter(s)” and “tag(s)” may be used synonymously. An adaptor or tag can be coupled to a polynucleotide sequence to be “tagged” by any approach, including ligation, hybridization, or other approaches.

The term “sequencing,” as used herein, generally refers to methods and technologies for determining the sequence of nucleotide bases in one or more polynucleotides. The polynucleotides can be, for example, nucleic acid molecules such as deoxyribonucleic acid (DNA) or ribonucleic acid (RNA), including variants or derivatives thereof (e.g., single stranded DNA). Sequencing can be performed by various systems currently available, such as, without limitation, a sequencing system by Illumina®, Pacific Biosciences (PacBio®), Oxford Nanopore®, or Life Technologies (Ion Torrent®). Alternatively or in addition, sequencing may be performed using nucleic acid amplification, polymerase chain reaction (PCR) (e.g., digital PCR, quantitative PCR, or real time PCR), or isothermal amplification. Such systems may provide a plurality of raw genetic data corresponding to the genetic information of a subject (e.g., human), as generated by the systems from a sample provided by the subject. In some examples, such systems provide sequencing reads (also “reads” herein). A read may include a string of nucleic acid bases corresponding to a sequence of a nucleic acid molecule that has been sequenced. In some situations, systems and methods provided herein may be used with proteomic information.

The term “bead,” as used herein, generally refers to a particle. The bead may be a solid or semi-solid particle. The bead may be a gel bead. The gel bead may include a polymer matrix (e.g., matrix formed by polymerization or cross-linking). The polymer matrix may include one or more polymers (e.g., polymers having different functional groups or repeat units). Polymers in the polymer matrix may be randomly arranged, such as in random copolymers, and/or have ordered structures, such as in block copolymers. Cross-linking can be via covalent, ionic, or inductive, interactions, or physical entanglement. The bead may be a macromolecule. The bead may be formed of nucleic acid molecules bound together. The bead may be formed via covalent or non-covalent assembly of molecules (e.g., macromolecules), such as monomers or polymers. Such polymers or monomers may be natural or synthetic. Such polymers or monomers may be or include, for example, nucleic acid molecules (e.g., DNA or RNA). The bead may be formed of a polymeric material. The bead may be magnetic or non-magnetic. The bead may be rigid. The bead may be flexible and/or compressible. The bead may be disruptable or dissolvable. The bead may be a solid particle (e.g., a metal-based particle including but not limited to iron oxide, gold or silver) covered with a coating comprising one or more polymers. Such coating may be disruptable or dissolvable.

As used herein, the term “barcoded nucleic acid molecule” generally refers to a nucleic acid molecule that results from, for example, the processing of a nucleic acid barcoded molecule with a nucleic acid sequence (e.g., nucleic acid sequence complementary to a nucleic acid primer sequence encompassed by the nucleic acid barcoded molecule). The nucleic acid sequence may be a targeted sequence or a non-targeted sequence. The nucleic acid barcoded molecule may be coupled to or attached to the nucleic acid molecule comprising the nucleic acid sequence. For example, a nucleic acid barcoded molecule described herein may be hybridized to an analyte (e.g., a messenger RNA (mRNA) molecule) of a cell. Reverse transcription can generate a barcoded nucleic acid molecule that has a sequence corresponding to the nucleic acid sequence of the mRNA and the barcode sequence (or a reverse complement thereof). The processing of the nucleic acid molecule comprising the nucleic acid sequence, the nucleic acid barcoded molecule, or both, can include a nucleic acid reaction, such as, in non-limiting examples, reverse transcription, nucleic acid extension, ligation, etc. The nucleic acid reaction may be performed prior to, during, or following barcoding of the nucleic acid sequence to generate the barcoded nucleic acid molecule. For example, the nucleic acid molecule comprising the nucleic acid sequence may be subjected to reverse transcription and then be attached to the nucleic acid barcoded molecule to generate the barcoded nucleic acid molecule, or the nucleic acid molecule comprising the nucleic acid sequence may be attached to the nucleic acid barcoded molecule and subjected to a nucleic acid reaction (e.g., extension, ligation) to generate the barcoded nucleic acid molecule. A barcoded nucleic acid molecule may serve as a template, such as a template polynucleotide, that can be further processed (e.g., amplified) and sequenced to obtain the target nucleic acid sequence. For example, in the methods and systems described herein, a barcoded nucleic acid molecule may be further processed (e.g., amplified) and sequenced to obtain the nucleic acid sequence of the nucleic acid molecule (e.g., mRNA).

The term “sample,” as used herein, generally refers to a biological sample of a subject. The biological sample may comprise any number of macromolecules, for example, cellular macromolecules. The sample may be a cell sample. The sample may be a cell line or cell culture sample. The sample can include one or more cells. The sample can include one or more microbes. The biological sample may be a nucleic acid sample or protein sample. The biological sample may also be a carbohydrate sample or a lipid sample. The biological sample may be derived from another sample. The sample may be a tissue sample, such as a biopsy, core biopsy, needle aspirate, or fine needle aspirate. The sample may be a fluid sample, such as a blood sample, urine sample, or saliva sample. The sample may be a skin sample. The sample may be a cheek swab. The sample may be a plasma or serum sample. The sample may be a cell-free or cell free sample. A cell-free sample may include extracellular polynucleotides. Extracellular polynucleotides may be isolated from a bodily sample that may be selected from the group consisting of blood, plasma, serum, urine, saliva, mucosal excretions, sputum, stool and tears.

The term “subject,” as used herein, generally refers to an animal, such as a mammal (e.g., human) or avian (e.g., bird), or other organism, such as a plant. For example, the subject can be a vertebrate, a mammal, a rodent (e.g., a mouse), a primate, a simian or a human. Animals may include, but are not limited to, farm animals, sport animals, and pets. A subject can be a healthy or asymptomatic individual, an individual that has or is suspected of having a disease (e.g., cancer) or a pre-disposition to the disease, and/or an individual that is in need of therapy or suspected of needing therapy. A subject can be a patient. A subject can be a microorganism or microbe (e.g., bacteria, fungi, archaea, viruses).

The term “molecular tag,” as used herein, generally refers to a molecule capable of binding to a macromolecular constituent. The molecular tag may bind to the macromolecular constituent with high affinity. The molecular tag may bind to the macromolecular constituent with high specificity. The molecular tag may comprise a nucleotide sequence. The molecular tag may comprise a nucleic acid sequence. The nucleic acid sequence may be at least a portion or an entirety of the molecular tag. The molecular tag may be a nucleic acid molecule or may be part of a nucleic acid molecule. The molecular tag may be an oligonucleotide or a polypeptide. The molecular tag may comprise a DNA aptamer. The molecular tag may be or comprise a primer. The molecular tag may be, or comprise, a protein. The molecular tag may comprise a polypeptide. The molecular tag may be a barcode.

The term “partition,” as used herein, generally, refers to a space or volume that may be suitable to contain one or more species or conduct one or more reactions. A partition may be a physical compartment, such as a droplet or well. The partition may isolate space or volume from another space or volume. The droplet may be a first phase (e.g., aqueous phase) in a second phase (e.g., oil) immiscible with the first phase. The droplet may be a first phase in a second phase that does not phase separate from the first phase, such as, for example, a capsule or liposome in an aqueous phase. A partition may comprise one or more other (inner) partitions. In some cases, a partition may be a virtual compartment that can be defined and identified by an index (e.g., indexed libraries) across multiple and/or remote physical compartments. For example, a physical compartment may comprise a plurality of virtual compartments.

The term “partitioning” as used herein is intended to encompass parting, dividing, depositing, separating, or compartmentalizing into one or more partitions. Systems and methods for partitioning of one or more particles (such as, but not limited to, biological particles, macromolecular constituents of biological particles, beads, reagents, etc.) into discrete compartments or partitions (referred to interchangeably here as partitions), wherein each partition maintains separation of its own content from the contents of other partitions are known in the art. See for example US 2020/0032335, herein incorporated by reference in its entirety. The partition can be a droplet in an emulsion. A partition may comprise one or more other partitions.

A “plurality of nucleic acid barcoded molecules” may comprise at least about 500 nucleic acid barcoded molecules, at least about 1,000 nucleic acid barcoded molecules, at least about 5,000 nucleic acid barcoded molecules, at least about 10,000 nucleic acid barcoded molecules, at least about 50,000 nucleic acid barcoded molecules, at least about 100,000 nucleic acid barcoded molecules, at least about 500,000 nucleic acid barcoded molecules, at least about 1,000,000 barcoded molecules, at least about 5,000,000 nucleic acid barcoded molecules, at least about 10,000,000 nucleic acid barcoded molecules, at least about 100,000,000 nucleic acid barcoded molecules, at least about 1,000,000,000 nucleic acid barcoded molecules. In some cases, a plurality of nucleic acid barcoded molecules comprise a partition-specific barcode sequence.

Each of the plurality of nucleic acid barcoded molecules may include an identifier sequence separate from the partition-specific barcode sequence, where the identifier sequence is different for each nucleic acid partition-specific barcoded molecule of the plurality of nucleic acid partition specific barcoded molecules. In some cases, such an identifier sequence is a unique molecular identifier (UMI) as described elsewhere herein. As described elsewhere herein, UMI sequences can uniquely identify a particular nucleic acid molecule that is barcoded, which may be identifying particular nucleic acid molecules that are analyzed, counting particular nucleic acid molecules that are analyzed, etc. Furthermore, in some cases, each of the plurality of nucleic acid barcoded molecules can comprise the partition specific barcode sequence and the bead can be from plurality of beads, such as a population of barcoded beads. Each of the partition specific barcode sequences can be different from partition specific barcode sequences of nucleic acid barcoded molecules of other beads of the plurality of beads. Where this is the case, a population of barcoded beads, with each bead comprising a different partition specific barcode sequence can be analyzed.

As used herein, the terms “unique molecular identifier”, “unique molecular identifying sequence”, “UMI” and “UMI sequence” are used synonymously. Individual barcoded molecules may comprise a common barcode sequence such as a partition specific sequence or a spatial array where every capture probe has a unique barcode sequence.

By “binding sequence” is intended a nucleic acid sequence capable of binding to an analyte.

A nucleic acid barcoded molecule of a plurality of nucleic acid molecules may be used to generate a “barcoded nucleic acid molecule.” In some cases, a barcoded molecule comprises a different reporter barcode sequence that identifies a second analyte. A different reporter barcode sequence or an analyte-specific barcode sequence may identify a protein, a lipid, a metabolite or other second analyte.

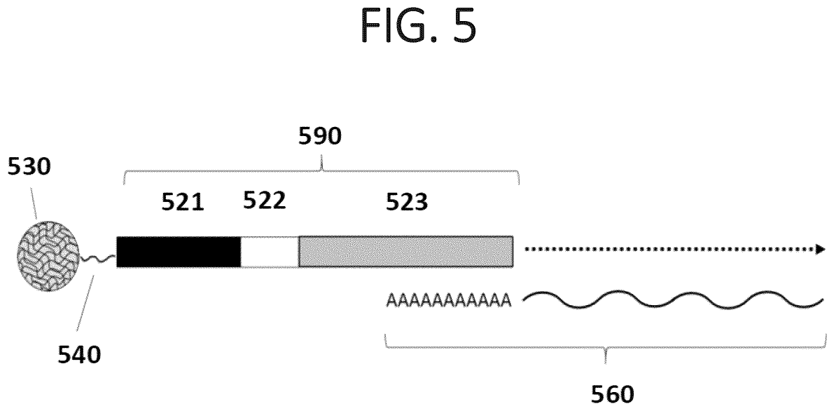

Barcoded nucleic acids may be generated (e.g., via a nucleic acid reaction, such as nucleic acid extension or ligation) from the constructs described in FIG. 5. For example, capture handle sequence may then be hybridized to complementary sequence, such as capture sequence 523 to generate (e.g., via a nucleic acid reaction, such as nucleic acid extension or ligation) a barcoded nucleic acid molecule comprising cell (e.g., partition specific) barcode sequence 522 (or a reverse complement thereof) and reporter barcode sequence 522 (or a reverse complement thereof). In some embodiments capture handle sequence 523 comprises a sequence complementary to a template switching oligonucleotide on the capture sequence 523. In some embodiments, the nucleic acid barcoded molecule 590 (e.g., partition-specific barcoded molecule) further includes a UMI (not shown). Barcoded nucleic acid molecules can then be optionally processed as described elsewhere herein, e.g., to amplify the molecules and/or append sequencing platform specific sequences to the fragments. See, e.g., U.S. Pat. Pub. 2018/0105808, which is hereby entirely incorporated by reference for all purposes. Barcoded nucleic acid molecules, or derivatives generated therefrom, can then be sequenced on a suitable sequencing platform.

In some instances, analysis of multiple analytes (e.g., nucleic acids and one or more analytes using labelling agents described herein) may be performed. In some instances, analysis of an analyte (e.g. a nucleic acid, a polypeptide, a carbohydrate, a lipid, a glycan, a glycan motif, a metabolite, a protein, etc.) comprises a workflow as generally depicted in FIG. 5. A nucleic acid barcoded molecule 590 (e.g. partition specific barcoded molecule) may be co-partitioned with the one or more analytes. In some instances, nucleic acid barcoded molecule 590 is attached to a support 530 (e.g., a bead, such as a gel bead), such as those described elsewhere herein. For example, nucleic acid barcoded molecule 590 may be attached to support 530 via a releasable linkage 540 (e.g., comprising a labile bond), such as those described elsewhere herein. Nucleic acid barcoded molecule 590 may comprise a functional sequence 521 and optionally comprise other additional sequences, for example, a barcode sequence 522 (e.g., common barcode, partition-specific barcode, or other functional sequences described elsewhere herein), and/or a UMI sequence (not shown). The nucleic acid barcoded molecule 590 may comprise a capture sequence 523 that may be complementary to another nucleic acid sequence, such that it may hybridize to a particular sequence, e.g., capture handle sequence 523.

For example, capture sequence 523 may comprise a poly-T sequence and may be used to hybridize to mRNA. Referring to FIG. 5, in some embodiments, nucleic acid barcoded molecule 590 comprises capture sequence 523 complementary to a sequence of RNA molecule 560 from a cell. In some instances, capture sequence 523 comprises a sequence specific for an RNA molecule. Capture sequence 523 may comprise a known or targeted sequence or a random sequence. In some instances, a nucleic acid extension reaction may be performed, thereby generating a barcoded nucleic acid product comprising capture sequence 523, the functional sequence 521, barcode sequence 522, any other functional sequence, and a sequence corresponding to the RNA molecule 560.

In another example, capture sequence 523 may be complementary to an overhang sequence or an adapter sequence that has been appended to an analyte. Any suitable agent may degrade beads. Suitable agents may include, but are not limited to, changes in temperature, changes in pH, reduction, oxidation and exposure to water or other aqueous solutions.

In some instances, a cell that is bound to labelling agent which is conjugated to oligonucleotide and support 530 (e.g., a bead, such as a gel bead) comprising nucleic acid barcoded molecule 590 is partitioned into a partition amongst a plurality of partitions (e.g., a droplet of a droplet emulsion or a well of a microwell array).

As used herein, the term “operably linked” or “conjugated” or “fusion” means that, in relation to the recombinant thermostable polymerase enzyme sequence there are one or more sequences at the N or C terminus that, when transcribed and translated, create additional polypeptides in association with the enzyme amino acid sequence, thereby created a conjugation or fusion of one or more polypeptides from one expression vector.

As used herein, the term “reverse transcriptase activity,” “reverse transcription activity,” or “reverse transcription” indicates the capability of an enzyme to synthesize a DNA strand (that is, complementary DNA or cDNA) using RNA as a template.

As used herein, the term “mutation” or “mutant” or “variant” indicates a change or changes introduced in a wild type DNA sequence or a wild type amino acid sequence. Examples of mutations or variants include, but are not limited to, substitutions, insertions, deletions, and point mutations. Mutations can be made either at the nucleic acid level or at the amino acid level.

As used herein, the term “thermoreactivity” or “thermoreactive” refers to the ability of a reverse transcriptase to exhibit enzyme activity at elevated temperatures.

As used herein, “thermostability” or “thermostable” refers to the ability of a reverse transcriptase to withstand exposure to elevated temperatures, but not necessarily show activity at such elevated temperatures. In some embodiments, thermostable reverse transcriptase or polymerase refers to any enzyme that catalyzes polynucleotide synthesis by addition of nucleotide units to a nucleotide chain using DNA or RNA as a template and has an optimal activity at a temperature above 53° C.

As used herein, the term “processivity” refers to the ability of a reverse transcriptase to continuously extend a primer without disassociating from the nucleic acid template. The length of a template a reverse transcriptase or polymerase is capable of replicating can also be used to describe the processivity of that reverse transcriptase or polymerase. In some embodiments, “Processivity” refers to the ability of a polymerase to remain bound to the template or substrate and perform DNA synthesis. Processivity is measured by the number of catalytic events that take place per binding event.

As used herein, the term “inhibitor resistance” refers to the ability of a reverse transcriptase to perform reverse transcription in the presence of a compound, chemical, protein, buffer, etc. that is typically inhibitory to the reverse transcriptase (prevents or inhibits reverse transcriptase activity).

As used herein, the term “fidelity” refers to the accuracy of polymerization, or the ability of the reverse transcriptase to discriminate correct from incorrect substrates, (e.g., nucleotides) when synthesizing nucleic acid molecules which are complementary to a template. The higher the fidelity of a reverse transcriptase, the less the reverse transcriptase misincorporates nucleotides in the growing strand during nucleic acid synthesis; that is, an increase or enhancement in fidelity results in a more faithful reverse transcriptase having decreased error rate or decreased misincorporation rate.

As used herein, the term “identical” in the context of two nucleic acids or polypeptide sequences refers to the residues in the two sequences that are the same when aligned for maximum correspondence, as measured using a sequence comparison algorithms. Sequence comparison algorithms are known to those skill in the art. See. E.g., ebi.ac.uk/Tools/msa/clustalo/.

As used herein, the term “efficiency” in the context of a nucleic acid modifying enzyme of this invention refers to the ability of the enzyme to perform its catalytic function under specific reaction conditions. Typically, “efficiency” as defined herein is indicated by the amount of product generated under given reaction conditions.

As used herein, the term “enhances” in the context of an enzyme refers to improving the activity of the enzyme, i.e., increasing the amount of product per unit enzyme per unit time.

### EXAMPLES

The present technology is further illustrated by the following Examples, which should not be construed as limiting in any way. The examples herein are provided to illustrate advantages of the present technology and to further assist a person of ordinary skill in the art with preparing or using the compositions and systems of the present technology. The examples should in no way be construed as limiting the scope of the present technology, as defined by the appended claims. The examples can include or incorporate any of the variations, aspects, or embodiments of the present technology described above. The variations, aspects, or embodiments described above may also further each include or incorporate the variations of any or all other variations, aspects or embodiments of the present technology.

### Example 1: An Engineered Thermophilic Reverse Transcriptase

This example demonstrates preparation of an engineered thermophilic reverse transcriptase T. gorgonarius (Tgo) polymerase that is more efficient at reverse transcription at high temperatures and in small volume as compared to prior known engineered reverse transcriptases.

Most known natural and engineered reverse transcriptases are active at temperatures ranging from 37° C. to 55° C. This narrow temperature range of activity makes it difficult to combine a reverse transcription reaction (RTx) with a DNA amplification reaction (PCR) in the same reaction vessel and in the absence of a second polymerase. In addition, use of a second polymerase in the same reaction mixture as the RT reaction has other consequences for the quality of the data generated. For instance, it was observed while performing experiments for this disclosure that the sensitivity and specificity of these reactions were generally lesser than those using the present invention and were further more time consuming.

To overcome these limitations, a thermophilic polymerase enzyme was engineered to also have a reverse transcriptase activity at higher temperature.

A person of skill in the art, using the engineered polymerase of the present disclosure would be able to utilize a single amplification reaction to generate a nucleic acid amplification product (DNA) by first generating a cDNA from mRNA and then amplifying that cDNA within a single reaction mixture using the engineered reverse transcriptase/polymerase enzyme of the present disclosure. This one-step reaction has many advantages. For example, a thermophilic enzyme with dual reverse transcriptase and DNA polymerase activity would negate the need for two separate reactions, thereby streamlining RT-PCR assays, high throughput amplification reaction assays (e.g. spatial array and single cell transcriptomics assay) and the like.

Previously, a DNA polymerase from Thermococcus kodakarensis (KOD polymerase SEQ ID NO: 7) was engineered to perform reverse transcription at 68° C. Using a DNA polymerase from a Thermococcus gorgonarius (Tgo polymerase; SEQ ID NO: 1), a reverse transcriptase was engineered by rational design, that was capable of reverse transcribing RNA at temperature ranging from 37° C. to 70° C. This rationale design identified a group of 20 amino acids that were important for generating the reverse transcriptase activity: I2V, 138L, R97M, K118I, M137L, R381H, Y384H, V389I, K466R, F493L, T514I, I521L, F587L, E664K, G711V, N735K, and W768R.

The engineered thermophilic enzyme functioned as a DNA polymerase and was capable of amplifying DNA. The dual RT/DNA polymerase activity was demonstrated by showing that the engineered thermophilic enzyme amplified DNA products following PCR amplification of a sample comprising only an RNA template. Furthermore, the engineered thermophilic enzyme transcribed an RNA and generated an amplification product at low (53° C.) and high (68° C.) temperatures. (FIG. 1B and FIG. 2B, peak at ˜1300 at arrow). In contrast, a control Moloney Murine Leukemia Virus (MMLV) reverse-transcriptase (MMLV RT) variant reverse transcribed that same RNA at low temperatures (53° C.), but failed to reverse transcribe that RNA at high temperature (68° C.) (FIG. 1A and FIG. 2A). The engineered thermophilic polymerase enzyme disclosed herein demonstrated greater efficiency at reverse transcribing long RNA molecules (1300 nt) at temperatures ranging from 53° C. to 68° C. as compared to the control MMLV variant RT enzyme. In fact, the relative amount of product generated using the MMLV RT enzyme is about half (approximately 600) when compared to the TgoRTxo product generation (approximately 1200). In addition, the TgoRTxo product generation was increased at 68° C. over that seen at 53° C.

TgoRT and TgoRTox are More Efficient than a MMLV RT Variant Enzyme for RNA Analysis of Droplets of Less than 1 nL.

A clear body of evidence demonstrated that reverse transcription of mRNA from a single cell was inhibited from an unknown component(s) present in a cell lysate when the reaction volume was less than about 1 nL. To overcome this inhibition and facilitate the utilization of smaller reaction volumes, the control MMLV RT variant enzyme was tested in droplets containing picoliter-sized reaction volumes. The control MMLV RT enzyme variant effectively reduced the previously identified inhibition of reverse transcription in a 350 pL reaction volume in comparison to a second available mutant MMLV RT enzyme. However, the observation that TgoRT and TgoRTox were more efficient at high temperatures than either MMLV RT enzymes attested to the novelty and unexpected effect of the engineered enzymes in single cell analysis of RNA in small volume.

In addition to the thermophilic Tgo enzyme that is exonuclease proficient (TgoRT; SEQ ID NO: 2), a thermophilic Tgo enzyme that was exonuclease deficient (TgoRTxo; SEQ ID NO: 3) was engineered.

TgoRT and TgoRTox are More Efficient than Corresponding T. kodakarensis Enzymes

The engineered thermophilic T. gorgonarius reverse transcriptase was found during experimentation to be more efficient at reverse transcribing a template than engineered reverse transcriptases known in the art. For example, a DNA polymerase from Thermococcus kodakarensis (KOD polymerase; SEQ ID NO: 7) was engineered to reverse transcribe RNA. See e.g., Elefson et al Science 336(6079): 341-344 (2016). This reverse transcriptase engineered from the backbone of KOD DNA polymerase generated cDNA from RNA substrates using regular amplification techniques. When tested in a high throughput system, such as spatial array transcriptomics assay, single cell transcriptomics assay, a single cell profiling reaction, or related single cell sequencing system, the efficiency of the engineered KOD polymerase (KODRTx) was less than that seen from the TgoRTx disclosed herein. The KodRTx enzyme that was known in the art was unable to reverse transcribe an RNA template at 53° C. However, the engineered TgoRTxo of the present disclosure showed robust activity at 53° C. (FIG. 1B, at arrow). Indeed, the reverse transcriptase efficiency of the engineered TgoRTxo at 53° C. was equal to or perhaps more efficient at transcribing a 1300 nt template compared to a variant Moloney Murine Leukemia Virus (MMLV) reverse-transcriptase (MMLV RT) enzyme (FIG. 1A, at arrow). A sequence comparison showed that wild type T. gorgonarius DNA polymerase (TgoPol) is about 92.63% identical to wild type T. kodakarensis polymerase (KodPol) (FIG. 3). While not being bound to any particular theory, it is possible that T. gorgonarius DNA polymerase is a better enzyme for high throughput amplification assays such as the single cell analysis or cellular RNA analysis using droplets in emulsion. In addition, T. gorgonarius DNA polymerase may be more efficiency for RNA analysis in a volume (e.g., droplet) that is less than 1 nL.

This example demonstrates that a reverse transcriptase engineered using a polymerase from T gorgonarius as a backbone was very efficient at reverse transcribing long RNA molecules (1300 nt) at temperatures ranging from 53° C. to 68° C. The engineered Tgo reverse transcriptase enzyme was able to survive prolonged heating at high temperatures and could also function as a polymerase enzyme for amplifying DNA (e.g. PCR, extension reactions, etc.). The thermophilic enzyme disclosed herein can be engineered to either have proofreading capability (TgoRT) or not (TgoRTxo).

### Example 2: PCNA to Improve Reverse Transcription

This example demonstrates that the reverse transcriptase activity of an engineered TgoRTxo is enhanced and improved in the presence of a Proliferating Cell Nuclear Antigen (PCNA).

PCNA molecules, found in nuclei of yeast, plant and animal cells exhibit a three part function—a sliding clamp during DNA synthesis, a polymerase switch factor and as a recruitment factor for DNA repair proteins/enzymes. Strazalka & Zimienowicz, Ann. Bot. 107(7):1127-40 (2011). PCNA is an essential component in the eukaryotic DNA replication machinery, where it works to tether DNA polymerases on the DNA template to enhance processive DNA synthesis. To date, all known replicative DNA polymerases, including the archeal family B DNA polymerases, are thought to require a sliding clamp. For example, the E. coli DNA polymerase uses the β subunit of DNA pol as a sliding clamp; the bacteriophage T4 uses gp45, and eucarya and archaea use PCNA. Indeed, an archea PCNA from Aeropyrum pernix has been shown to interact with DNA polymerases of that organism to augment DNA synthesis by these cognate DNA polymerases in vitro. Daimon et al., J. Bacteriol. 184(3):687-94 (2002).

To determine whether the activity of the engineered reverse transcriptase of the present disclosure (TgoRTxo) would be enhanced by a sliding clamp protein, the effects of the T. kodakarensis proliferating cell nuclear antigen (KPCNA) in combination with TgoRTxo was tested in vitro. Recombinant KPCNA was expressed in E. coli and purified to homogeneity. Varying concentrations of purified KPCNA were added to reverse transcription reaction mixtures containing: a ˜600 nt RNA template, DNA primer, TgoRTxo Reverse Transcriptase, Mg, dNTPs, buffer. The concentration of KPCNA was increased from 0 to 1 μM, keeping all other reagents constant.

As shown in FIG. 4, the inclusion of KPCNA in an RT reaction enhanced the reverse transcriptase activity of TgoRTxo. As the concentration of KPCNA was increased from 0 to 1 uM, the efficiency of reverse transcription was increased by about a factor of 4. The reverse transcriptase efficiency of TgoRTxo was enhanced from about 10% in the absence of KPCNA to about 45% with the addition of 1 μM KPCNA. (FIG. 4). Therefore, in the presence of 1 μM KPCNA, the activity of TgoRTxo RT was enhanced by at least a factor of four. As FIG. 4 demonstrates, at 3.5 μM KPCNA, the effect of KPCNA on the RT reaction efficiency was less than that seen at 1 μM, however even at 3.5 μM KPCNA, the RT efficiency of TgoRTxo was approximately 3 times higher as compared to no KPCNA.

During reverse transcription, supplementing the reaction mixture containing RT enzyme with KPCNA improved the RT efficiency. While not being bound by any particular theory, it is contemplated that the increase in efficiency may be the result of: (1) the KPCNA molecule stabilizing the primer-RNA hybrid and/or (2) the KPCNA acting to recruit the TgoRTxo to the primer-RNA junction followed by enzyme stabilization.

Accordingly, this example shows that the engineered TgoRT functions as a polymerase enzyme and can interact with a sliding clamp enzyme in vitro. Further, this example clearly shows that the PCNA molecule helps improve the reverse transcription efficiency of the engineered TgoRTxo. While PCNA molecules from other organisms have been studied in the context of DNA amplification in PCR reactions, PCNA molecules have not been evaluated in reverse transcription reactions.

This disclosure provides the first demonstration that a PCNA molecule can enhance the activity of a reverse transcriptase. In addition to the embodiment provided herein, KPCNA can be further included at the C- or N-terminus on a protein or protein fusion with, for example, a hexahistidine tag (His tag) or any purification tags disclosed herein to facilitate its purification.

### Example 3: Fusion of TgoRT or TgoRTxo to DNA Binding Proteins for Improved RT Efficiency

This example demonstrates that the fusion of the recombinant thermophilic polymerase to a HU domain enhances the processivity of the engineered nucleic acid processing enzyme.

It was contemplated that fusing an HU domain to the C-terminus of the recombinant thermophilic polymerase might help stabilize the DNA primer binding to RNA (DNA-RNA hybrids) and enhance the reverse transcription efficiency and processivity of the engineered nucleic acid processing enzyme.

Sequential PCR is used to fuse the T. thermophile HU-family DNA binding protein (Huth) to the C-terminus of the recombinant Tgo polymerase using molecular techniques that are known to those of skilled in the art. In addition, the Huth is engineered to introduce mutations in the HU protein that enhance the primer-template binding specificity of the HU-TgoRT fusion protein.

In addition to HUth, other molecules may be fused to the engineered reverse transcriptase to enhance its activity. Specifically, the KPCNA molecule may be fused to the C terminus or N terminus of the engineered reverse transcriptase (TgoRT) to enhance the processivity of the TgoRT, with or without a HUth or other DNA binding domain, as disclosed in Example 2 above.

### EQUIVALENTS

The present technology is not to be limited in terms of the particular embodiments described in this application, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the present technology. It is to be understood that this present technology is not limited to particular methods, reagents, compounds compositions or biological systems, which can, of course, vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

All patents, patent applications, provisional applications, and publications referred to or cited herein are incorporated by reference in their entirety, including all figures and tables, to the extent they are not inconsistent with the explicit teachings of this specification.

### INCORPORATION BY REFERENCE

All publications, patents, and patent applications mentioned in this specification are herein incorporated by reference in their entireties to the same extent as if each individual publication, patent, or patent application was specifically and individually indicated to be incorporated by reference.

What is claimed is:

1. An engineered nucleic acid processing enzyme comprising:
(a) a first domain comprising a polymerase domain, wherein the polymerase domain comprises an amino acid sequence of an engineered Thermococcus gorgonarius polymerase (Tgo polymerase); and
(b) a second domain conjugated to the first domain, wherein the second domain comprises a nucleic acid binding domain.

2. The engineered nucleic acid processing enzyme of claim 1, wherein the polymerase domain comprises an amino acid sequence having:
(a) at least 90%, at least 91%, at least 92%, at least 93%, at least 94%, at least 95%, at least 96%, at least 97%, at least 98%, or at least 99% sequence identity to the amino acid sequence of SEQ ID NO: 1;
(b) at least 95% identity to the amino acid sequence of SEQ ID NO: 1;
(c) at least 97% identity to the amino acid sequence of SEQ ID NO: 1;
(d) at least about 10, at least about 15, or at least about 20 substitutions in the amino acid sequence of SEQ ID NO: 1;
(e) at least 97% identity to the amino acid sequence of SEQ ID NO: 1 and at least about 15 substitutions in the amino acid sequence of SEQ ID NO: 1; or
(f) 90%, 91%, 92%, 93%, 94%, 95%, 96%, 97%, 98%, 99% sequence identity to the amino acid sequence of SEQ ID NO: 1.

3. The engineered nucleic acid processing enzyme of claim 2, wherein the polymerase domain comprises: (a) an aspartic acid substitution at position 141; (b) a glutamic acid substitution at position 143; (c) an alanine substitution at position 485; (d) a valine substitution at position 93; (e) an arginine substitution at position 97; (f) a tyrosine substitution at position 384; (g) a valine substitution at position 389; (h) a phenylalanine substitution at position 493; (i) a phenylalanine substitution at position 587; (j) a glutamic acid substitution at position 664; (k) a glycine substitution at position; (l) a tryptophan substitution at position 768; (m) an isoleucine substitution at position 2; (n) an isoleucine substitution at position 38; (o) a lysine substitution at position 118; (p) a methionine substitution at position 137; (q) an arginine substitution at position 381; (r) a lysine substitution at position 466; (s) a tyrosine substitution at position 514; (t) an isoleucine substitution at position 521; and/or (u) an asparagine substitution at position 735 of SEQ ID NO: 1.

4. The engineered nucleic acid processing enzyme of claim 2, wherein:
(a) the polymerase domain comprises a substitution at positions 141 and 143 of SEQ ID NO: 1 or 2;
(b) the polymerase domain comprises a substitution at position 141 of SEQ ID NOs: 2;
(c) the engineered nucleic acid processing enzyme lacks proofreading activity; or
(d) the engineered nucleic acid processing enzyme comprises the amino acid sequence of SEQ ID NO: 1, 2 or 3.

5. The engineered nucleic acid processing enzyme of claim 2, wherein the polymerase domain comprises a combination of:
(a) R97M, D141A, E143A, Y384H, V389I, Y493L, F587L, E664K, G711V, and W768R substitutions in SEQ ID NO: 1;
(b) I2V, I38L, R97M, K118I, M137L, E143A, R381H; Y384H, V389I, K466R, F493L, T514I, I521L, F587L, E664K, G711V, N735K, and W768R substitutions in SEQ ID NO: 1;
(c) I2V, I38L, R97M, K118I, M137L, D141A, E143A, R381H, Y384H, V389I, K466R, F493L, T514I, I521L, F587L, E664K, G711V, N735K, and W768R substitutions in SEQ ID NO: 1; or
(d) I2V, I38L, V93Q, R97M, K118I, M137L, D141A, E143A, R381H, Y384H, A485L, V389I, K466R, F493L, T514I, I521L, F587L, E664K, G711V, N735K, and W768R substitutions in SEQ ID NO: 1.

6. The engineered nucleic acid processing enzyme of claim 1, wherein the nucleic acid binding domain comprises:
(a) a nucleic acid binding protein selected from the group consisting of a histone-like protein, an archaeal basic nucleic acid binding protein, a basic DNA binding domain, HMf-like protein, HU-like protein, HU-family DNA binding protein, Sm-like protein domain, proliferating cell nuclear antigen (PCNA), HU, sto7, Sso7d, Sac7d, and Sac7e;
(b) a T. kodakarensis PCNA;
(c) a polynucleotide encoding the amino acid sequence of SEQ ID NO: 16 or a sequence having at least 90% sequence identity to the amino acid sequence of SEQ ID NO: 16;
(d) a Thermus thermophile HU-family DNA binding protein; or
(f) a polynucleotide encoding the amino acid sequence of SEQ ID NO: 16 or a sequence having 90% sequence identity to the amino acid sequence of SEQ ID NO: 16.

7. The engineered nucleic acid processing enzyme of claim 1, wherein the nucleic acid binding protein comprises:
(a) an amino acid sequence having at least 90%, at least 91%, at least 92%, at least 93%, at least 94%, at least 95%, at least 96%, at least 97%, at least 98%, or at least 99% sequence identity to the amino acid sequence of SEQ ID NO: 10; or
(b) an amino acid sequence having 90%, 91%, 92%, 93%, 94%, 95%, 96%, 97%, 98%, or 99% sequence identity to the amino acid sequence of SEQ ID NO: 10.

8. The engineered nucleic acid processing enzyme of claim 1, wherein the nucleic acid binding domain comprises an amino acid sequence set forth in SEQ ID NO: 4, 5, or 6 or an amino acid sequence having at least 90% sequence identity to the amino acid sequence set forth in SEQ ID NO: 4, 5, or 6.

9. The engineered nucleic acid processing enzyme of claim 1, wherein the engineered nucleic acid processing enzyme further comprises a tag protein selected from the group consisting of an affinity tag, a fluorescent tag, or an expression and/or solubility enhancement tag.

10. The engineered nucleic acid processing enzyme of claim 8, wherein the engineered nucleic acid processing enzyme comprises:
(a) an hexahistidine tag (his-tag);
(b) an amino acid sequence of SEQ ID NO: 9; or an amino acid sequence having at least 90% sequence identity to the amino acid sequence of SEQ ID NO: 9;
(c) a short peptide C-terminal tag;
(d) an amino acid sequence of SEQ ID NO: 10;
(e) an amino acid sequence having at least 90% sequence identity to the amino acid sequence of SEQ ID NO: 10; or
(f) an endoprotein cleavage sequence comprising the amino acid sequence of SEQ ID NO: 11, SEQ ID NO: 12, SEQ ID NO: 13, SEQ ID NO: 14, or SEQ ID NO: 15;
(g) an amino acid sequence of SEQ ID NO: 9; or an amino acid sequence having 90% sequence identity to the amino acid sequence of SEQ ID NO: 9;
(h) an amino acid sequence having 90% sequence identity to the amino acid sequence of SEQ ID NO: 10.

11. The engineered nucleic acid processing enzyme of claim 1, wherein the engineered nucleic acid processing enzyme:
(a) is thermophilic; and/or
(b) is resistant to thermal inactivation when compared to a wild-type polymerase; or
(c) is resistant to thermal inactivation at a temperature from about 53° C. to about 75° C.; from about 55° C. to about 75° C.; from about 60° C. to about 75° C.; from about 53° C. to about 68° C.; from about 55° C. to about 68° C.; from about 45° C. to about 68° C.; or from about 50° C. to about 68° C.; or
(d) is resistant to thermal inactivation at a temperature of about 68° C.

12. The engineered nucleic acid processing enzyme of claim 1, wherein the engineered nucleic acid processing enzyme possesses enhanced half-life when compared to a wild-type polymerase at a temperature from about 53° C. to about 75° C.; from about 55° C. to about 75° C.; from about 60° C. to about 75° C.; from about 53° C. to about 68° C.; from about 55° C. to about 68° C.; from about 45° C. to about 68° C.; or from about 50° C. to about 68° C.

13. An isolated nucleic acid molecule encoding the engineered thermostable reverse transcriptase of claim 1.

14. An expression vector comprising the isolated nucleic acid molecule of claim 12.

15. A host cell transfected with the expression vector of claim 13.

16. A method of using the engineered thermostable reverse transcriptase of claim 1, the method comprising contacting the engineered thermostable reverse transcriptase with a nucleic acid template under suitable conditions to produce a polymerized nucleic acid product, wherein the nucleic acid template is an RNA, a DNA, or a nucleic acid comprising an unnatural nucleotide.

17. A nucleic acid extension method comprising:
(a) contacting a target nucleic acid molecule with the engineered nucleic acid processing enzyme of claim 1 and a plurality of nucleic acid barcoded molecules comprising a barcode sequence, and
(b) incubating the target nucleic acid, the engineered nucleic acid processing enzyme and barcoded molecules under conditions in which the barcoded molecules are extended by the engineered thermostable reverse transcriptase;
wherein:
(i) one of the plurality of nucleic acid barcoded molecules hybridizes to the target nucleic acid molecule;
(ii) the nucleic acid binding domain binds and stabilizes the target nucleic acid molecule-barcoded molecule complex; and
(iii) the polymerase domain extends the one of the plurality of nucleic acid barcoded molecules that is hybridized to the target nucleic acid molecule.

18. The nucleic acid extension method of claim 17, wherein the polymerase domain comprises an amino acid sequence having:
(a) at least 90%, at least 91%, at least 92%, at least 93%, at least 94%, at least 95%, at least 96%, at least 97%, at least 98%, or at least 99% sequence identity to the amino acid sequence of SEQ ID NO: 1;
(b) at least 95% identity to the amino acid sequence of SEQ ID NO: 1;
(c) at least 97% identity to the amino acid sequence of SEQ ID NO: 1;
(d) at least about 10, at least about 15, or at least about 20 substitutions in the amino acid sequence of SEQ ID NO: 1; or
(e) at least 97% identity to the amino acid sequence of SEQ ID NO: 1 and at least about 15 substitutions in the amino acid sequence of SEQ ID NO: 1; or
(f) 90%, 91%, 92%, 93%, 94%, 95%, 96%, 97%, 98%, or 99% sequence identity to the amino acid sequence of SEQ ID NO: 1.

19. The nucleic acid extension method of claim 17, wherein:
(a) the polymerase domain comprises a substitution at positions 141 and 143 of SEQ ID NO: 1 or 2;
(b) the polymerase domain comprises a substitution at position 141 of SEQ ID NOs: 2;
(c) the engineered nucleic acid processing enzyme lacks proofreading activity; or
(d) the engineered nucleic acid processing enzyme comprises the amino acid sequence of SEQ ID NO: 1, 2 or 3; or
(e) the nucleic acid binding protein comprises an amino acid sequence having at least 90%, at least 91%, at least 92%, at least 93%, at least 94%, at least 95%, at least 96%, at least 97%, at least 98%, or at least 99% sequence identity to the amino acid sequence of SEQ ID NO: 4, 5, 6, or 10;
(f) the nucleic acid binding domain comprises an amino acid sequence set forth in SEQ ID NO: 4, 5, or 6; or
(g) the nucleic acid binding protein comprises an amino acid sequence having 90%, 91%, 92%, 93%, 94%, 95%, 96%, 97%, 98%, or 99% sequence identity to the amino acid sequence of SEQ ID NO: 4, 5, 6, or 10.

20. The nucleic acid extension method of claim 17, wherein the target nucleic acid molecule is further contacted with a sliding clamp molecule selected from an archea, an eucarya, or a bacteriophage sliding clamp protein.

21. The nucleic acid extension method of claim 20, wherein the sliding clamp protein is selected from E. coli polymerase β subunit; T4 bacteriophage gp45; T. gorgonarius PCNA; or T. kodakarensis PCNA.
